(19) United States
(12) Patent Application Publication
(10) Pub. No.: US 2012/0052097 A1
(43) Pub. Date: Mar. 1, 2012

(54) THERAPEUTIC PEPTIDE-POLYMER CONJUGATES, PARTICLES, COMPOSITIONS, AND RELATED METHODS

(75) Inventors: Oliver S. Fetzer, Needham, MA (US); Jungyeon Hwang, Lexington, MA (US); Patrick Lim Soo, Somerville, MA (US); Pei-Sze Ng, Cambridge, MA (US); Sonke Svenson, Arlington, MA (US)

(73) Assignee: Cerulean Pharma Inc., Cambridge, MA (US)

(21) Appl. No.: 13/212,971

(22) Filed: Aug. 18, 2011

Related U.S. Application Data
(60) Provisional application No. 61/375,771, filed on Aug. 20, 2010.
(60) Provisional application No. 61/477,827, filed on Apr. 21, 2011.

(51) Int. Cl. A61K 47/48; A61K 9/14; A61P 35/00; B32B 5/16; A61P 37/00; A61P 9/00; A61P 31/00; C08G 63/91; A61P 29/00

(52) U.S. Cl. 424/400; 525/54.1; 424/78.17; 428/402

(57) ABSTRACT

Described herein are conjugates (e.g., therapeutic peptide-polymer conjugates and protein-polymer conjugates) and particles, which can be used, for example, in the treatment of a disorder such as cancer. Also described herein are mixtures, compositions and dosage forms containing the particles, methods of using the particles (e.g., to treat a disorder), kits including the conjugates (e.g., therapeutic peptide-polymer conjugates and protein-polymer conjugates) and particles, methods of making the conjugates (e.g., therapeutic peptide-polymer conjugates and protein-polymer conjugates) and particles, methods of storing the particles and methods of analyzing the particles.

### CLAIM OF PRIORITY

This application claims priority to U.S. Ser. No. 61/375,771, filed Aug. 20, 2010 and U.S. Ser. No. 61/477,827, filed Apr. 21, 2011, the contents of both of which are incorporated herein by reference.

### BACKGROUND OF INVENTION

The delivery of a therapeutic peptide with controlled release of the therapeutic peptide is desirable to provide optimal use and effectiveness. Controlled release polymer systems may increase the efficacy of the therapeutic peptide and minimize problems with patient compliance.

### SUMMARY OF INVENTION

Described herein are particles, which can be used, for example, in the delivery of a therapeutic peptide or protein, for example, in the treatment of cancer, inflammatory disorders (e.g., an inflammatory disorder that includes an inflammatory disorder caused by, e.g., an infectious disease) or autoimmune disorders, cardiovascular diseases, or other disorders (e.g., infectious diseases). The particles, in general, include a hydrophilic-hydrophobic polymer (e.g., a di-block or tri-block co-polymer) and a therapeutic peptide or protein. In some embodiments, the particle also includes a hydrophobic polymer or a surfactant. In general, the therapeutic peptide is attached to a polymer, for example a hydrophilic-hydrophobic polymer, or if present, a hydrophobic polymer. In embodiments where the therapeutic peptide or protein is charged, the particle can also include a counterion to the therapeutic peptide. Also described herein are conjugates such as therapeutic peptide or protein-polymer conjugates, mixtures, compositions and dosage forms containing the particles or conjugates, methods of using the particles (e.g., to treat a disorder), kits including the therapeutic peptide or protein-polymer conjugates and particles, methods of making the therapeutic peptide or protein-polymer conjugates and particles, methods of storing the particles and methods of analyzing the particles.

In one aspect, the disclosure features a particle comprising:

a) a plurality of hydrophobic polymers;

b) a plurality of hydrophilic-hydrophobic polymers; and

c) a plurality of therapeutic peptides or proteins, wherein at least a portion of the plurality of therapeutic peptides or proteins are covalently attached to either of a hydrophobic polymer of a) or the hydrophilic-hydrophobic polymer b).

In some embodiments, the particle also includes a hydrophobic moiety such as chitosan, poly(vinyl alcohol), or a poloxamer.

In some embodiments, at least a portion of the hydrophobic polymers of a) are not covalently attached to a therapeutic peptide or protein of c). In some embodiments, at least a portion of the hydrophobic polymers of a) are covalently attached to a therapeutic peptide or protein of c), e.g., at least a portion of the hydrophobic polymers of a) are covalently attached to a single therapeutic peptide or protein of c) or at least a portion of the hydrophobic polymers of a) are covalently attached to a plurality of therapeutic peptides or proteins of c).

In some embodiments, at least a portion of the hydrophobic polymers of a) are directly covalently attached to a therapeutic peptide or protein of c) (e.g., at the carboxy terminal or hydroxyl terminal of the hydrophobic polymers). In some embodiments, at least a portion of the therapeutic peptides or proteins of c) are covalently attached to the hydrophobic polymer via a linker. Exemplary linkers include a linker that comprises a moiety formed using “click chemistry” (e.g., as described in WO 2006/115547), and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the therapeutic peptide or protein does not need to be cleaved to be active, e.g., the length of the linker is at least about 20 angstroms (e.g., at least about 24 angstroms).

In some embodiments, at least a portion of the hydrophobic polymers of a) are covalently attached to at least a portion of the therapeutic peptides or proteins of c) through the amino terminal of the therapeutic peptide or protein; at least a portion of the hydrophobic polymers of a) are covalently attached to at least a portion of the therapeutic peptides or proteins of c) through the carboxy terminal of the therapeutic peptide or proteins and/or at least a portion of the hydrophobic polymers of a) are covalently attached to at least a portion of the therapeutic peptides or proteins of c) through an amino acid side of the therapeutic peptide oe protein.

In some embodiments, at least a portion of the hydrophobic polymers of a) are coupled with a moiety that can dampen the pH of the hydrophobic polymer or particle. Exemplary pH dampening moieties include weakly basic salts such as calcium carbonate, magnesium hydroxide, and zinc carbonate, and proton sponges (e.g., including one or more amine groups) such as a polyamine.

In some embodiments, at least a portion of the hydrophilic-hydrophobic polymers of b) are covalently attached to a therapeutic peptide or protein of c). In some embodiments, at least a portion of the hydrophilic-hydrophobic polymers of b) are covalently attached to a single therapeutic peptide or protein of c). In some embodiments, at least a portion of the hydrophilic-hydrophobic polymers of b) are covalently attached to a plurality of therapeutic peptides or protein of c).

In some embodiments, at least a portion of the hydrophilic-hydrophobic polymers of b) are directly covalently attached to a therapeutic peptide or protein of c). In some embodiments, at least a portion of the therapeutic peptides or proteins of c) are covalently attached to a hydrophilic-hydrophobic polymer of b) via a linker. Exemplary linkers include a linker that comprises a moiety formed using “click chemistry” (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the therapeutic peptide or protein does not need to be cleaved to be active, e.g., the length of the linker is at least about 20 angstroms (e.g., at least about 24 angstroms).

In some embodiments, at least a portion of the hydrophilic-hydrophobic polymers of b) are covalently attached to a therapeutic peptide or protein of c) at the carboxy terminal or hydroxyl terminal of the hydrophobic polymers.

In some embodiments, at least a portion of the hydrophilic-hydrophobic polymers of b) are covalently attached to at least a portion of the therapeutic peptides or proteins of c) through the amino terminal of the therapeutic peptide or protein. In some embodiments, at least a portion of the hydrophilic-hydrophobic polymers of b) are covalently attached to at least a portion of the therapeutic peptides or proteins of c) through the carboxy terminal of the therapeutic peptide or protein. In some embodiments, at least a portion of the hydrophilic-hydrophobic polymers of b) are covalently attached to at least a portion of the therapeutic peptides or proteins of c) through an amino acid side of the therapeutic peptide or protein.

In some embodiments, the particle further comprises a plurality of additional therapeutic peptides or proteins, wherein the additional therapeutic peptides or proteins differ from the therapeutic peptides or proteins of c), e.g., at least a portion of the plurality of the additional therapeutic peptides or proteins are attached to at least a portion of either the hydrophobic polymers of a) and/or the hydrophilic-hydrophobic polymers of b).

In some embodiments, at least a portion of the hydrophobic polymers of a) are copolymers of lactic and glycolic acid (i.e., PLGA). For example, in some embodiments, a portion of the hydrophobic polymers of a) are PLGA having a ratio of from about 15:85 or 25:75 to about 75:25 or 85:15 of lactic acid to glycolic acid, e.g., a ratio of about 50:50 of lactic acid to glycolic acid.

In some embodiments, the hydrophobic polymers of a) have a weight average molecular weight of from about 6 to about 12 kDa, for example from about 8 to about 10 kDa. In other embodiments, the hydrophobic polymers of a) have a weight average molecular weight of from about 4 to about 8 kDa. In some embodiments, the hydrophobic polymers of a) have a weight average molecular weight of from about 10 to about 100 kDa.

In some embodiments, the hydrophobic polymers of a) comprise from about 35 to about 80% by weight of the particle.

In some embodiments, at least a portion of the hydrophobic polymers of a) are covalently attached to a therapeutic peptide or protein and a portion of the hydrophobic polymers of a) are attached to a plurality of therapeutic peptides or proteins.

In some embodiments, the hydrophilic-hydrophobic polymers of b) are block co-polymers. Exemplary block copolymers include a neutral hydrophilic block (e.g., which can enhance circulation), and a pH-responsive block (e.g., which can promote endosomal escape). Exemplary pH responsive blocks include those having a cis-acetonityl, hydrazone, or acetal linker, which can be hydrolyzed, for example, from pH 4 to 6.5. In some embodiment, the polymer includes a reversible peptide conjugation site, for example, which can provide means for peptide release from the carrier when reaching the cytosol (e.g., a thiol).

In some embodiments, the hydrophilic-hydrophobic polymers of b) are di-block co-polymers (e.g., PEG-PLGA). In some embodiments, the hydrophilic-hydrophobic polymers of b) are tri-block-co-polymer (e.g., PEG-PLGA-PEG). In some embodiments, the hydrophobic portion of at least a portion of the hydrophilic-hydrophobic polymers of b) has a hydroxyl terminal end. In some embodiments, the hydrophobic portion of at least a portion of the hydrophilic-hydrophobic polymers of b) have a hydroxyl terminal end and the hydroxyl terminal end is capped (e.g., capped with an acyl moiety). For example, in some embodiments, the hydrophobic portion of at least a portion of the hydrophilic-hydrophobic polymers of b) have a hydroxyl terminal end and the hydroxyl terminal end is capped with an acyl moiety.

In some embodiments, the hydrophobic portion of the hydrophilic-hydrophobic polymers of b) comprises copolymers of lactic and glycolic acid (i.e., PLGA). In some embodiments, the hydrophobic portion of the hydrophilic-hydrophobic polymers of b) comprises PLGA having a ratio of from about 15:85 or 25:75 to about 75:25 or 85:15 of lactic acid to glycolic acid, e.g., a ratio of about 50:50 of lactic acid to glycolic acid.

In some embodiments, the hydrophilic portion of the hydrophilic-hydrophobic polymers of b) has a weight average molecular weight of from about 1 to about 6 kDa (e.g., from about 2 to about 6 kDa). In some embodiments, the hydrophobic portion of the hydrophilic-hydrophobic polymers of b) has a weight average molecular weight of from about 8 to about 13 kDa.

In some embodiments, the plurality of hydrophilic-hydrophobic polymers of b) is from about 5 to about 25 weight % of said particle (e.g., from about 10 to about 25 weight %).

In some embodiments, the hydrophilic portion of the hydrophilic-hydrophobic polymers of b) comprises PEG.

In some embodiments, the hydrophilic portion of said hydrophilic-hydrophobic polymer terminates in a methoxy.

In some embodiments, at least a portion of the hydrophilic-hydrophobic polymers of b) are covalently attached to a therapeutic peptide or protein and a portion of the hydrophilic-hydrophobic polymers of b) are attached to a plurality of therapeutic peptides or proteins.

In some embodiments, the therapeutic peptide is a therapeutic peptide described herein. In some embodiments, the therapeutic peptide comprises from about 2 to about 50 amino acid residues, e.g., about 2 to about 40 amino acid residues or about 2 to about 30 amino acid residues.

In some embodiments, the protein is a protein described herein.

In some embodiments, at least a portion of the therapeutic peptides or proteins are chemically modified.

In some embodiments, the plurality of therapeutic peptides are from about 1 to about 90 weight % of said particle (e.g., from about 50% to about 90%, from about 70% to about 90%, from about 10% to 50%, from about 10% to about 30%).

In some embodiments, the particle further comprises a surfactant. In some embodiments, the surfactant is a polymer, e.g., the surfactant is PVA. In some embodiments, the PVA has a weight average molecular weight of from about 23 to about 26 kDa. In some embodiments, the surfactant is from about 15 to about 35 weight % of said particle.

In some embodiments, the particle further comprises a counterion. For example, in embodiments where the therapeutic peptide is a charged peptide, the particle can include a counterion, wherein the counterion has a charge opposite to that of the charge on the therapeutic peptide. In some embodiments, the ratio of the charge of the therapeutic peptide to the charge of the counterion in the particle is from about 1:1.5 to about 1.5:1 (e.g., from about 1.25:1 to about 1:1.25, or about 1:1).

In some embodiments, the counterion can act as a surfactant (e.g., a single moiety can function as both a counterion and also a surfactant).

In some embodiments, the diameter of the particle is less than about 200 nm (e.g., less than about 150 nm).

In some embodiments, the surface of the particle is substantially coated with a polymer such as PEG.

In some embodiments, the zeta potential of the particle is from about −10 to about 10 mV (e.g., from about −5 to about 5 mV).

In some embodiments, the particle is chemically stable under conditions, comprising a temperature of 23 degrees Celsius and 60% percent humidity for at least 1 day (e.g., at least 7 days, at least 14 days, at least 21 days, at least 30 days).

In some embodiments, the particle is a lyophilized particle.

In some embodiments, the particle is formulated into a pharmaceutical composition.

In some embodiments, the surface of the particle is substantially free of a targeting agent.

In some embodiments, the therapeutic peptide or protein is attached to a hydrophobic polymer of a) and the therapeutic peptide or protein-hydrophobic polymer conjugate has one or more of the following properties:

i) the hydrophobic polymer attached to the therapeutic peptide or protein can be a homopolymer or a polymer made up of more than one kind of monomeric subunit;

ii) the hydrophobic polymer attached to said therapeutic peptide or protein has a weight average molecular weight of about 4-15 kDa;

iii) the hydrophobic polymer is made up of a first and a second type of monomeric subunit, and the ratio of the first to second type of monomeric subunit in said hydrophobic polymer attached to the therapeutic peptide or protein is from about 15:85 or 25:75 to about 75:25 or 85:15, e.g., about 50:50;

iv) the hydrophobic polymer is PLGA; and

v) the therapeutic peptide or protein is about 1 to about 100 weight % of said particle (e.g., from about 50% to about 100%, from about 70% to about 100%, from about 50% to 90%).

In some embodiments, the hydrophobic polymer attached to the therapeutic peptide or protein has a weight average molecular weight of about 4-15 kDa, e.g., 6-12 kDa, e.g., 8-10 kDa.

In some embodiments, the hydrophilic-hydrophobic polymers of b) have one or more of the following properties:

i) the hydrophilic portion has a weight average molecular weight of about 1-6 kDa (e.g., 2-6 kDa),

ii) the hydrophobic polymer has a weight average molecular weight of about 4-15 kDa; iii) the hydrophilic polymer is PEG;

iv) the hydrophobic polymer is made up of a first and a second type of monomeric subunit, and the ratio of the first to second type of monomeric subunit in the hydrophobic polymer is from about 15:85 or 25:75 to about 75:25 or 85:15, e.g., about 50:50; and

v) the hydrophobic polymer is PLGA.

In some embodiments, if the weight average molecular weight of the hydrophilic portion of the hydrophilic-hydrophobic polymer of b) is about 1-3 kDa, e.g., about 2 kDa, the ratio of the weight average molecular weight of the hydrophilic portion to the weight average molecular weight of the hydrophobic portion is between 1:3-1:7, and if the weight average molecular weight of the hydrophilic portion is about 4-6 kDa, e.g., about 5 kDa, the ratio of the weight average molecular weight of the hydrophilic portion to the weight average molecular weight of the hydrophobic portion is between 1:1-1:4.

In some embodiments, the hydrophilic portion of the hydrophilic-hydrophobic polymer of b) has a weight average molecular weight of about 2-6 kDa and the hydrophobic portion has a weight average molecular weight of between about 8-13 kDa.

In some embodiments, the hydrophilic portion of said hydrophilic-hydrophobic polymer of b) terminates in a methoxy.

In some embodiments, the therapeutic peptide is attached to a hydrophobic polymer of a) and the therapeutic peptide-hydrophobic polymer conjugate has one or more of the following properties:

i) the hydrophobic polymer attached to the therapeutic peptide can be a homopolymer or a polymer made up of more than one kind of monomeric subunit;

ii) the hydrophobic polymer attached to the therapeutic peptide has a weight average molecular weight of about 4-15 kDa;

iii) the hydrophobic polymer is made up of a first and a second type of monomeric subunit, and the ratio of the first to second type of monomeric subunit in the hydrophobic polymer attached to the therapeutic peptide or protein is from about 15:85 or 25:75 to about 75:25 or 85:15, e.g., about 50:50; and

iv) the hydrophobic polymer is PLGA.

In some embodiments, the particle further comprises a surfactant (e.g. PVA).

In another aspect, the disclosure features a particle comprising:

a) a plurality of therapeutic peptide or protein-polymer conjugates, comprising a therapeutic peptide or protein attached to a hydrophobic polymer; and

b) a plurality of hydrophilic-hydrophobic polymers.

In some embodiments, the particle further comprises a hydrophobic polymer (e.g., PLGA).

In some embodiments, the particle also includes a hydrophobic moiety such as chitosan, poly(vinyl alcohol), or a poloxamer.

In some embodiments, the therapeutic peptide or protein is covalently attached to the hydrophobic polymer via a linker. Exemplary linkers include a linker that comprises a moiety formed using “click chemistry” (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the therapeutic peptide or protein does not need to be cleaved to be active, e.g., the length of the linker is at least about 20 angstroms (e.g., at least about 24 angstroms).

In some embodiments, the particle further comprises a plurality of additional therapeutic peptides or proteins, wherein the additional therapeutic peptides or proteins differ from the therapeutic peptides or proteins of a). In some embodiments, at least a portion of the plurality of the additional therapeutic peptides or proteins are attached to hydrophobic polymers and/or at least a portion of the hydrophilic-hydrophobic polymers of b).

In some embodiments, at least a portion of the hydrophobic polymers of a) are copolymers of lactic and glycolic acid (i.e., PLGA). For example, in some embodiments, a portion of the hydrophobic polymers of a) are PLGA having a ratio of from about 15:85 or 25:75 to about 75:25 or 85:15 of lactic acid to glycolic acid, e.g., a ratio of about 50:50 of lactic acid to glycolic acid.

In some embodiments, the hydrophobic polymers of a) have a weight average molecular weight of from about 6 to about 12 kDa, for example from about 8 to about 10 kDa. In other embodiments, the hydrophobic polymers of a) have a weight average molecular weight of from about 4 to about 8 kDa. In some embodiments, the hydrophobic polymers of a) have a weight average molecular weight of from about 10 to about 100 kDa.

In some embodiments, the hydrophobic polymers of a) comprise from about 35 to about 80% by weight of the particle.

In some embodiments, the hydrophilic-hydrophobic polymers of b) are block co-polymers, e.g., the hydrophilic-hydrophobic polymers of b) are di-block co-polymers. In some embodiments, the hydrophilic-hydrophobic polymers of b) are block co-polymers. Exemplary block copolymers include a neutral hydrophilic block (e.g., which can enhance circulation), and a pH-responsive block (e.g., which can promote endosomal escape). Exemplary pH responsive blocks include those having a cis-acetonityl, hydrazone, or acetal linker, which can be hydrolyzed, for example, from pH 4 to 6.5. In some embodiment, the polymer includes a reversible peptide conjugation site, for example, which can provide means for peptide release from the carrier when reaching the cytosol (e.g., a thiol).

In some embodiments, the hydrophobic portion of at least a portion of the hydrophilic-hydrophobic polymers of b) has a hydroxyl terminal end. In some embodiments, the hydrophobic portion of at least a portion of the hydrophilic-hydrophobic polymers of b) have a hydroxyl terminal end and the hydroxyl terminal end is capped (e.g., capped with an acyl moiety). For example, in some embodiments, the hydrophobic portion of at least a portion of the hydrophilic-hydrophobic polymers of b) have a hydroxyl terminal end and the hydroxyl terminal end is capped with an acyl moiety.

In some embodiments, at least a portion of the hydrophobic polymers of a) are coupled with a moiety that can dampen the pH of the hydrophobic polymer or particle. Exemplary pH dampening moieties include weakly basic salts such as calcium carbonate, magnesium hydroxide, and zinc carbonate, and proton sponges (e.g., including one or more amine groups) such as a polyamine.

In some embodiments, the hydrophobic portion of the hydrophilic-hydrophobic polymers of b) comprises copolymers of lactic and glycolic acid (i.e., PLGA). In some embodiments, the hydrophobic portion of the hydrophilic-hydrophobic polymers of b) comprises PLGA having a ratio of from about 15:85 or 25:75 to about 75:25 or 85:15 of lactic acid to glycolic acid, e.g., a ratio of about 50:50 of lactic acid to glycolic acid.

In some embodiments, the hydrophilic portion of the hydrophilic-hydrophobic polymers of b) has a weight average molecular weight of from about 1 to about 6 kDa (e.g., from about 2 to about 6 kDa). In some embodiments, the hydrophobic portion of the hydrophilic-hydrophobic polymers of b) has a weight average molecular weight of from about 8 to about 13 kDa.

In some embodiments, the plurality of hydrophilic-hydrophobic polymers of b) is from about 5 to about 25 weight % of said particle (e.g., from about 10 to about 25 weight %).

In some embodiments, the hydrophilic portion of the hydrophilic-hydrophobic polymers of b) comprises PEG.

In some embodiments, the hydrophilic portion of said hydrophilic-hydrophobic polymer terminates in a methoxy.

In some embodiments, the therapeutic peptide is a therapeutic peptide described herein. In some embodiments, the therapeutic peptide comprises from about 2 to about 50 amino acid residues, e.g., about 2 to about 40 amino acid residues or about 2 to about 30 amino acid residues.

In some embodiments, the protein is a protein described herein.

In some embodiments, at least a portion of the therapeutic peptide are chemically modified.

In some embodiments, the plurality of therapeutic peptides are from about 1 to about 50 weight % of said particle (e.g., from about 1% to about 20%).

In some embodiments, the particle further comprises a surfactant. In some embodiments, the surfactant is a polymer, e.g., the surfactant is PVA. In some embodiments, the PVA has a weight average molecular weight of from about 23 to about 26 kDa. In some embodiments, the surfactant is from about 15 to about 35 weight % of said particle.

In some embodiments, the particle further comprises a counterion. For example, in embodiments where the therapeutic peptide is a charged peptide, the particle can include a counterion, wherein the counterion has a charge opposite to that of the charge on the therapeutic peptide or protein. In some embodiments, the ratio of the charge of the therapeutic peptide or protein to the charge of the counterion in the particle is from about 1:1.5 to about 1.5:1 (e.g., from about 1.25:1 to about 1:1.25, or about 1:1).

In some embodiments, the counterion can act as a surfactant (e.g., a single moiety can function as both a counterion and also a surfactant).

In some embodiments, the diameter of the particle is less than about 200 nm (e.g., less than about 150 nm).

In some embodiments, the surface of the particle is substantially coated with a polymer such as PEG.

In some embodiments, the zeta potential of the particle is from about −10 to about 10 mV (e.g., from about −5 to about 5 mV).

In some embodiments, the particle is chemically stable under conditions, comprising a temperature of 23 degrees Celsius and 60% percent humidity for at least 1 day (e.g., at least 7 days, at least 14 days, at least 21 days, at least 30 days).

In some embodiments, the particle is a lyophilized particle.

In some embodiments, the particle is formulated into a pharmaceutical composition.

In some embodiments, the surface of the particle is substantially free of a targeting agent.

In some embodiments, the therapeutic peptide or protein is attached to a hydrophobic polymer of a) and the therapeutic peptide or protein-hydrophobic polymer conjugate has one or more of the following properties:

i) the hydrophobic polymer attached to said therapeutic peptide or protein can be a homopolymer or a polymer made up of more than one kind of monomeric subunit;

ii) the hydrophobic polymer attached to said therapeutic peptide or protein has a weight average molecular weight of about 4-15 kDa;

iii) the hydrophobic polymer is made up of a first and a second type of monomeric subunit, and the ratio of the first to second type of monomeric subunit in said hydrophobic polymer attached to the therapeutic peptide or protein is from about 15:85 or 25:75 to about 75:25 or 85:15, e.g., about 50:50;

iv) the hydrophobic polymer is PLGA; and

v) the therapeutic peptide is about 1 to about 20 weight % of the particle.

In some embodiments, the hydrophobic polymer attached to the therapeutic peptide or protein has a weight average molecular weight of about 4-15 kDa, e.g., 6-12 kDa, e.g., 8-10 kDa.

In some embodiments, the hydrophilic-hydrophobic polymers of b) have one or more of the following properties:

i) the hydrophilic portion has a weight average molecular weight of about 1-6 kDa (e.g., 2-6 kDa),

ii) the hydrophobic polymer has a weight average molecular weight of about 4-15 kDa;

iii) the hydrophilic polymer is PEG;

iv) the hydrophobic polymer is made up of a first and a second type of monomeric subunit, and the ratio of the first to second type of monomeric subunit in the hydrophobic polymer is from about 15:85 or 25:75 to about 75:25 or 85:15, e.g., about 50:50; and

v) the hydrophobic polymer is PLGA.

In some embodiments, if the weight average molecular weight of the hydrophilic portion of the hydrophilic-hydrophobic polymer of b) is about 1-3 kDa, e.g., about 2 kDa, the ratio of the weight average molecular weight of the hydrophilic portion to the weight average molecular weight of the hydrophobic portion is between 1:3-1:7, and if the weight average molecular weight of the hydrophilic portion is about 4-6 kDa, e.g., about 5 kDa, the ratio of the weight average molecular weight of the hydrophilic portion to the weight average molecular weight of the hydrophobic portion is between 1:1-1:4.

In some embodiments, the hydrophilic portion of the hydrophilic-hydrophobic polymer of b) has a weight average molecular weight of about 2-6 kDa and the hydrophobic portion has a weight average molecular weight of between about 8-13 kDa.

In some embodiments, the hydrophilic portion of said hydrophilic-hydrophobic polymer of b) terminates in a methoxy.

In some embodiments, the therapeutic peptide is attached to a hydrophobic polymer of a) and the therapeutic peptide-hydrophobic polymer conjugate has one or more of the following properties:

i) the hydrophobic polymer attached to the therapeutic peptide or protein can be a homopolymer or a polymer made up of more than one kind of monomeric subunit;

ii) the hydrophobic polymer attached to the therapeutic peptide or protein has a weight average molecular weight of about 4-15 kDa;

iii) the hydrophobic polymer is made up of a first and a second type of monomeric subunit, and the ratio of the first to second type of monomeric subunit in the hydrophobic polymer attached to the therapeutic peptide or protein is from about 15:85 or 25:75 to about 75:25 or 85:15, e.g., about 50:50; and

iv) the hydrophobic polymer is PLGA.

In some embodiments, the particle further comprises a surfactant (e.g. PVA).

In some embodiments, the therapeutic peptide is a therapeutic peptide described herein. In some embodiments, the therapeutic peptide comprises from about 2 to about 50 amino acid residues, e.g., about 2 to about 40 amino acid residues or about 2 to about 30 amino acid residues.

In some embodiments, the protein is a protein described herein.

In some embodiments, at least a portion of the therapeutic peptide or protein are chemically modified.

In some embodiments, the plurality of therapeutic peptides or proteins are from about 1 to about 100 weight % of said particle (e.g., from about 50% to about 100%, from about 70% to about 100%, from about 50% to about 90%).

In some aspects, the disclosure features a particle comprising:

a) optionally a plurality of hydrophobic polymers; and

b) a plurality of therapeutic peptide or protein-hydrophilic-hydrophobic polymer conjugate, comprising a therapeutic peptide or protein attached to the hydrophilic-hydrophobic polymer.

In some embodiments, the particle is substantially free of hydrophobic polymers. In some embodiments, the particle also includes a hydrophobic moiety such as chitosan, poly(vinyl alcohol), or a poloxamer.

In some embodiments, the particle further comprises a plurality of hydrophilic-hydrophobic polymers, wherein each of said hydrophilic-hydrophobic polymers of said plurality comprises a hydrophilic portion attached to a hydrophobic portion.

In some embodiments, the hydrophobic-hydrophilic polymer of the conjugate of b) is covalently attached to the therapeutic peptide or protein via a linker. Exemplary linkers include a linker comprises a moiety formed using “click chemistry” (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the therapeutic peptide or protein does not need to be cleaved to be active, e.g., the length of the linker is at least about 20 angstroms (e.g., at least about 24 angstroms).

In some embodiments, the particle further comprises a plurality of additional therapeutic peptides or proteins, wherein the additional therapeutic peptides or proteins differ from the therapeutic peptides or proteins of b). In some embodiments, at least a portion of the plurality of the additional therapeutic peptides or proteins are attached to at least a portion of either the hydrophobic polymers of a) and/or hydrophilic-hydrophobic polymers. In some embodiments, at least a portion of the plurality of the additional therapeutic peptides or proteins are attached to at least a portion of the hydrophobic polymers of a).

In some embodiments, the particle comprises hydrophobic polymers. In some embodiments, at least a portion of the hydrophobic polymers of a) have a carboxy terminal end. In some embodiments, at least a portion of the hydrophobic polymers of a) have a hydroxyl terminal end. In some embodiments, at least a portion of the hydrophobic polymers of a) having a hydroxyl terminal end have the hydroxyl terminal end capped (e.g., capped with an acyl moiety).

In some embodiments, the terminal end of the hydrophobic polymer is modified (e.g., by reacting with a functional moiety), e.g., a hydroxy terminal end of the hydrophobic polymer is modified (e.g., by reacting with a functional moiety) and/or a carboxy terminal end of the hydrophobic polymer is modified (e.g., by reacting with a functional moiety). For example, a hydroxy terminal end or a carboxy terminal end is modified with a reactive moiety which can be used to attach a therapeutic peptide or protein to the polymer, e.g., through a linker. In some embodiments, the reactive moiety has not reacted with the therapeutic peptide or protein and remains on the polymer or is hydrolyzed in a subsequent reaction.

In some embodiments, at least a portion of the hydrophobic polymers of a) have both a carboxy terminal end and a hydroxyl terminal end and, e.g., at least a portion of the hydrophobic polymers of a) having a hydroxyl terminal end have the hydroxyl terminal end capped (e.g., capped with an acyl moiety).

In some embodiments, at least a portion of the hydrophobic polymers of a) are copolymers of lactic and glycolic acid (i.e., PLGA). For example, in some embodiments, a portion of the hydrophobic polymers of a) are PLGA having a ratio of from about 15:85 or 25:75 to about 75:25 or 85:15 of lactic acid to glycolic acid, e.g., a ratio of about 50:50 of lactic acid to glycolic acid.

In some embodiments, the hydrophobic polymers of a) have a weight average molecular weight of from about 6 to about 12 kDa, for example from about 8 to about 10 kDa. In other embodiments, the hydrophobic polymers of a) have a weight average molecular weight of from about 4 to about 8 kDa. In some embodiments, the hydrophobic polymers of a) have a weight average molecular weight of from about 10 to about 100 kDa.

In some embodiments, the hydrophobic polymers of a) comprise from about 35 to about 80% by weight of the particle.

In some embodiments, at least a portion of the hydrophobic polymers of a) are covalently attached to a therapeutic peptide or protein and a portion of the hydrophobic polymers of a) are attached to a plurality of therapeutic peptides or proteins.

In some embodiments, at least a portion of the hydrophobic polymers of a) are coupled with a moiety that can dampen the pH of the hydrophobic polymer or particle. Exemplary pH dampening moieties include weakly basic salts such as calcium carbonate, magnesium hydroxide, and zinc carbonate, and proton sponges (e.g., including one or more amine groups) such as a polyamine.

In some embodiments, the hydrophilic-hydrophobic polymers of b) are block co-polymers. In some embodiments, the hydrophilic-hydrophobic polymers of b) are block co-polymers. Exemplary block copolymers include a neutral hydrophilic block (e.g., which can enhance circulation), and a pH-responsive block (e.g., which can promote endosomal escape). Exemplary pH responsive blocks include those having a cis-acetonityl, hydrazone, or acetal linker, which can be hydrolyzed, for example, from pH 4 to 6.5. In some embodiment, the polymer includes a reversible peptide conjugation site, for example, which can provide means for peptide release from the carrier when reaching the cytosol (e.g., a thiol). In some embodiments, the hydrophilic-hydrophobic polymers of b) are di-block co-polymers (e.g., PEG-PLGA). In some embodiments, the hydrophilic-hydrophobic polymers of b) are tri-block-co-polymer (e.g., PEG-PLGA-PEG).

In some embodiments, the hydrophobic portion of at least a portion of the hydrophilic-hydrophobic polymers of b) has a hydroxyl terminal end. In some embodiments, the hydrophobic portion of at least a portion of the hydrophilic-hydrophobic polymers of b) have a hydroxyl terminal end and the hydroxyl terminal end is capped (e.g., capped with an acyl moiety). For example, in some embodiments, the hydrophobic portion of at least a portion of the hydrophilic-hydrophobic polymers of b) have a hydroxyl terminal end and the hydroxyl terminal end is capped with an acyl moiety.

In some embodiments, the hydrophobic portion of the hydrophilic-hydrophobic polymers of b) comprises copolymers of lactic and glycolic acid (i.e., PLGA). In some embodiments, the hydrophobic portion of the hydrophilic-hydrophobic polymers of b) comprises PLGA having a ratio of from about 15:85 or 25:75 to about 75:25 or 85:15 of lactic acid to glycolic acid, e.g., a ratio of about 50:50 of lactic acid to glycolic acid.

In some embodiments, the hydrophilic portion of the hydrophilic-hydrophobic polymers of b) has a weight average molecular weight of from about 1 to about 6 kDa (e.g., from about 2 to about 6 kDa). In some embodiments, the hydrophobic portion of the hydrophilic-hydrophobic polymers of b) has a weight average molecular weight of from about 8 to about 13 kDa.

In some embodiments, the plurality of hydrophilic-hydrophobic polymers of b) is from about 5 to about 25 weight % of said particle (e.g., from about 10 to about 25 weight %).

In some embodiments, the hydrophilic portion of the hydrophilic-hydrophobic polymers of b) comprises PEG.

In some embodiments, the hydrophilic portion of said hydrophilic-hydrophobic polymer terminates in a methoxy.

In some embodiments, at least a portion of the hydrophilic-hydrophobic polymers of b) are covalently attached to a therapeutic peptide or protein and a portion of the hydrophilic-hydrophobic polymers of b) are attached to a plurality of therapeutic peptides or proteins.

In some embodiments, the hydrophobic polymer has one or more of the following properties:

i) the hydrophobic polymer can be a homopolymer or a polymer made up of more than one kind of monomeric subunit;

ii) the hydrophobic polymer has a weight average molecular weight of about 4-15 kDa;

iii) the hydrophobic polymer is made up of a first and a second type of monomeric subunit, and the ratio of the first to second type of monomeric subunit in said hydrophobic polymer attached to said agent is from about 15:85 or 25:75 to about 75:25 or 85:15, e.g., about 50:50; and

iv) the hydrophobic polymer is PLGA.

In some embodiments, the hydrophobic polymer has a weight average molecular weight of about 4-15 kDa, e.g., 6-12 kDa, e.g., 8-10 kDa.

In some embodiments, the hydrophilic-hydrophobic polymers of b) have one or more of the following properties:

i) the hydrophilic portion has a weight average molecular weight of about 1-6 kDa (e.g., 2-6 kDa),

ii) the hydrophobic polymer has a weight average molecular weight of about 4-15 kDa;

iii) the hydrophilic polymer is PEG;

iv) the hydrophobic portion of the hydrophilic-hydrophobic polymer is made up of a first and a second type of monomeric subunit, and the ratio of the first to second type of monomeric subunit in the hydrophobic portion is from about 15:85 or 25:75 to about 75:25 or 85:15, e.g., about 50:50; and

v) the hydrophobic portion of the hydrophilic-hydrophobic polymer is PLGA.

In some embodiments, if the weight average molecular weight of the hydrophilic portion of the hydrophilic-hydrophobic polymer is about 1-3 kDa, e.g., about 2 kDa, the ratio of the weight average molecular weight of the hydrophilic portion to the weight average molecular weight of the hydrophobic portion is between 1:3-1:7, and if the weight average molecular weight of the hydrophilic portion is about 4-6 kDa, e.g., about 5 kDa, the ratio of the weight average molecular weight of the hydrophilic portion to the weight average molecular weight of the hydrophobic portion is between 1:1-1:4.

In some embodiments, the hydrophilic portion of the hydrophilic-hydrophobic polymer has a weight average molecular weight of about 2-6 kDa and the hydrophobic portion has a weight average molecular weight of between about 8-13 kDa.

In some embodiments, hydrophilic portion of said hydrophilic-hydrophobic polymer terminates in a methoxy.

In some embodiments, the hydrophobic polymer has one or more of the following properties:

i) the hydrophobic polymer can be a homopolymer or a polymer made up of more than one kind of monomeric subunit;

ii) the hydrophobic polymer has a weight average molecular weight of about 4-15 kDa;

iii) the hydrophobic polymer is made up of a first and a second type of monomeric subunit, and the ratio of the first to second type of monomeric subunit in the hydrophobic polymer is from about 15:85 or 25:75 to about 75:25 or 85:15, e.g., about 50:50; and

iv) the hydrophobic polymer is PLGA.

In some embodiments, the therapeutic peptide is a therapeutic peptide described herein. In some embodiments, the therapeutic peptide comprises from about 2 to about 50 amino acid residues, e.g., about 2 to about 40 amino acid residues or about 2 to about 30 amino acid residues.

In some embodiments, the protein is a protein described herein.

In some embodiments, at least a portion of the therapeutic peptide or protein is chemically modified.

In some embodiments, the plurality of therapeutic peptides or proteins are from about 1 to about 100 weight % of said particle (e.g., from about 50% to about 100%, from about 70% to about 100%, from about 50% to about 90%).

In some embodiments, the particle further comprises a surfactant. In some embodiments, the surfactant is a polymer, e.g., the surfactant is PVA. In some embodiments, the PVA has a weight average molecular weight of from about 23 to about 26 kDa. In some embodiments, the surfactant is from about 15 to about 35 weight % of said particle.

In some embodiments, the particle further comprises a counterion. For example, in embodiments where the therapeutic peptide is a charged peptide, the particle can include a counterion, wherein the counterion has a charge opposite to that of the charge on the therapeutic peptide. In some embodiments, the ratio of the charge of the therapeutic peptide or protein to the charge of the counterion in the particle is from about 1:1.5 to about 1.5:1 (e.g., from about 1.25:1 to about 1:1.25, or about 1:1).

In some embodiments, the counterion can act as a surfactant (e.g., a single moiety can function as both a counterion and also a surfactant).

In some embodiments, the diameter of the particle is less than about 200 nm (e.g., less than about 150 nm).

In some embodiments, the surface of the particle is substantially coated with a polymer such as PEG.

In some embodiments, the zeta potential of the particle is from about −10 to about 10 mV (e.g., from about −5 to about 5 mV).

In some embodiments, the particle is chemically stable under conditions, comprising a temperature of 23 degrees Celsius and 60% percent humidity for at least 1 day (e.g., at least 7 days, at least 14 days, at least 21 days, at least 30 days).

In some embodiments, the particle is a lyophilized particle.

In some embodiments, the particle is formulated into a pharmaceutical composition.

In some embodiments, the surface of the particle is substantially free of a targeting agent.

In some aspects, the disclosure features a particle comprising:

a) optionally, a plurality of hydrophobic polymers;

b) a plurality of hydrophilic-hydrophobic polymer-conjugates, wherein the hydrophilic-hydrophobic polymer conjugate comprises a hydrophilic-hydrophobic polymer attached to a charged peptide; and

c) a plurality of charged therapeutic peptides or proteins, wherein the charge of the therapeutic peptide or protein is opposite the charge of the peptide conjugated to the hydrophilic-hydrophobic polymer, and wherein the charged therapeutic peptide or protein forms a non-covalent bond (e.g., an ionic bond) with the charged peptide or protein of the hydrophilic-hydrophobic polymer-conjugate.

In some embodiments, the particle is substantially free of hydrophobic polymers. In some embodiments, the particle also includes a hydrophobic moiety such as chitosan, poly(vinyl alcohol), or a poloxamer.

In some embodiments, the particle further comprises a hydrophilic-hydrophobic polymer such as block co-polymer (e.g., PEG-PLGA). Exemplary block copolymers include a neutral hydrophilic block (e.g., which can enhance circulation), and a pH-responsive block (e.g., which can promote endosomal escape). Exemplary pH responsive blocks include those having a cis-acetonityl, hydrazone, or acetal linker, which can be hydrolyzed, for example, from pH 4 to 6.5. In some embodiment, the polymer includes a reversible peptide conjugation site, for example, which can provide means for peptide release from the carrier when reaching the cytosol (e.g., a thiol). In some embodiments, the hydrophilic-hydrophobic polymers of b) are di-block co-polymers (e.g., PEG-PLGA). In some embodiments, the hydrophilic-hydrophobic polymers of b) are tri-block-co-polymer (e.g., PEG-PLGA-PEG).

In some embodiments, the block co-polymer is a di-block or tri-block co-polymer. Exemplary block copolymers include a neutral hydrophilic block (e.g., which can enhance circulation), and a pH-responsive block (e.g., which can promote endosomal escape). Exemplary pH responsive blocks include those having a cis-acetonityl, hydrazone, or acetal linker, which can be hydrolyzed, for example, from pH 4 to 6.5. In some embodiment, the polymer includes a reversible peptide conjugation site, for example, which can provide means for peptide release from the carrier when reaching the cytosol (e.g., a thiol).

In some embodiments, the hydrophobic-hydrophilic polymer of the conjugate of b) is covalently attached to the therapeutic peptide or protein via a linker. Exemplary linkers include a linker comprises a moiety formed using “click chemistry” (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the therapeutic peptide or protein does not need to be cleaved to be active, e.g., the length of the linker is at least about 20 angstroms (e.g., at least about 24 angstroms).

In some embodiments, the particle further comprises a plurality of additional therapeutic peptides or proteins, wherein the additional therapeutic peptides or proteins differ from the therapeutic peptides or proteins of b). In some embodiments, at least a portion of the plurality of the additional therapeutic peptides or proteins are attached to at least a portion of either the hydrophobic polymers of a) and/or hydrophilic-hydrophobic polymers. In some embodiments, at least a portion of the plurality of the additional therapeutic peptides or proteins are attached to at least a portion of the hydrophobic polymers of a).

In some embodiments, the particle comprises hydrophobic polymers. In some embodiments, at least a portion of the hydrophobic polymers of a) have a carboxy terminal end. In some embodiments, at least a portion of the hydrophobic polymers of a) have a hydroxyl terminal end. In some embodiments, at least a portion of the hydrophobic polymers of a) having a hydroxyl terminal end have the hydroxyl terminal end capped (e.g., capped with an acyl moiety).

In some embodiments, the terminal end of the hydrophobic polymer is modified (e.g., by reacting with a functional moiety), e.g., a hydroxy terminal end of the hydrophobic polymer is modified (e.g., by reacting with a functional moiety) and/or a carboxy terminal end of the hydrophobic polymer is modified (e.g., by reacting with a functional moiety). For example, a hydroxy terminal end or a carboxy terminal end is modified with a reactive moiety which can be used to attach a therapeutic peptide or protein to the polymer, e.g., through a linker. In some embodiments, the reactive moiety has not reacted with the therapeutic peptide or protein and remains on the polymer or is hydrolyzed in a subsequent reaction.

In some embodiments, at least a portion of the hydrophobic polymers of a) have both a carboxy terminal end and a hydroxyl terminal end and, e.g., at least a portion of the hydrophobic polymers of a) having a hydroxyl terminal end have the hydroxyl terminal end capped (e.g., capped with an acyl moiety).

In some embodiments, at least a portion of the hydrophobic polymers of a) is copolymers of lactic and glycolic acid (i.e., PLGA). For example, in some embodiments, a portion of the hydrophobic polymers of a) are PLGA having a ratio of from about 15:85 or 25:75 to about 75:25 or 85:15 of lactic acid to glycolic acid, e.g., a ratio of about 50:50 of lactic acid to glycolic acid.

In some embodiments, the hydrophobic polymers of a) have a weight average molecular weight of from about 6 to about 12 kDa, for example from about 8 to about 10 kDa. In other embodiments, the hydrophobic polymers of a) have a weight average molecular weight of from about 4 to about 8 kDa. In some embodiments, the hydrophobic polymers of a) have a weight average molecular weight of from about 10 to about 100 kDa.

In some embodiments, at least a portion of the hydrophobic polymers of a) are covalently attached to a therapeutic peptide or protein and a portion of the hydrophobic polymers of a) are attached to a plurality of therapeutic peptides or proteins.

In some embodiments, at least a portion of the hydrophobic polymers of a) are coupled with a moiety that can dampen the pH of the hydrophobic polymer or particle. Exemplary pH dampening moieties include weakly basic salts such as calcium carbonate, magnesium hydroxide, and zinc carbonate, and proton sponges (e.g., including one or more amine groups) such as a polyamine.

In some embodiments, the hydrophilic-hydrophobic polymers of b) are block co-polymers. Exemplary block copolymers include a neutral hydrophilic block (e.g., which can enhance circulation), and a pH-responsive block (e.g., which can promote endosomal escape). Exemplary pH responsive blocks include those having a cis-acetonityl, hydrazone, or acetal linker, which can be hydrolyzed, for example, from pH 4 to 6.5. In some embodiment, the polymer includes a reversible peptide conjugation site, for example, which can provide means for peptide release from the carrier when reaching the cytosol (e.g., a thiol). In some embodiments, the hydrophilic-hydrophobic polymers of b) are di-block co-polymers (e.g., PEG-PLGA). In some embodiments, the hydrophilic-hydrophobic polymers of b) are tri-block-co-polymer (e.g., PEG-PLGA-PEG).

In some embodiments, the hydrophobic portion of at least a portion of the hydrophilic-hydrophobic polymers of b) has a hydroxyl terminal end. In some embodiments, the hydrophobic portion of at least a portion of the hydrophilic-hydrophobic polymers of b) have a hydroxyl terminal end and the hydroxyl terminal end is capped (e.g., capped with an acyl moiety). For example, in some embodiments, the hydrophobic portion of at least a portion of the hydrophilic-hydrophobic polymers of b) have a hydroxyl terminal end and the hydroxyl terminal end is capped with an acyl moiety.

In some embodiments, the hydrophobic portion of the hydrophilic-hydrophobic polymers of b) comprises copolymers of lactic and glycolic acid (i.e., PLGA). In some embodiments, the hydrophobic portion of the hydrophilic-hydrophobic polymers of b) comprises PLGA having a ratio of from about 15:85 or 25:75 to about 75:25 or 85:15 of lactic acid to glycolic acid, e.g., a ratio of about 50:50 of lactic acid to glycolic acid.

In some embodiments, the hydrophilic portion of the hydrophilic-hydrophobic polymers of b) has a weight average molecular weight of from about 1 to about 6 kDa (e.g., from about 2 to about 6 kDa). In some embodiments, the hydrophobic portion of the hydrophilic-hydrophobic polymers of b) has a weight average molecular weight of from about 8 to about 13 kDa.

In some embodiments, the plurality of hydrophilic-hydrophobic polymers of b) is from about 5 to about 25 weight % of said particle (e.g., from about 10 to about 25 weight %).

In some embodiments, the hydrophilic portion of the hydrophilic-hydrophobic polymers of b) comprises PEG.

In some embodiments, the hydrophilic portion of said hydrophilic-hydrophobic polymer terminates in a methoxy.

In some embodiments, the hydrophilic-hydrophobic polymers of b) have one or more of the following properties:

i) the hydrophilic portion has a weight average molecular weight of about 1-6 kDa (e.g., 2-6 kDa),

ii) the hydrophobic polymer has a weight average molecular weight of about 4-15 kDa;

iii) the hydrophilic polymer is PEG;

iv) the hydrophobic portion of the hydrophilic-hydrophobic polymer is made up of a first and a second type of monomeric subunit, and the ratio of the first to second type of monomeric subunit in the hydrophobic portion is from about 15:85 or 25:75 to about 75:25 or 85:15, e.g., about 50:50; and

v) the hydrophobic portion of the hydrophilic-hydrophobic polymer is PLGA.

In some embodiments, if the weight average molecular weight of the hydrophilic portion of the hydrophilic-hydrophobic polymer is about 1-3 kDa, e.g., about 2 kDa, the ratio of the weight average molecular weight of the hydrophilic portion to the weight average molecular weight of the hydrophobic portion is between 1:3-1:7, and if the weight average molecular weight of the hydrophilic portion is about 4-6 kDa, e.g., about 5 kDa, the ratio of the weight average molecular weight of the hydrophilic portion to the weight average molecular weight of the hydrophobic portion is between 1:1-1:4.

In some embodiments, the hydrophilic portion of the hydrophilic-hydrophobic polymer has a weight average molecular weight of about 2-6 kDa and the hydrophobic portion has a weight average molecular weight of between about 8-13 kDa.

In some embodiments, hydrophilic portion of said hydrophilic-hydrophobic polymer terminates in a methoxy,

In some embodiments, the therapeutic peptide is a therapeutic peptide described herein. In some embodiments, the therapeutic peptide comprises from about 2 to about 50 amino acid residues, e.g., about 2 to about 40 amino acid residues or about 2 to about 30 amino acid residues.

In some embodiments, the protein is a protein described herein.

In some embodiments, at least a portion of the therapeutic peptide or protein is chemically modified.

In some embodiments, the plurality of therapeutic peptides or proteins are from about 1 to about 90 weight % of said particle (e.g., from about 50% to about 90%, from about 70% to about 90%, from about 20% to about 70%).

In some embodiments, the particle further comprises a surfactant. In some embodiments, the surfactant is a polymer, e.g., the surfactant is PVA. In some embodiments, the PVA has a weight average molecular weight of from about 23 to about 26 kDa. In some embodiments, the surfactant is from about 15 to about 35 weight % of said particle.

In some embodiments, the particle further comprises a counterion. For example, in embodiments where the therapeutic peptide is a charged peptide, the particle can include a counterion, wherein the counterion has a charge opposite to that of the charge on the therapeutic peptide. In some embodiments, the ratio of the charge of the therapeutic peptide or protein to the charge of the counterion in the particle is from about 1:1.5 to about 1.5:1 (e.g., from about 1.25:1 to about 1:1.25, or about 1:1).

In some embodiments, the counterion can act as a surfactant (e.g., a single moiety can function as both a counterion and also a surfactant).

In some embodiments, the diameter of the particle is less than about 200 nm (e.g., less than about 150 nm).

In some embodiments, the surface of the particle is substantially coated with a polymer such as PEG.

In some embodiments, the zeta potential of the particle is from about −10 to about 10 mV (e.g., from about −5 to about 5 mV).

In some embodiments, the particle is chemically stable under conditions, comprising a temperature of 23 degrees Celsius and 60% percent humidity for at least 1 day (e.g., at least 7 days, at least 14 days, at least 21 days, at least 30 days).

In some embodiments, the particle is a lyophilized particle.

In some embodiments, the particle is formulated into a pharmaceutical composition.

In some embodiments, the surface of the particle is substantially free of a targeting agent.

In some aspects, the disclosure features a composition comprising a plurality of the particles described herein. In some embodiments, the composition is a pharmaceutical composition.

In some embodiments, at least 50%, 60%. 70%, 80%, 90%, 95%, 99% or all of the particles have a diameter of less than about 200 nM.

In some embodiments, the particles have a diameter a Dv90 of less than 200 nm (e.g., less than 150 nm).

In some embodiments, the composition is substantially free of polymers having a molecular weight of less than about 500 Da.

In some embodiments, the composition is substantially free of free therapeutic peptides or proteins (i.e., a therapeutic peptide or protein that is not embedded in or attached to the particles).

In some embodiments, the composition is chemically stable under ambient conditions for at least 1 day (e.g., at least 7 days, at least 14 days, at least 21 days, at least 30 days). In some embodiments, the composition is chemically stable under conditions comprising a temperature of 23 degrees Celsius and 60, 70, or 80 percent humidity for at least 1 day (e.g., at least 7 days, at least 14 days, at least 21 days, at least 30 days).

In some embodiments, the composition is a lyophilized composition.

In some embodiments, the composition, when administered to a subject, results in an AUC that is increased by at least 10, 20, 50, 75, 80, 90, 100, 200, or 500%, over the AUC for the therapeutic peptide or protein administered free (i.e., not in a particle) to the subject. In some embodiments, the composition and therapeutic peptide or protein administered free are administered under similar conditions. In some embodiments, the amount of therapeutic peptide or protein in the particle composition administered to the subject is the same, e.g., in terms of weight or number of molecules, as the amount of therapeutic peptide administered free. In some embodiments, the curve that defines the AUC is selected from:

a) a plot of the therapeutic peptide or protein in a selected target compartment, e.g., a selected tissue, organ or other compartment, vs. time.

In some embodiments, the curve is a plot of the therapeutic peptide or protein in a selected target compartment, e.g., peripheral blood vs. time. In some embodiments, AUC is calculated over a time period of 30 minutes, 1 hour, 2 hours, 3 hours, 6 hours, 12 hours, 24 hours, 2 days, or 7 days. In some embodiments, the time period begins at the time of, or 1 minute, 10 minutes, 60 minutes, 2 hours, 12 hours 24 hours, 2 days or 7 days after, administration of a dose of said composition or free therapeutic peptide or protein.

In some embodiments, the subject is any of a mouse, rat, dog, or human.

In some embodiments, the composition, when administered to a subject, results in a peak plasma concentration (Cₘₐₓ) that is less than 90, 80, 70, 60, 50, 40, 30, 20, 10, 5, or 1% of that of the Cₘₐₓ of said therapeutic peptide or protein administered free to the subject. In some embodiments, the composition and therapeutic peptide or protein administered free are administered under similar conditions. In some embodiments, the amount of therapeutic peptide or protein in the particle composition administered to the subject is the same, e.g., in terms of weight or number of molecules, as the amount administered free. In some embodiments, the Cₘₐₓ is measured by the presence of free labeled therapeutic peptide or protein in the plasma. In some embodiments, the Cₘₐₓ measurement(s) are taken over a time period of 30 minutes, 1 hour, 2 hours, 3 hours, 6 hours, 12 hours, 24 hours, 2 days, or 7 days. In some embodiments, the time period begins at the time of, or 1 minute, 10 minutes, 60 minutes, 2 hours, 12 hours 24 hours, 2 days or 7 days after, administration of a dose of the composition or therapeutic peptide or protein. In some embodiments, the subject is any of a mouse, rat, dog, or human.

In some embodiments, the composition, when administered to a subject, results in a volume of distribution (V_{z}) that is less than 90, 80, 70, 60, 50, 40, 30, 20, 10, 5, or 1% of that the V_{z} of the therapeutic peptide or protein administered free to the subject.

In some embodiments, the composition and therapeutic peptide or protein administered free are administered under similar conditions. In some embodiments, the amount of therapeutic peptide or protein in the particle composition administered to the subject is the same, e.g., in terms of weight or number of molecules, as the amount administered free. In some embodiments, V_{z} is measured by detecting free labeled therapeutic peptide or protein in the plasma. In some embodiments, V_{z} measurement(s) are taken over a time period of 30 minutes, 1 hour, 2 hours, 3 hours, 6 hours, 12 hours, 24 hours, 2 days, or 7 days. In some embodiments, the time period begins at the time of, or 1 minute, 10 minutes, 60 minutes, 2 hours, 12 hours 24 hours, 2 days or 7 days after, administration of a dose of the composition or free therapeutic peptide or protein. In some embodiments, the subject is any of a mouse, rat, dog, or human.

In some aspects, the disclosure features a kit comprising a plurality of particles described herein or a composition described herein.

In some aspects, the disclosure features a single dosage unit comprising a plurality of particles described herein or a composition described herein.

In some aspects, the disclosure features a method of treating a subject having a disorder comprising administering to said subject an effective amount of particles described herein or a composition described herein.

In one embodiment, the disorder is a proliferative disorder, e.g., a cancer, in a subject, e.g., a human, the method comprises: administering a composition that comprises a conjugate or particle described herein to a subject in an amount effective to treat the disorder, to thereby treat the proliferative disorder. In one embodiment, the composition is administered in combination with one or more additional anticancer agent, e.g., chemotherapeutic agent, e.g., a chemotherapeutic agent or combination of chemotherapeutic agents described herein, and radiation.

In one embodiment, the cancer is a cancer described herein. For example, the cancer can be a cancer of the bladder (including accelerated and metastatic bladder cancer), breast (e.g., estrogen receptor positive breast cancer; estrogen receptor negative breast cancer; HER-2 positive breast cancer; HER-2 negative breast cancer; progesterone receptor positive breast cancer; progesterone receptor negative breast cancer; estrogen receptor negative, HER-2 negative and progesterone receptor negative breast cancer (i.e., triple negative breast cancer); inflammatory breast cancer), colon (including colorectal cancer), kidney (e.g., transitional cell carcinoma), liver, lung (including small and non-small cell lung cancer, lung adenocarcinoma and squamous cell cancer), genitourinary tract, e.g., ovary (including fallopian tube and peritoneal cancers), cervix, prostate, testes, kidney, and ureter, lymphatic system, rectum, larynx, pancreas (including exocrine pancreatic carcinoma), esophagus, stomach, gall bladder, thyroid, skin (including squamous cell carcinoma), brain (including glioblastoma multiforme), head and neck (e.g., occult primary), and soft tissue (e.g., Kaposi's sarcoma (e.g., AIDS related Kaposi's sarcoma), leiomyosarcoma, angiosarcoma, and histiocytoma). Preferred cancers include breast cancer (e.g., metastatic or locally advanced breast cancer), prostate cancer (e.g., hormone refractory prostate cancer), renal cell carcinoma, lung cancer (e.g., non-small cell lung cancer, small cell lung cancer, lung adenocarcinoma, and squamous cell cancer, e.g., unresectable, locally advanced or metastatic non-small cell lung cancer, small cell lung cancer, lung adenocarcinoma, and squamous cell cancer), pancreatic cancer, gastric cancer (e.g., metastatic gastric adenocarcinoma), colorectal cancer, rectal cancer, squamous cell cancer of the head and neck, lymphoma (Hodgkin's lymphoma or non-Hodgkin's lymphoma), renal cell carcinoma, carcinoma of the urothelium, soft tissue sarcoma (e.g., Kaposi's sarcoma (e.g., AIDS related Kaposi's sarcoma), leiomyosarcoma, angiosarcoma, and histiocytoma), gliomas, myeloma (e.g., multiple myeloma), melanoma (e.g., advanced or metastatic melanoma), germ cell tumors, ovarian cancer (e.g., advanced ovarian cancer, e.g., advanced fallopian tube or peritoneal cancer), and gastrointestinal cancer.

In one embodiment, the disease or disorder associated with inflammation is a disease or disorder described herein. For example, the disease or disorder associated with inflammation can be for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, schleroderma, psoriasis, and dermatosis with acute inflammatory components.

In another embodiment, a composition comprising a particle or conjugate described herein may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The particle or conjugate described herein may be used to treat chronic hepatitis infection, including hepatitis B and hepatitis C.

Additionally, a composition comprising a particle or conjugate described herein may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

In one embodiment, the disorder is associated with cardiovascular disease, e.g., heart disease, in a subject, e.g., a human, the method comprises: administering a composition that comprises a particle or conjugate described herein to a subject in an amount effective to treat the disorder, to thereby treat the cardiovascular disease.

In one embodiment, cardiovascular disease is a disease or disorder described herein. For example, the cardiovascular disease may be cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using the particles, conjugates, compositions and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. Yet other disorders that may be treated with the particles, conjugates, compositions and methods described herein include restenosis, e.g., following coronary intervention, and disorders relating to an abnormal level of high density and low density cholesterol.

In one embodiment, a composition comprising a particle or conjugate described herein is administered to a subject undergoing or who has undergone angioplasty. In one embodiment, a composition comprising a particle or conjugate described herein is administered to a subject undergoing or who has undergone angioplasty with a stent placement. In some embodiments, a composition comprising a particle or conjugate described herein is can be used as a strut of a stent or a coating for a stent.

In one embodiment, the disorder is associated with the kidney, e.g., renal disorders, in a subject, e.g., a human, the method comprises: administering a composition that comprises a particle or conjugate described herein to a subject in an amount effective to treat the disorder, to thereby treat the disease or disorder associated with kidney disease.

In one embodiment, the disease or disorder associated with the kidney is a disease or disorder described herein. For example, the disease or disorder associated with the kidney can be for example, acute kidney failure, acute nephritic syndrome, analgesic nephropathy, atheroembolic renal disease, chronic kidney failure, chronic nephritis, congenital nephrotic syndrome, end-stage renal disease, goodpasture syndrome, interstitial nephritis, kidney damage, kidney infection, kidney injury, kidney stones, lupus nephritis, membranoproliferative GN I, membranoproliferative GN II, membranous nephropathy, minimal change disease, necrotizing glomerulonephritis, nephroblastoma, nephrocalcinosis, nephrogenic diabetes insipidus, nephrosis (nephrotic syndrome), polycystic kidney disease, post-streptococcal GN, reflux nephropathy, renal artery embolism, renal artery stenosis, renal papillary necrosis, renal tubular acidosis type I, renal tubular acidosis type II, renal underperfusion, renal vein thrombosis.

In some aspects, the disclosure features a therapeutic peptide or protein-hydrophobic polymer conjugate comprising a therapeutic peptide or protein covalently attached to a hydrophobic polymer, e.g., the therapeutic peptide or protein is covalently attached to the hydrophobic polymer via the carboxy terminal, the therapeutic peptide or protein is covalently attached to the hydrophobic polymer via the amino terminal and/or the therapeutic peptide or protein is covalently attached to the hydrophobic polymer via an amino acid side chain.

In some embodiments, the therapeutic peptide or protein is covalently attached to the hydrophobic polymer at a terminal end of the polymer.

In some embodiments, the therapeutic peptide or protein is covalently attached to the polymer on the backbone of the hydrophobic polymer.

In some embodiments, single therapeutic peptide or protein is covalently attached to a single hydrophobic polymer. In other embodiments, a plurality of therapeutic peptides or proteins are covalently attached to a single hydrophobic polymer.

In some embodiments, the therapeutic peptide or protein is directly covalently attached to the hydrophobic polymer (e.g., via an amide bond). In some embodiments, the therapeutic peptide or protein is covalently attached to the hydrophobic polymer via a linker. Exemplary linkers include a linker that comprises a moiety formed using “click chemistry” (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the therapeutic peptide or protein does not need to be cleaved to be active, e.g., the length of the linker is at least about 20 angstroms (e.g., at least about 24 angstroms).

In some embodiments, the hydrophobic polymer has a terminal hydroxyl moiety. In some embodiments, the hydroxy terminal end of the hydrophobic polymer is modified (e.g., by reacting with a functional moiety). In some embodiments, the hydrophobic polymer has a terminal hydroxyl moiety that is capped (e.g., with an acyl moiety).

In some embodiments, the hydrophobic polymer has a terminal carboxy moiety. In some embodiments, the carboxy terminal end of the hydrophobic polymer is modified (e.g., by reacting with a functional moiety).

In some embodiments, the hydrophobic polymer of the therapeutic peptide or protein—hydrophobic polymer conjugate has one or more of the following properties:

i) the hydrophobic polymer attached to the therapeutic peptide or protein be a homopolymer or a polymer made up of more than one kind of monomeric subunit;

ii) the hydrophobic polymer attached to the therapeutic peptide or protein has a weight average molecular weight of about 4-15 kDa (e.g., 6-12 kDa, 8-10 kDa);

iii) the hydrophobic polymer is made up of a first and a second type of monomeric subunit, and the ratio of the first to second type of monomeric subunit in the hydrophobic polymer attached to the therapeutic peptide or protein is from about 15:85 or 25:75 to about 75:25 or 85:15, e.g., about 50:50; and

iv) the hydrophobic polymer is PLGA.

In some aspects, the disclosure features a composition comprising a plurality of therapeutic peptide or protein-hydrophobic polymer conjugates described herein. In some embodiments, the composition is a pharmaceutical composition. In some embodiments, the composition is a reaction mixture.

In some embodiments, the composition is substantially free of un-conjugated therapeutic peptide or protein.

In some embodiments, the composition is substantially free of hydrophobic polymer having a molecular weight of less than about 500 Da.

In some aspects, the disclosure features a method of making a therapeutic peptide or protein-hydrophobic polymer conjugate described herein, the method comprising:

providing a therapeutic peptide or protein and a polymer; and

subjecting the therapeutic peptide or protein and the polymer to conditions that effect the covalent attachment of the therapeutic peptide or protein to the polymer.

In some embodiments, the method is performed in a reaction mixture, e.g., a reaction mixture comprising a single solvent or a reaction mixture comprising a solvent system of a plurality of solvents (e.g., the plurality of solvents are miscible, the solvent system comprises water and a polar solvent (e.g., DMF, DMSO, acetone, or acetonitrile), or the solvent system is bi-phasic (e.g., comprises an organic and aqueous phase)).

In some embodiments, the polymer is attached to an insoluble substrate.

In some embodiments, the method comprises the formation of a bond using “click chemistry” (e.g., as described in WO 2006/115547).

In some embodiments, the method results in the formation of an amide bond, a disulfide bond, an ester bond, and/or a triazole.

In some embodiments, the hydrophobic polymer has an aqueous solubility of less than about 1 mg/ml.

In some embodiments, the hydrophobic polymer is covalently attached the therapeutic peptide or protein through the amino terminal of the therapeutic peptide or protein. In some embodiments, the hydrophobic polymer is covalently attached the therapeutic peptide or protein through the carboxy terminal of the therapeutic peptide or protein. In some embodiments, the hydrophobic polymer is covalently attached the therapeutic peptide or protein through an amino acid side chain of the therapeutic peptide or protein.

In some embodiments, the therapeutic peptide or protein is covalently attached to the polymer at a terminal end of the hydrophobic polymer.

In some embodiments, the hydrophobic polymer has a hydroxyl and/or a carboxylic acid terminal end.

In some embodiments, the therapeutic peptide or protein is covalently attached to the polymer on the backbone of the hydrophobic polymer.

In some embodiments, a single therapeutic peptide or protein is covalently attached to a single hydrophobic polymer. In other embodiments, a plurality of therapeutic peptides or proteins are covalently attached to a single hydrophobic polymer.

In some embodiments, the method results in therapeutic peptide or protein-hydrophobic polymer conjugate having a purity of at least about 80% (e.g., at least about 85%, at least about 90%, at least about 95%, at least about 99%).

In some embodiments, the method produces at least about 100 mg of the therapeutic peptide or protein-hydrophobic polymer conjugate (e.g., at least about 1 g).

In some aspects, the disclosure features a therapeutic peptide or protein-hydrophobic polymer conjugate made by a method described herein.

In some aspects, the disclosure features a therapeutic peptide or protein-hydrophilic-hydrophobic polymer conjugate comprising a therapeutic peptide or protein covalently attached to a hydrophilic-hydrophobic polymer, wherein the hydrophilic-hydrophobic polymer comprises a hydrophilic portion attached to a hydrophobic portion.

In some embodiments, the therapeutic peptide or protein is attached to the hydrophilic portion of the hydrophilic-hydrophobic polymer.

In some embodiments, the therapeutic peptide is attached to the hydrophobic portion of the hydrophilic-hydrophobic polymer.

In some embodiments, the hydrophilic-hydrophobic polymer is covalently attached the therapeutic peptide or protein through the amino terminal of the therapeutic peptide or protein, the hydrophilic-hydrophobic polymer is covalently attached the therapeutic peptide or protein through the carboxy terminal of the therapeutic peptide or protein and/or the hydrophilic-hydrophobic polymer is covalently attached the therapeutic peptide or protein through an amino acid side chain of the therapeutic peptide or protein.

In some embodiments, the therapeutic peptide or protein is covalently attached to the hydrophilic-hydrophobic polymer at a terminal end of the polymer. In some embodiments, the therapeutic peptide or protein is covalently attached to the polymer on the backbone of the hydrophilic-hydrophobic polymer.

In some embodiments, a single therapeutic peptide or protein is covalently attached to a single hydrophilic-hydrophobic polymer.

In some embodiments, a plurality of therapeutic peptides or proteins are covalently attached to a single hydrophilic-hydrophobic polymer, e.g., a therapeutic peptide or protein is attached to the hydrophilic portion of the hydrophilic-hydrophobic polymer and a therapeutic peptide or protein is attached to the hydrophobic portion of the hydrophilic-hydrophobic polymer.

In some embodiments, the therapeutic peptide or protein is directly covalently attached to the hydrophobic portion of the hydrophobic-hydrophobic polymer (e.g., via an amide or ester bond).

In some embodiments, the therapeutic peptide or protein is directly covalently attached to the hydrophilic portion of the hydrophilic-hydrophobic polymer (e.g., via an amide or ester bond).

In some embodiments, the therapeutic peptide or protein is attached to the hydrophilic-hydrophobic polymer via a linker. Exemplary linkers include a linker that comprises a moiety formed using “click chemistry” (e.g., as described in WO 2006/115547) and a linker that comprises an amide, an ester, a disulfide, a sulfide, a ketal, a succinate, an oxime, a carbamate, a carbonate, a silyl ether, or a triazole (e.g., an amide, an ester, a disulfide, a sulfide, a ketal, a succinate, or a triazole). In some embodiments, the linker comprises a functional group such as a bond that is cleavable under physiological conditions. In some embodiments, the linker comprises a plurality of functional groups such as bonds that are cleavable under physiological conditions. In some embodiments, the linker includes a functional group such as a bond or functional group described herein that is not directly attached either to a first or second moiety linked through the linker at the terminal ends of the linker, but is interior to the linker. In some embodiments, the linker is hydrolysable under physiologic conditions, the linker is enzymatically cleavable under physiological conditions, or the linker comprises a disulfide which can be reduced under physiological conditions. In some embodiments, the linker is not cleaved under physiological conditions, for example, the linker is of a sufficient length that the therapeutic peptide or protein does not need to be cleaved to be active, e.g., the length of the linker is at least about 20 angstroms (e.g., at least about 24 angstroms).

In some embodiments, the hydrophilic-hydrophobic polymer have one or more of the following properties:

i) the hydrophilic portion has a weight average molecular weight of about 1-6 kDa (e.g., 2-6 kDa),

ii) the hydrophobic polymer has a weight average molecular weight of about 4-15 kDa (e.g., 6-12 kDa, 8-10 kDa);

iii) the hydrophilic polymer is PEG;

iv) the hydrophobic polymer is made up of a first and a second type of monomeric subunit, and the ratio of the first to second type of monomeric subunit in the hydrophobic polymer attached to the therapeutic peptide is from about 15:85 or 25:75 to about 75:25 or 85:15, e.g., about 50:50; and

v) the hydrophobic polymer is PLGA.

In some embodiments, if the weight average molecular weight of the hydrophilic portion of the hydrophilic-hydrophobic polymer is about 1-3 kDa, e.g., about 2 kDa, the ratio of the weight average molecular weight of the hydrophilic portion to the weight average molecular weight of the hydrophobic portion is between 1:3-1:7, and if the weight average molecular weight of said hydrophilic portion is about 4-6 kDa, e.g., about 5 kDa, the ratio of the weight average molecular weight of said hydrophilic portion to the weight average molecular weight of said hydrophobic portion is between 1:1-1:4.

In some embodiments, the hydrophilic portion of the hydrophilic-hydrophobic polymer has a weight average molecular weight of about 2-6 kDa and the hydrophobic portion has a weight average molecular weight of between about 8-13 kDa.

In some embodiments, the hydrophilic portion of the hydrophilic-hydrophobic polymer terminates in a methoxy.

In some aspects, the disclosure features a composition comprising a plurality of therapeutic peptide or protein-hydrophilic-hydrophobic polymer conjugates described herein. In some embodiments, the composition is a pharmaceutical composition. In some embodiments, the composition is a reaction mixture.

In some embodiments, the composition is substantially free of un-conjugated therapeutic peptide.

In some embodiments, the composition is substantially free of hydrophilic-hydrophobic polymer having a molecular weight of less than about 500 Da.

In some aspects, the disclosure features a method of making a therapeutic peptide or protein-hydrophilic-hydrophobic polymer conjugate described herein, the method comprising:

providing a therapeutic peptide or protein and a hydrophilic-hydrophobic polymer; and

subjecting the therapeutic peptide or protein and hydrophilic-hydrophobic polymer to conditions that effect the covalent attachment of the therapeutic peptide or protein to the polymer.

In some embodiments, the method is performed in a reaction mixture, e.g., the reaction mixture comprises a single solvent or the reaction mixture comprises a solvent system comprising a plurality of solvents (e.g., the plurality of solvents are miscible or the solvent system is bi-phasic (e.g., comprises an organic and aqueous phase)).

In some embodiments, at least one of the therapeutic peptide, protein or hydrophilic-hydrophobic polymer is attached to an insoluble substrate, e.g., the hydrophilic-hydrophobic polymer is attached to an insoluble substrate.

In some embodiments, the method comprises the formation of a bond using “click chemistry” (e.g., as described in WO 2006/115547).

In some embodiments, the method results in the formation of an amide bond, a disulfide bond, an ester bond, and/or a tetrazole.

In some embodiments, the hydrophilic-hydrophobic polymer is covalently attached the therapeutic peptide through the amino terminal of the therapeutic peptide or protein, the hydrophilic-hydrophobic polymer is covalently attached the therapeutic peptide or protein through the carboxy terminal of the therapeutic peptide or protein and/or the hydrophilic-hydrophobic polymer is covalently attached the therapeutic peptide or protein through an amino acid side chain of the therapeutic peptide or protein.

In some embodiments, the therapeutic peptide or protein is covalently attached to the hydrophobic-hydrophilic polymer at a terminal end of the polymer.

In some embodiments, the therapeutic peptide or protein is covalently attached to the hydrophobic-hydrophilic polymer on the hydrophilic portion of the polymer. In some embodiments, the therapeutic peptide or protein is covalently attached to the hydrophobic-hydrophilic polymer on the hydrophobic portion of the polymer. In some embodiments, the therapeutic peptide or protein is covalently attached to the hydrophobic-hydrophilic polymer on the backbone of the polymer.

In some embodiments, a single therapeutic peptide or protein is covalently attached to a single hydrophobic-hydrophilic polymer.

In some embodiments, a plurality of therapeutic peptides or proteins is covalently attached to a single hydrophobic-hydrophilic polymer. In some embodiments, the therapeutic peptide or protein is covalently attached to the hydrophobic-hydrophilic polymer on the hydrophilic portion of the polymer, the therapeutic peptide or protein is covalently attached to the hydrophobic-hydrophilic polymer on the hydrophobic portion of the polymer and/or the therapeutic peptide or protein is covalently attached to the hydrophobic-hydrophilic polymer on the backbone of the polymer

In some embodiments, the method results in a therapeutic peptide or protein-hydrophilic-hydrophobic polymer conjugate having a purity of at least about 80% (e.g., at least about 85%, at least about 90%, at least about 95%, at least about 99%).

In some embodiments, the method produces at least about 100 mg of the therapeutic peptide or protein-hydrophobic polymer conjugate (e.g., at least about 1 g).

In some aspects, the disclosure features a therapeutic peptide or protein-hydrophilic-hydrophobic polymer conjugate made by a method described herein.

In another aspect, the invention features, a method of storing a conjugate, particle or composition, the method comprising:

providing said conjugate, particle or composition disposed in a container, e.g., an air or liquid tight container, e.g., a container described herein, e.g., a container having an inert gas, e.g., argon or nitrogen, filled headspace;

storing said conjugate, particle or composition, e.g., under preselected conditions, e.g., temperature, e.g., a temperature described herein;

and, moving said container to a second location or removing all or an aliquot of said conjugate, particle or composition, from said container.

In an embodiment the conjugate, particle or composition is evaluated, e.g., for stability or activity of the therapeutic peptide or protein, a physical property, e.g., color, clumping, ability to flow or be poured, or particle size or charge. The evaluation can be compared to a standard, and optionally, responsive to said standard, the conjugate, particle or composition, is classified.

In an embodiment, a conjugate, particle or composition is stored as a re-constituted formulation (e.g., in a liquid as a solution or suspension).

In one aspect, a protein can be used instead of a therapeutic peptide in any of the aspects and embodiments described above. A “protein”, as used herein, has more than 100 amino acids or more, e.g., the protein is at least 110 amino acids in length.

### BRIEF DESCRIPTION OF THE FIGURES

FIGS. 1A-C describes exemplary linkers which may be used to attach moieties described herein.

### DETAILED DESCRIPTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Particles, conjugates (e.g., therapeutic peptide-polymer conjugates, and protein-polymer conjugates) and compositions are described herein. Also disclosed are dosage forms containing the conjugates, particles and compositions; methods of using the conjugates, particles and compositions (e.g., to treat a disorder); kits including the conjugates, particles and compositions; methods of making the conjugates, particles and compositions; methods of storing the conjugates, particles and compositions; and methods of analyzing the conjugates, particles and compositions.

Headings, and other identifiers, e.g., (a), (b), (i) etc, are presented merely for ease of reading the specification and claims. The use of headings or other identifiers in the specification or claims does not require the steps or elements be performed in alphabetical or numerical order or the order in which they are presented.

### DEFINITIONS

The term “ambient conditions,” as used herein, refers to surrounding conditions at about one atmosphere of pressure, 50% relative humidity and about 25° C., unless specified as otherwise.

The term “anionic moiety” refers to a moiety, which has a pKa of less than 3, 2, 1 or 0 and/or a negative charge in at least one of the following conditions: during the production of a particle described herein, when formulated into a particle described herein, or subsequent to administration of a particle described herein to a subject, for example, while circulating in the subject and/or while in the endosome. Anionic moieties include polymeric species, such as moieties having more than one charge.

The term “anionic polymer” refers to an anionic moiety that has a plurality of negative charges (i.e., at least 2 under at least 1 of the conditions described above), e.g., when formulated into a particle described herein. In some embodiments, the anionic polymer has at least 3, 4, 5, 10, 15, or 20 negative charges.

The term “attach,” as used herein with respect to the relationship of a first moiety to a second moiety, e.g., the attachment of a therapeutic peptide to a polymer, refers to the formation of a covalent bond between a first moiety and a second moiety. In the same context, the noun “attachment” refers to a covalent bond between the first and second moiety. For example, a therapeutic peptide attached to a polymer is a therapeutic peptide covalently bonded to the polymer (e.g., a hydrophobic polymer described herein). The attachment can be a direct attachment, e.g., through a direct bond of the first moiety to the second moiety, or can be through a linker (e.g., through a covalently linked chain of one or more atoms disposed between the first and second moiety). E.g., where an attachment is through a linker, a first moiety (e.g., a drug) is covalently bonded to a linker, which in turn is covalently bonded to a second moiety (e.g., a hydrophobic polymer described herein).

The term “biodegradable” includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to a side chain or that connects a side chain to the polymer backbone. In certain embodiments, one or the other or both general types of biodegradation may occur during use of a polymer.

The term “biodegradation,” as used herein, encompasses both general types of biodegradation described above. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of a polymer, assembly of polymers or particle, and the mode and location of administration. For example, a greater molecular weight, a higher degree of crystallinity, and/or a greater biostability, usually lead to slower biodegradation.

The term “cationic moiety” refers to a moiety, which has a pKa of 5 or greater (e.g., a Lewis base having a pKa of 5 or greater) and/or a positive charge in at least one of the following conditions: during the production of a particle described herein, when formulated into a particle described herein, or subsequent to administration of a particle described herein to a subject, for example, while circulating in the subject and/or while in the endosome. Exemplary cationic moieties include amine containing moieties (e.g., charged amine moieties such as a quaternary amine), guanidine containing moieties (e.g., a charged guanidine such as a quanadinium moiety), and heterocyclic and/or heteroaromatic moieties (e.g., charged moieties such as a pyridinium or a histidine moiety). Cationic moieties include polymeric species, such as moieties having more than one charge, e.g., contributed by repeated presence of a moiety, (e.g., a cationic PVA and/or a polyamine). Cationic moieties also include zwitterions, meaning a compound that has both a positive charge and a negative charge (e.g., an amino acid such as arginine, lysine, or histidine).

The term “cationic polymer,” for example, a polyamine, refers to a polymer (the term “polymer” is described below) that has a plurality of positive charges (i.e., at least 2 under at least one of the cond described above), e.g., when formulated into a particle described herein. In some embodiments, the cationic polymer, for example, polyamine, has at least 3, 4, 5, 10, 15, or 20 positive charges.

The phrase “cleavable under physiological conditions” refers to a bond having a half life of less than about 50 or less than about 100 hours, when subjected to physiological conditions. For example, enzymatic degradation can occur over a period of less than about five years, one year, six months, three months, one month, fifteen days, five days, three days, or one day upon exposure to physiological conditions (e.g., an aqueous solution having a pH from about 4 to about 8, and a temperature from about 25° C. to about 37° C.

An “effective amount” or “an amount effective” refers to an amount of the therapeutic peptide-polymer conjugate, particle or composition which is effective, upon single or multiple dose administrations to a subject, in treating a cell, or curing, alleviating, relieving or improving a symptom of a disorder. An effective amount of the therapeutic peptide-polymer conjugate, particle or composition may vary according to factors such as the disease state, age, sex, and weight of the individual, and the ability of the compound to elicit a desired response in the individual. An effective amount is also one in which any toxic or detrimental effects of the therapeutic peptide-polymer conjugate, particle or composition is outweighed by the therapeutically beneficial effects.

The term “embed,” as used herein, refers to disposing a first moiety with, or within, a second moiety by the formation of a non-covalent interaction between the first moiety and the second moiety, e.g., a therapeutic peptide and a polymer (e.g., a therapeutic or diagnostic agent and a hydrophobic polymer). In an embodiment, when referring to a moiety embedded in a particle, that moiety (e.g., a therapeutic peptide or a counterion) is associated with a polymer or other component of the particle through one or more non-covalent interactions such as van der Waals interactions, hydrophobic interactions, hydrogen bonding, dipole-dipole interactions, ionic interactions, pi stacking, and covalent bonds between the moieties and polymer or other components of the particle are absent. An embedded moiety may be completely or partially surrounded by the polymer or particle in which it is embedded.

The term “hydrophobic,” as used herein, describes a moiety that can be dissolved in an aqueous solution at physiological ionic strength only to the extent of less than about 0.05 mg/mL (e.g., about 0.01 mg/mL or less).

The term “hydrophilic,” as used herein, describes a moiety that has a solubility, in aqueous solution at physiological ionic strength, of at least about 0.05 mg/mL or greater.

The term “hydrophilc-hydrophobic polymer” as used herein, describes a polymer comprising a hydrophilic portion attached to a hydrophobic portion. Exemplary hydrophilic-hydrophobic polymers include block-copolymers, e.g., comprising a block of hydrophilic polymers and a block of hydrophobic polymers.

A “hydroxy protecting group” as used herein, is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable hydroxy protecting groups include, for example, acyl (e.g., acetyl), triethylsilyl (TES), t-butyldimethylsilyl (TBDMS), 2,2,2-trichloroethoxycarbonyl (Troc), and carbobenzyloxy (Cbz).

“Inert atmosphere,” as used herein, refers to an atmosphere composed primarily of an inert gas, which does not chemically react with the polymer-agent conjugates, particles, compositions or mixtures described herein. Examples of inert gases are nitrogen (N₂), helium, and argon.

“Linker,” as used herein, is a moiety that connects two or more moieties together (e.g., a therapeutic peptide or counterion and a polymer such as a hydrophobic or hydrophilic-hydrophobic, or hydrophilic polymer). Linkers have at least two functional groups. For example, a linker having two functional groups may have a first functional group capable of reacting with a functional group on a moiety such as a therapeutic peptide, a counterion, a hydrophobic moiety such as a polymer, or a hydrophilic-hydrophobic polymer described herein, and a second functional group capable of reacting with a functional group on a second moiety such as a therapeutic peptide, a counterion, a hydrophobic moiety such as a polymer, or a hydrophilic-hydrophobic polymer described herein.

A linker may have more than two functional groups (e.g., 3, 4, 5, 6, 7, 8, 9, 10 or more functional groups), which may be used, e.g., to link multiple agents to a polymer or to provide a biocleavable moiety within the linker. In some embodiments, for example, when a linker has more than two functional groups, e.g., the linker comprises a functional group in addition to the two functional groups connecting a first moiety to a second moiety, the additional functional group (e.g., a third functional group) can be positioned in between the first and second group, and in some embodiments, can be cleaved, for example, under physiological conditions. For example, a linker may be of the form

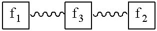

wherein f₁ is a first functional group, e.g., a functional group capable of reacting with a functional group on a moiety such as a therapeutic peptide or protein, a counterion, a hydrophobic moiety such as a polymer, e.g., a hydrophobic polymer described herein, or a hydrophilic-hydrophobic moiety, e.g., a hydrophilic-hydrophobic polymer described herein; f₂ is a second functional group, e.g., a functional group capable of reacting with a functional group on a second moiety such as a therapeutic peptide or protein described herein or a counterion described herein; f₃ is a biocleavable functional group, e.g., a biocleaveable bond described herein; and “ [symbol] ” represents a spacer connecting the functional groups, e.g., an alkylene (divalent alkyl) group wherein, optionally, one or more carbon atoms of the alkylene linker is replaced with one or more heteroatoms (e.g., resulting in one of the following groups: thioether, amino, ester, ether, keto, amide, silyl ether, oxime, carbamate, carbonate, disulfide, heterocyclic, or heteroaromatic). Depending on the context, linker can refer to a linker moiety before attachment to either of a first or second moiety (e.g., therapeutic peptide or polymer), after attachment to one moiety but before attachment to a second moiety, or the residue of the linker present after attachment to both the first and second moiety.

The term “lyoprotectant,” as used herein refers to a substance present in a lyophilized preparation. Typically it is present prior to the lyophilization process and persists in the resulting lyophilized preparation. Typically a lyoprotectant is added after the formation of the particles. If a concentration step is present, e.g., between formation of the particles and lyophilization, a lyoprotectant can be added before or after the concentration step. A lyoprotectant can be used to protect particles, during lyophilization, for example to reduce or prevent aggregation, particle collapse and/or other types of damage. In an embodiment the lyoprotectant is a cryoprotectant.

In an embodiment the lyoprotectant is a carbohydrate. The term “carbohydrate,” as used herein refers to and encompasses monosaccharides, disaccharides, oligosaccharides and polysaccharides.

In an embodiment, the lyoprotectant is a monosaccharide. The term “monosaccharide,” as used herein refers to a single carbohydrate unit (e.g., a simple sugar) that can not be hydrolyzed to simpler carbohydrate units. Exemplary monosaccharide lyoprotectants include glucose, fructose, galactose, xylose, ribose and the like.

In an embodiment, the lyoprotectant is a disaccharide. The term “disaccharide,” as used herein refers to a compound or a chemical moiety formed by 2 monosaccharide units that are bonded together through a glycosidic linkage, for example through 1-4 linkages or 1-6 linkages. A disaccharide may be hydrolyzed into two monosaccharides. Exemplary disaccharide lyoprotectants include sucrose, trehalose, lactose, maltose and the like.

In an embodiment, the lyoprotectant is an oligosaccharide. The term “oligosaccharide,” as used herein refers to a compound or a chemical moiety formed by 3 to about 15, preferably 3 to about 10 monosaccharide units that are bonded together through glycosidic linkages, for example through 1-4 linkages or 1-6 linkages, to form a linear, branched or cyclic structure. Exemplary oligosaccharide lyoprotectants include cyclodextrins, raffinose, melezitose, maltotriose, stachyose acarbose, and the like. An oligosaccharide can be oxidized or reduced.

In an embodiment, the lyoprotectant is a cyclic oligosaccharide. The term “cyclic oligosaccharide,” as used herein refers to a compound or a chemical moiety formed by 3 to about 15, preferably 6, 7, 8, 9, or 10 monosaccharide units that are bonded together through glycosidic linkages, for example through 1-4 linkages or 1-6 linkages, to form a cyclic structure. Exemplary cyclic oligosaccharide lyoprotectants include cyclic oligosaccharides that are discrete compounds, such as a cyclodextrin, β cyclodextrin, or γ cyclodextrin.

Other exemplary cyclic oligosaccharide lyoprotectants include compounds which include a cyclodextrin moiety in a larger molecular structure, such as a polymer that contains a cyclic oligosaccharide moiety. A cyclic oligosaccharide can be oxidized or reduced, for example, oxidized to dicarbonyl forms. The term “cyclodextrin moiety,” as used herein refers to cyclodextrin (e.g., an α, β, or γ cyclodextrin) radical that is incorporated into, or a part of, a larger molecular structure, such as a polymer. A cyclodextrin moiety can be bonded to one or more other moieties directly, or through an optional linker A cyclodextrin moiety can be oxidized or reduced, for example, oxidized to dicarbonyl forms.

Carbohydrate lyoprotectants, e.g., cyclic oligosaccharide lyoprotectants, can be derivatized carbohydrates. For example, in an embodiment, the lyoprotectant is a derivatized cyclic oligosaccharide, e.g., a derivatized cyclodextrin, e.g., 2 hydroxy propyl-beta cyclodextrin, e.g., partially etherified cyclodextrins (e.g., partially etherified β cyclodextrins) disclosed in U.S. Pat. No. 6,407,079, the contents of which are incorporated herein by this reference. Another example of a derivatized cyclodextrin is β-cyclodextrin sulfobutylether sodium.

An exemplary lyoprotectant is a polysaccharide. The term “polysaccharide,” as used herein refers to a compound or a chemical moiety formed by at least 16 monosaccharide units that are bonded together through glycosidic linkages, for example through 1-4 linkages or 1-6 linkages, to form a linear, branched or cyclic structure, and includes polymers that comprise polysaccharides as part of their backbone structure. In backbones, the polysaccharide can be linear or cyclic. Exemplary polysaccharide lyoprotectants include glycogen, amylase, cellulose, dextran, maltodextrin and the like.

The term “derivatized carbohydrate,” refers to an entity which differs from the subject non-derivatized carbohydrate by at least one atom. For example, instead of the —OH present on a non-derivatized carbohydrate the derivatized carbohydrate can have —OX, wherein X is other than H. Derivatives may be obtained through chemical functionalization and/or substitution or through de novo synthesis—the term “derivative” implies no process-based limitation.

In some embodiments, the lyoprotectant is a reduced sugar alcohol such as, e.g., mannitol.

The term “nanoparticle” is used herein to refer to a material structure whose size in at least any one dimension (e.g., x, y, and z Cartesian dimensions) is less than about 1 micrometer (micron), e.g., less than about 500 nm or less than about 200 nm or less than about 100 nm, and greater than about 5 nm. In embodiments, the size is less than about 70 nm but greater than about 20 nm. A nanoparticle can have a variety of geometrical shapes, e.g., spherical, ellipsoidal, etc. The term “nanoparticles” is used as the plural of the term “nanoparticle.”

As used herein, “particle polydispersity index (PDI)” or “particle polydispersity” refers to the width of the particle size distribution. Particle PDI can be calculated from the equation PDI=2a₂/a₁² where a₁ is the 1^{st} Cumulant or moment used to calculate the intensity weighted Z average mean size and a₂ is the 2^{nd} moment used to calculate a parameter defined as the polydispersity index (PdI). A particle PDI of 1 is the theoretical maximum and would be a completely flat size distribution plot. Compositions of particles described herein may have particle PDIs of less than 0.5, less than 0.4, less than 0.3, less than 0.2, or less than 0.1.

“Pharmaceutically acceptable carrier or adjuvant,” as used herein, refers to a carrier or adjuvant that may be administered to a patient, together with a polymer-agent conjugate, particle or composition described herein, and which does not destroy the pharmacological activity thereof and is nontoxic when administered in doses sufficient to deliver a therapeutic amount of the particle. Some examples of materials which can serve as pharmaceutically acceptable carriers include: (1) sugars, such as lactose, glucose, mannitol and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions.

The term “polymer,” as used herein, is given its ordinary meaning as used in the art, i.e., a molecular structure featuring one or more repeat units (monomers), connected by covalent bonds. The repeat units may all be identical, or in some cases, there may be more than one type of repeat unit present within the polymer. Polymers may be natural or unnatural (synthetic) polymers. Polymers may be homopolymers or copolymers containing two or more monomers. Polymers may be linear or branched.

If more than one type of repeat unit is present within the polymer, then the polymer is to be a “copolymer.” It is to be understood that in any embodiment employing a polymer, the polymer being employed may be a copolymer. The repeat units forming the copolymer may be arranged in any fashion. For example, the repeat units may be arranged in a random order, in an alternating order, or as a “block” copolymer, i.e., containing one or more regions each containing a first repeat unit (e.g., a first block), and one or more regions each containing a second repeat unit (e.g., a second block), etc. Block copolymers may have two (a diblock copolymer), three (a triblock copolymer), or more numbers of distinct blocks. In terms of sequence, copolymers may be random, block, or contain a combination of random and block sequences.

In some cases, the polymer is biologically derived, i.e., a biopolymer. Non-limiting examples of biopolymers include peptides or proteins (i.e., polymers of various amino acids), or nucleic acids such as DNA or RNA.

As used herein, “polymer polydispersity index (PDI)” or “polymer polydispersity” refers to the distribution of molecular mass in a given polymer sample. The polymer PDI calculated is the weight average molecular weight divided by the number average molecular weight. It indicates the distribution of individual molecular masses in a batch of polymers. The polymer PDI has a value typically greater than 1, but as the polymer chains approach uniform chain length, the PDI approaches unity (1).

As used herein, the term “prevent” or “preventing” as used in the context of the administration of an agent to a subject, refers to subjecting the subject to a regimen, e.g., the administration of a polymer-agent conjugate, particle or composition, such that the onset of at least one symptom of the disorder is delayed as compared to what would be seen in the absence of the regimen.

As used herein, the term “protein” refers to a plurality of linked amino acids that has 100 amino acids or more. For example, the protein can be 110, 120, 130, 140, 150, 160, 170, 180, 190, 200, 220, 240, 260, 280, 300, 320, 340, 360, 380, 400, 420, 440, 460, 480, 500 or more amino acids in length. Proteins include, for example, adapter proteins, antibodies, carbohydrate binding proteins, carrier proteins, cell cycle proteins, chemokines, chromosomal proteins, collagens, cytokines, fibrous proteins, growth factors, heat shock proteins, interferons, oncogene proteins, proteases, ubiquitins, zinc finger proteins, and fragments thereof.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dog, cat, cow, pig, etc.

The term “therapeutic peptide,” as used herein, refers to a peptide comprising two or more amino acids but not more than 100 amino acids, covalently linked together through one or more amide bonds, wherein upon administration of the peptide to a subject, the subject receives a therapeutic effect (e.g., administration of the therapeutic peptide treats a cell, or cures, alleviates, relieves or improves a symptom of a disorder) as opposed to, e.g., the use of a peptide as a linker which itself has no therapeutic effect. A therapeutic peptide may comprise, e.g., more than three, four, five, six, seven, eight, nine, ten, eleven, twelve, thirteen, fourteen, fifteen amino acids. In some embodiments, a therapeutic peptide comprises more than 15, e.g., greater than 20, 25, 30, 35, 40, 45, 50, 55, 60, 65, 70, 75, 80, 85, or 90 amino acids. For example, in some embodiments, the therapeutic peptide is more than 9, 10, 11 or 12 amino acids in length.

The therapeutic effect of the therapeutic peptide can occur by the therapeutic peptide acting as an agonist or as an antagonist. The term “agonist,” as used herein, is meant to refer to a peptide that mimics, or up-regulates, (e.g., potentiates or supplements) the activity of a protein. A direct agonist has at least one activity of the species to be agonized. E.g., a direct agonist can be a wild-type peptide or derivative thereof that has at least one activity of the wild-type protein. An indirect agonist can be a peptide which increases at least one activity of a protein. An indirect agonist includes a peptide which increases the interaction of a polypeptide with another molecule, e.g., a target peptide or nucleic acid. “Antagonist” as used herein is meant to refer to a peptide that reduces or down regulates (e.g., suppresses or inhibits) at least one activity of a protein. A direct antagonist can be a peptide which inhibits or decreases the interaction between a protein and another molecule, e.g., a target peptide or enzyme substrate. An indirect antagonist can be a peptide which reduces the amount of expressed protein present. In some embodiments, the therapeutic peptide is an agonist or an antagonist of a cytokine, a protease, a kinase or a membrane protein.

Exemplary therapeutic peptides include, e.g., a peptide that treats a cell, or cures, alleviates, relieves or improves a symptom of a metabolic disorder, e.g., a hormone, e.g., an anti-diabetogenic peptide; a peptide that treats a cell, or cures, alleviates, relieves or improves a symptom of a proliferative disorder, e.g., a tumor or metastases thereof; a peptide that treats a cell, or cures, alleviates, relieves or improves a symptom of a cardiovascular disorder; a peptide that treats a cell, or cures, alleviates, relieves or improves a symptom of an infectious disease; and a peptide that treats a cell, or cures, alleviates, relieves or improves a symptom of an allergic, inflammatory or autoimmune disorder. In some instances, the therapeutic peptide is not a hormone. For example, in some embodiments, the therapeutic peptide is a peptide other than luteinizing hormone releasing hormone (LHRH). In some embodiments, the therapeutic peptide is a peptide other than tubulysin. In some embodiments, the therapeutic peptide does not interact with, e.g., bind to an integrin. For example, in one embodiment, the therapeutic peptide does not have the sequence Arg-Gly-Asp.

Therapeutic peptides can comprise α-, β- and/or γ-amino acids. For example, the therapeutic peptide can comprise three or more α-amino acids, e.g., three or more consecutive α-amino acids. In one embodiment, the therapeutic peptide comprises at least four, five, six, seven, eight, nine, ten, or more α-amino acids, e.g., at least four, five, six, seven, eight, nine, ten, or more consecutive α-amino acids. Typically, all of the amino acids of the therapeutic peptide are α-amino acids or the therapeutic peptide includes less than 5, 4, 3 or 2 non-α amino acids. A therapeutic peptide may be linear, branched, cyclic, or a combination thereof.

In some instances, the therapeutic peptide is a “standard therapeutic peptide”, i.e., the majority of the amino acids (i.e., greater than 50% of the amino acids, e.g., 51%, 55%, 60%, 70%, 80%, 85%, 90%, 95%, 99%, or all of the amino acids) of the therapeutic peptide are standard amino acids. Standard amino acids are Ala, Arg, Asn, Asp, Cys, Gln, Glu, Gly, His, Ile, Leu, Lys, Met, Phe, Pro, Ser, Thr, Trp, Tyr, Val, Asx, and Glx. In other embodiments, the therapeutic peptide is a “non-standard therapeutic peptide”, i.e., the majority of the amino acids (i.e., greater than 50% of the amino acids, e.g., 51%, 55%, 60%, 70%, 80%, 85%, 90%, 95%, 99%, or all of the amino acids) of the therapeutic peptide are non-standard amino acids. The term “non-standard amino acid”, as used herein, refers to amino acids that have the required amino group, carboxylic acid, and side chain, but are not Ala, Arg, Asn, Asp, Cys, Gln, Glu, Gly, His, Ile, Leu, Lys, Met, Phe, Pro, Ser, Thr, Trp, Tyr, Val, Asx, or Glx.

The “therapeutic peptide” can be a fragment of a protein, e.g., a fragment having an amino acid sequence corresponding to the sequence of a known protein. In some embodiments, the therapeutic peptide is a fragment having an amino acid sequence corresponding to the sequence of a commercially available reference protein, and the glycan structure of the fragment differs from the glycan structure of the fragment from the commercially-available protein fragment. For example, the glycan structure of the therapeutic peptide may differ from the naturally-occurring glycosylation pattern of the peptide by one or more glycans, e.g., two, e.g., three, e.g., four, e.g., five, e.g., six, e.g., seven, e.g., eight, e.g., nine, e.g., ten or greater glycans.

In preferred embodiments, the therapeutic peptide is attached to the polymer via a linker (e.g., through a covalently linked chain of one or more atoms disposed between the therapeutic peptide or protein and the polymer). The linker can be, e.g., a linker described herein.

In an embodiment, the therapeutic peptide has no substantial effect on the localization of the particle, e.g., it does not target the particle by affinity to a ligand, e.g., a surface protein or extracellular matrix component.

In some embodiments, if the conjugate includes a targeting agent that is a peptide, the targeting agent is a peptide or protein that differs from the therapeutic peptide or protein.

As used herein, the term “treat” or “treating” a subject having a disorder refers to subjecting the subject to a regimen, e.g., the administration of a polymer-agent conjugate, particle or composition, such that at least one symptom of the disorder is cured, healed, alleviated, relieved, altered, remedied, ameliorated, or improved. Treating includes administering an amount effective to alleviate, relieve, alter, remedy, ameliorate, improve or affect the disorder or the symptoms of the disorder. The treatment may inhibit deterioration or worsening of a symptom of a disorder.

The term “zwitterionic moiety” refers to a moiety, which has both a positive and a negative charge in at least one of the following conditions: during the production of a particle described herein, when formulated into a particle described herein, or subsequent to administration of a particle described herein to a subject, for example, while circulating in the subject and/or while in the endosome. Zwitterionic moieties include polymeric species, such as moieties having more than one charge.

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents). Exemplary acyl groups include acetyl (CH₃C(O)—), benzoyl (C₆H₅C(O)—), and acetylamino acids (e.g., acetylglycine, CH₃C(O)NHCH₂C(O)—.

The term “alkoxy” refers to an alkyl group, as defined below, having an oxygen radical attached thereto. Representative alkoxy groups include methoxy, ethoxy, propyloxy, tert-butoxy and the like.

The term “carboxy” refers to a —C(O)OH or salt thereof.

The term “hydroxy” and “hydroxyl” are used interchangably and refer to —OH.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Any atom can be substituted. Suitable substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙ alkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). In one aspect, the substituents on a group are independently any one single, or any subset of the aforementioned substituents. In another aspect, a substituent may itself be substituted with any one of the above substituents.

Particles

The particles, in general, include a therapeutic peptide or protein, and at least one of a counterion, a hydrophobic moiety, such as a polymer, or a hydrophilic-hydrophobic polymer. In some embodiments, the particles include a therapeutic peptide or protein and a counterion, and at least one of a hydrophobic moiety, such as a polymer, or a hydrophilic-hydrophobic polymer. In some embodiments, a particle described herein includes a hydrophobic moiety such as a hydrophobic polymer or lipid (e.g., hydrophobic polymer), a polymer containing a hydrophilic portion and a hydrophobic portion, a therapeutic peptide or protein, and a counterion. In some embodiments, the therapeutic peptide or protein and/or counterion is attached to a moiety. For example, the therapeutic peptide or protein and/or counterion can be attached to a polymer (e.g., the hydrophobic polymer or the polymer containing a hydrophilic portion and a hydrophobic portion). In some embodiments, the therapeutic peptide or protein is attached to a polymer (e.g., a hydrophobic polymer or a polymer containing a hydrophilic and a hydrophobic portion), and the counterion is not attached to a polymer (e.g., the counterion is embedded in the particle). In some embodiments, the therapeutic peptide or protein and the counterion are both attached to a polymer (e.g., a hydrophobic polymer or a polymer containing a hydrophilic and a hydrophobic portion). In some embodiments, the counterion is attached to a polymer (e.g., a hydrophobic polymer or a polymer containing a hydrophilic and a hydrophobic portion), and the therapeutic peptide or protein is not attached to a polymer (e.g., the therapeutic peptide or protein is embedded in the particle). In some embodiments, neither the therapeutic peptide or protein nor counterion is attached to a polymer. The therapeutic peptide or protein and/or counterion can also be attached to other moieties. For example, the therapeutic peptide or protein can be attached to the counterion or to a hydrophilic polymer such as PEG.

In addition to a hydrophobic moiety such as a hydrophobic polymer or lipid (e.g., hydrophobic polymer), a polymer containing a hydrophilic portion and a hydrophobic portion, a therapeutic peptide or protein, and a counterion, the particles described herein may include one or more additional components such as an additional therapeutic peptide or protein or an additional counterion. A particle described herein may also include a compound having at least one acidic moiety, such as a carboxylic acid group. The compound may be a small molecule or a polymer having at least one acidic moiety. In some embodiments, the compound is a polymer such as PLGA.

In some embodiments, the particle is configured such that when administered to a subject there is preferential release of the therapeutic peptide or protein in a preselected compartment. The preselected compartment can be a target site, location, tissue type, cell type, e.g., a disease specific cell type, e.g., a cancer cell, or subcellular compartment, e.g., the cytosol. In an embodiment, a particle provides preferential release in a tumor, as opposed to other compartments, e.g., non-tumor compartments, e.g., the peripheral blood. In embodiments, where the therapeutic peptide or protein is attached to a polymer or a counterion, the therapeutic peptide or protein is released (e.g., through reductive cleavage of a linker) to a greater degree in a tumor than in non-tumor compartments, e.g., the peripheral blood, of a subject. In some embodiments, the particle is configured such that when administered to a subject, it delivers more therapeutic peptide or protein to a compartment of the subject, e.g., a tumor, than if the therapeutic peptide or protein were administered free.

In some embodiments, the particle is associated with an excipient, e.g., a carbohydrate component, or a stabilizer or lyoprotectant, e.g., a carbohydrate component, stabilizer or lyoprotectant described herein. While not wishing to be bound be theory the carbohydrate component may act as a stabilizer or lyoprotectant. In some embodiments, the carbohydrate component, stabilizer or lyoprotectant, comprises one or more carbohydrates (e.g., one or more carbohydrates described herein, such as, e.g., sucrose, cyclodextrin or a derivative of cyclodextrin (e.g. 2-hydroxypropyl-β-cyclodextrin, sometimes referred to herein as HP-β-CD)), salt, PEG, PVP or crown ether. In some embodiments, the carbohydrate component, stabilizer or lyoprotectant comprises two or more carbohydrates, e.g., two or more carbohydrates described herein. In one embodiment, the carbohydrate component, stabilizer or lyoprotectant includes a cyclic carbohydrate (e.g., cyclodextrin or a derivative of cyclodextrin, e.g., an α-, β-, or γ-, cyclodextrin (e.g. 2-hydroxypropyl-β-cyclodextrin)) and a non-cyclic carbohydrate. Exemplary non-cyclic oligosaccharides include those of less than 10, 8, 6 or 4 monosaccharide subunits (e.g., a monosaccharide or a disaccharide (e.g., sucrose, trehalose, lactose, maltose) or combinations thereof).

In an embodiment the carbohydrate component, stabilizer or lyoprotectant comprises a first and a second component, e.g., a cyclic carbohydrate and a non-cyclic carbohydrate, e.g., a mono-, di, or tetra saccharide.

In one embodiment, the weight ratio of cyclic carbohydrate to non-cyclic carbohydrate associated with the particle is a weight ratio described herein, e.g., 0.5:1.5 to 1.5:0.5.

In an embodiment the carbohydrate component, stabilizer or lyoprotectant comprises a first and a second component (designated here as A and B) as follows:
(A) comprises a cyclic carbohydrate and (B) comprises a disaccharide;
(A) comprises more than one cyclic carbohydrate, e.g., a β-cyclodextrin (sometimes referred to herein as β-CD) or a β-CD derivative, e.g., HP-β-CD, and
(B) comprises a disaccharide;
(A) comprises a cyclic carbohydrate, e.g., a β-CD or a β-CD derivative, e.g., HP-β-CD, and (B) comprises more than one disaccharide;
(A) comprises more than one cyclic carbohydrate, and (B) comprises more than one disaccharide;
(A) comprises a cyclodextrin, e.g., a β-CD or a β-CD derivative, e.g., HP-β-CD, and (B) comprises a disaccharide;
(A) comprises a β-cyclodextrin, e.g a β-CD derivative, e.g., HP-β-CD, and (B) comprises a disaccharide;
(A) comprises a β-cyclodextrin, e.g., a β-CD derivative, e.g., HP-β-CD, and (B) comprises sucrose;
(A) comprises a β-CD derivative, e.g., HP-β-CD, and (B) comprises sucrose;
(A) comprises a β-cyclodextrin, e.g., a β-CD derivative, e.g., HP-β-CD, and (B) comprises trehalose;

(A) comprises a β-cyclodextrin, e.g., a β-CD derivative, e.g., HP-β-CD, and (B) comprises sucrose and trehalose.

(A) comprises HP-β-CD, and (B) comprises sucrose and trehalose.

In an embodiment components A and B are present in the following ratio: 0.5:1.5 to 1.5:0.5. In an embodiment, components A and B are present in the following ratio: 3-1:0.4-2; 3-1:0.4-2.5; 3-1:0.4-2; 3-1:0.5-1.5; 3-1:0.5-1; 3-1: 1; 3-1:0.6-0.9; and 3:1:0.7. In an embodiment, components A and B are present in the following ratio: 2-1:0.4-2; 3-1:0.4-2.5; 2-1:0.4-2; 2-1:0.5-1.5; 2-1:0.5-1; 2-1:1; 2-1:0.6-0.9; and 2:1:0.7. In an embodiment components A and B are present in the following ratio: 2-1.5:0.4-2; 2-1.5:0.4-2.5; 2-1.5:0.4-2; 2-1.5:0.5-1.5; 2-1.5:0.5-1; 2-1.5:1; 2-1.5:0.6-0.9; 2:1.5:0.7. In an embodiment components A and B are present in the following ratio: 2.5-1.5:0.5-1.5; 2.2-1.6:0.7-1.3; 2.0-1.7:0.8-1.2; 1.8:1; 1.85:1 and 1.9:1.

In an embodiment component A comprises a cyclodextin, e.g., a β-cyclodextrin, e.g., a O-CD derivative, e.g., HP-β-CD, and (B) comprises sucrose, and they are present in the following ratio: 2.5-1.5:0.5-1.5; 2.2-1.6:0.7-1.3; 2.0-1.7:0.8-1.2; 1.8:1; 1.85:1 and 1.9:1.

In some embodiments, the particle includes a plurality of hydrophobic moieties. For example, the particle can include a hydrophobic polymer such as PLGA and another hydrophobic moiety such as chitosan, poly(vinyl alcohol), or a poloxamer.

In some embodiments, the particle includes a pH dampening molecule, for example, a compound that can act as a buffer. Exemplary pH dampening molecules include base salts (e.g., calcium carbonate, magnesium hydroxide and zinc carbonate) serve to buffer the system and proton sponges (e.g., amine groups), which can also help buffer the system.

A particle can also include a counterion, e.g., to counter a charge on the therapeutic peptide or protein. For example, if therapeutic peptide or protein-conjugate is positively charged exemplary counterions include acetic acid, adamantoic acid, alpha keto glutaric acid, D- or L-aspartic acid, benzensulfonic acid, benzoic acid, 10-camphorsulfunic acid, citric acid, 1,2-ethanedisulfonic acid, fumaric acid, D-gluconic acid, D-glucuronic acid, glucaric acid, D- or L-glutamic acid, glutaric acid, glycolic acid, hippuric acid, hydrobromic acid, hydrochloric acid, 1-hydroxyl-2-napthoic acid, lactobioinic acid, maleic acid, L-malic acid, mandelic acid, methanesulfonic acid, mucic acid, 1,5 napthalenedisulfonic acid tetrahydrate, 2-napthalenesulfonic acid, nitric acid, oleic acid, pamoic acid, phosphoric acid, p-toluenesulfonic acid hydrate, D-saccharid acid monopotassium salt, salicyclic acid, stearic acid, succinic acid, sulfuric acid, tannic acid, D- or L-tartaric acid. If the therapeutic peptide-conjugate is negatively charged, exemplary counterions include N-methyl D-glucamine, choline, arginine, lysine, procaine, tromethamine (TRIS), spermine, N-methyl-morpholine, glucosamine, N,N-bis 2-hydroxyethyl glycine, diazabicycloundecene, creatine, arginine ethyl ester, amantadine, rimantadine, ornithine, taurine, and citrulline.

In some embodiments, the particle is a nanoparticle. In some embodiments, the nanoparticle has a diameter of less than or equal to about 220 nm (e.g., less than or equal to about 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm or 50 nm). In an embodiment, the nanoparticle has a diameter of at least 10 nm (e.g., at least about 20 nm).

A particle described herein may also include a targeting agent or a lipid (e.g., on the surface of the particle).

A composition of a plurality of particles described herein may have an average diameter of about 50 nm to about 500 nm (e.g., from about 50 nm to about 200 nm). A composition of a plurality of particles particle may have a median particle size (Dᵥ50 (particle size below which 50% of the volume of particles exists) of about 50 nm to about 500 nm (e.g., about 75 nm to about 220 nm)) is from about 50 nm to about 220 nm (e.g., from about 75 nm to about 200 nm). A composition of a plurality of particles particle may have a Dᵥ90 (particle size below which 90% of the volume of particles exists) of about 50 nm to about 500 nm (e.g., about 75 nm to about 220 nm). In some embodiments, a composition of a plurality of particles has a Dv90 of less than about 150 nm. A composition of a plurality of particles may have a particle PDI of less than 0.5, less than 0.4, less than 0.3, less than 0.2, or less than 0.1.

A particle described herein may have a surface zeta potential ranging from about −20 mV to about 50 mV, when measured in water. Zeta potential is a measurement of surface potential of a particle. In some embodiments, a particle may have a surface zeta potential, when measured in water, ranging between about −20 mV to about 20 mV, about −10 mV to about 10 mV, or neutral.

In an embodiment, a particle, or a composition comprising a plurality of particles, described herein may, when stored at 25° C.±2° C./60% relative humidity ±5% relative humidity in an open, or closed, container, for 20, 30, 40, 50 or 60 days, retains at least 30, 40, 50, 60, 70, 80, 90, or 95% of its activity, e.g., as determined in an in vivo model system.

In an embodiment a particle is stable in non-polar organic solvent (e.g., any of hexane, chloroform, or dichloromethane). By way of example, the particle does not substantially invert, e.g., if present, an outer layer does not internalize, or a substantial amount of surface components do internalize, relative to their configuration in aqueous solvent. In embodiments the distribution of components is substantially the same in a non-polar organic solvent and in an aqueous solvent.

In an embodiment a particle lacks at least one component of a micelle, e.g., it lacks a core which is substantially free of hydrophilic components.

In an embodiment the core of the particle comprises a substantial amount of a hydrophilic component.

In an embodiment the core of the particle comprises a substantial amount e.g., at least 10, 20, 30, 40, 50, 60 or 70% (by weight or number) of the therapeutic peptide.

In an embodiment the core of the particle comprises a substantial amount e.g., at least 10, 20, 30, 40, 50, 60 or 70% (by weight or number) of the counterion, e.g., polycationic moiety, of the particle.

A particle described herein may include a small amount of a residual solvent, e.g., a solvent used in preparing the particles such as acetone, tert-butylmethyl ether, benzyl alcohol, dioxane, heptane, dichloromethane, dimethylformamide, dimethylsulfoxide, ethyl acetate, acetonitrile, tetrahydrofuran, ethanol, methanol, isopropyl alcohol, methyl ethyl ketone, butyl acetate, or propyl acetate (e.g., isopropylacetate). In some embodiments, the particle may include less than 5000 ppm of a solvent (e.g., less than 4500 ppm, less than 4000 ppm, less than 3500 ppm, less than 3000 ppm, less than 2500 ppm, less than 2000 ppm, less than 1500 ppm, less than 1000 ppm, less than 500 ppm, less than 250 ppm, less than 100 ppm, less than 50 ppm, less than 25 ppm, less than 10 ppm, less than 5 ppm, less than 2 ppm, or less than 1 ppm).

In some embodiments, the particle is substantially free of a class II or class III solvent as defined by the United States Department of Health and Human Services Food and Drug Administration “Q3c-Tables and List.” In some embodiments, the particle comprises less than 5000 ppm of acetone. In some embodiments, the particle comprises less than 5000 ppm of tert-butylmethyl ether. In some embodiments, the particle comprises less than 5000 ppm of heptane. In some embodiments, the particle comprises less than 600 ppm of dichloromethane. In some embodiments, the particle comprises less than 880 ppm of dimethylformamide. In some embodiments, the particle comprises less than 5000 ppm of ethyl acetate. In some embodiments, the particle comprises less than 410 ppm of acetonitrile. In some embodiments, the particle comprises less than 720 ppm of tetrahydrofuran. In some embodiments, the particle comprises less than 5000 ppm of ethanol. In some embodiments, the particle comprises less than 3000 ppm of methanol. In some embodiments, the particle comprises less than 5000 ppm of isopropyl alcohol. In some embodiments, the particle comprises less than 5000 ppm of methyl ethyl ketone. In some embodiments, the particle comprises less than 5000 ppm of butyl acetate. In some embodiments, the particle comprises less than 5000 ppm of propyl acetate.

A particle described herein may include varying amounts of a hydrophobic moiety such as a hydrophobic polymer, e.g., from about 20% to about 90% by weight of, or used as starting materials to make, the particle (e.g., from about 20% to about 80%, from about 25% to about 75%, or from about 30% to about 70%). A particle described herein may include varying amounts of a hydrophilic-hydrophobic polymer, e.g., up to about 50% by weight (e.g., from about 4 to any of about 50%, about 5%, about 8%, about 10%, about 15%, about 20%, about 23%, about 25%, about 30%, about 35%, about 40%, about 45% or about 50% by weight). For example, the percent by weight of the hydrophilic-hydrophobic polymer of the particle is from about 3% to 30%, from about 5% to 25% or from about 8% to 23%.

A particle described herein may include varying amounts of a counterion, e.g., from about 0.1% to about 60% by weight of, or used as starting materials to make, the particle (e.g., from about 1% to about 60%, from about 2% to about 20%, from about 3% to about 30%, from about 5% to about 40%, from about or from about 10% to about 30%).

A particle described herein may include varying amounts of therapeutic peptide, e.g., from about 0.1% to about 50% by weight of, or used as starting materials to make, the particle (e.g., from about 1% to about 50%, from about 0.5% to about 20%, from about 2% to about 20%, from about or from about 5% to about 15%).

When the particle includes a surfactant, the particle may include varying amounts of the surfactant, e.g., up to about 40% by weight of, or used as starting materials to make, the particle, or from about 15% to about 35% or from about 3% to about 10%. In some embodiments, the surfactant is PVA. In some embodiments, the particle may include about 2% to about 5% of PVA (e.g., about 4%) and from about 0.1% to about 3% cationic PVA (e.g., about 1%).

A particle described herein may be substantially free of a targeting agent (e.g., of a targeting agent covalently linked to a component in the particle e.g., a targeting agent able to bind to or otherwise associate with a target biological entity, e.g., a membrane component, a cell surface receptor, prostate specific membrane antigen, or the like. A particle described herein may be substantially free of a targeting agent selected from nucleic acid aptamers, growth factors, hormones, cytokines, interleukins, antibodies, integrins, fibronectin receptors, p-glycoprotein receptors, peptides and cell binding sequences. In some embodiments, no polymer within the particle is conjugated to a targeting moiety. A particle described herein may be free of moieties added for the purpose of selectively targeting the particle to a site in a subject, e.g., by the use of a moiety on the particle having a high and specific affinity for a target in the subject.

In some embodiments the particle is free of a lipid, e.g., free of a phospholipid. A particle described herein may be substantially free of an amphiphilic layer that reduces water penetration into the nanoparticle. A particle described herein may comprise less than 5 or 10% (e.g., as determined as w/w, v/v) of a lipid, e.g., a phospholipid. A particle described herein may be substantially free of a lipid layer, e.g., a phospholipid layer, e.g., that reduces water penetration into the nanoparticle. A particle described herein may be substantially free of lipid, e.g., is substantially free of phospholipid.

A particle described herein may be substantially free of a radiopharmaceutical agent, e.g., a radiotherapeutic agent, radiodiagnostic agent, prophylactic agent, or other radioisotope. A particle described herein may be substantially free of an immunomodulatory agent, e.g., an immunostimulatory agent or immunosuppressive agent. A particle described herein may be substantially free of a vaccine or immunogen, e.g., a peptide, sugar, lipid-based immunogen, B cell antigen or T cell antigen.

A particle described herein may be substantially free of a water-soluble hydrophobic polymer such as PLGA, e.g., PLGA having a molecular weight of less than about 1 kDa (e.g., less than about 500 Da).

Exemplary Particles

An exemplary particle includes a particle comprising:

a) a plurality of hydrophobic polymers;

b) a plurality of hydrophilic-hydrophobic polymers; and

c) a plurality of therapeutic peptides or proteins, wherein at least a portion of the plurality of therapeutic peptides or proteins are covalently attached to either of a hydrophobic polymer of a) or the hydrophilic-hydrophobic polymer b).

Another exemplary particle includes a particle comprising:

a) a plurality of therapeutic peptide or protein-polymer conjugates, comprising a therapeutic peptide or protein attached to a hydrophobic polymer; and

b) a plurality of hydrophilic-hydrophobic polymers.

Another exemplary particle includes a particle comprising:

a) optionally a plurality of hydrophobic polymers; and

b) a plurality of therapeutic peptide or protein-hydrophilic-hydrophobic polymer conjugate, comprising a therapeutic peptide or protein attached to the hydrophilic-hydrophobic polymer.

Another exemplary particle includes a particle comprising:

a) optionally, a plurality of hydrophobic polymers;

b) a plurality of hydrophilic-hydrophobic polymer-conjugates, wherein the hydrophilic-hydrophobic polymer conjugate comprises a hydrophilic-hydrophobic polymer attached to a charged peptide; and

c) a plurality of charged therapeutic peptides or proteins, wherein the charge of the therapeutic peptide or protein is opposite the charge of the peptide conjugated to the hydrophilic-hydrophobic polymer, and wherein the charged therapeutic peptide or protein forms a non-covalent bond (e.g., an ionic bond) with the charged peptide of the hydrophilic-hydrophobic polymer-conjugate.

Methods of Making Particles and Compositions

A particle described herein may be prepared using any method known in the art for preparing particles, e.g., nanoparticles. Exemplary methods include spray drying, emulsion (e.g., emulsion-solvent evaporation or double emulsion), precipitation (e.g., nanoprecipitation) and phase inversion.

In one embodiment, a particle described herein can be prepared by precipitation (e.g., nanoprecipitation). This method involves dissolving the components of the particle (i.e., one or more polymers, an optional additional component or components, and an agent), individually or combined, in one or more solvents to form one or more solutions. For example, a first solution containing one or more of the components may be poured into a second solution containing one or more of the components (at a suitable rate or speed). The solutions may be combined, for example, using a syringe pump, a MicroMixer, or any device that allows for vigorous, controlled mixing. In some cases, nanoparticles can be formed as the first solution contacts the second solution, e.g., precipitation of the polymer upon contact causes the polymer to form nanoparticles. The control of such particle formation can be readily optimized.

In one set of embodiments, the particles are formed by providing one or more solutions containing one or more polymers and additional components, and contacting the solutions with certain solvents to produce the particle. In a non-limiting example, a hydrophobic polymer (e.g., PLGA), is conjugated to a therapeutic peptide or protein to form a conjugate. This therapeutic peptide or protein-polymer conjugate, a polymer containing a hydrophilic portion and a hydrophobic portion (e.g., PEG-PLGA), and optionally a third polymer (e.g., a biodegradable polymer, e.g., PLGA) are dissolved in a partially water miscible organic solvent (e.g., acetone). This solution is added to an aqueous solution containing a surfactant, forming the desired particles. These two solutions may be individually sterile filtered prior to mixing/precipitation.

The formed nanoparticles can be exposed to further processing techniques to remove the solvents or purify the nanoparticles (e.g., dialysis). For purposes of the aforementioned process, water miscible solvents include acetone, ethanol, methanol, and isopropyl alcohol; and partially water miscible organic solvents include acetonitrile, tetrahydrofuran, ethyl acetate, isopropyl alcohol, isopropyl acetate or dimethylformamide.

Another method that can be used to generate a particle described herein is a process termed “flash nanoprecipitation” as described by Johnson, B. K., et al, AlChE Journal (2003) 49:2264-2282 and U.S. 2004/0091546, each of which is incorporated herein by reference in its entirety. This process is capable of producing controlled size, polymer-stabilized and protected nanoparticles of hydrophobic organics at high loadings and yields. The flash nanoprecipitation technique is based on amphiphilic diblock copolymer arrested nucleation and growth of hydrophobic organics. Amphiphilic diblock copolymers dissolved in a suitable solvent can form micelles when the solvent quality for one block is decreased. In order to achieve such a solvent quality change, a tangential flow mixing cell (vortex mixer) is used. The vortex mixer consists of a confined volume chamber where one jet stream containing the diblock copolymer and active agent dissolved in a water-miscible solvent is mixed at high velocity with another jet stream containing water, an anti-solvent for the active agent and the hydrophobic block of the copolymer. The fast mixing and high energy dissipation involved in this process provide timescales that are shorter than the timescale for nucleation and growth of particles, which leads to the formation of nanoparticles with active agent loading contents and size distributions not provided by other technologies. When forming the nanoparticles via flash nanoprecipitation, mixing occurs fast enough to allow high supersaturation levels of all components to be reached prior to the onset of aggregation. Therefore, the active agent(s) and polymers precipitate simultaneously, and overcome the limitations of low active agent incorporations and aggregation found with the widely used techniques based on slow solvent exchange (e.g., dialysis). The flash nanoprecipitation process is insensitive to the chemical specificity of the components, making it a universal nanoparticle formation technique.

A particle described herein may also be prepared using a mixer technology, such as a static mixer or a micro-mixer (e.g., a split-recombine micro-mixer, a slit-interdigital micro-mixer, a star laminator interdigital micro-mixer, a superfocus interdigital micro-mixer, a liquid-liquid micro-mixer, or an impinging jet micro-mixer).

A split-recombine micromixer uses a mixing principle involving dividing the streams, folding/guiding over each other and recombining them per each mixing step, consisting of 8 to 12 such steps. Mixing finally occurs via diffusion within milliseconds, exclusive of residence time for the multi-step flow passage. Additionally, at higher-flow rates, turbulences add to this mixing effect, improving the total mixing quality further.

A slit interdigital micromixer combines the regular flow pattern created by multi-lamination with geometric focusing, which speeds up liquid mixing. Due to this double-step mixing, a slit mixer is amenable to a wide variety of processes.

A particle described herein may also be prepared using Microfluidics Reaction Technology (MRT). At the core of MRT is a continuous, impinging jet microreactor scalable to at least 50 lit/min. In the reactor, high-velocity liquid reactants are forced to interact inside a microliter scale volume. The reactants mix at the nanometer level as they are exposed to high shear stresses and turbulence. MRT provides precise control of the feed rate and the mixing location of the reactants. This ensures control of the nucleation and growth processes, resulting in uniform crystal growth and stabilization rates.

A particle described herein may also be prepared by emulsion. An exemplary emulsification method is disclosed in U.S. Pat. No. 5,407,609, which is incorporated herein by reference. This method involves dissolving or otherwise dispersing agents, liquids or solids, in a solvent containing dissolved wall-forming materials, dispersing the agent/polymer-solvent mixture into a processing medium to form an emulsion and transferring all of the emulsion immediately to a large volume of processing medium or other suitable extraction medium, to immediately extract the solvent from the microdroplets in the emulsion to form a microencapsulated product, such as microcapsules or microspheres. The most common method used for preparing polymer delivery vehicle formulations is the solvent emulsification-evaporation method. This method involves dissolving the polymer and drug in an organic solvent that is completely immiscible with water (for example, dichloromethane). The organic mixture is added to water containing a stabilizer, most often poly(vinyl alcohol) (PVA) and then typically sonicated.

After the particles are prepared, they may be fractionated by filtering, sieving, extrusion, or ultracentrifugation to recover particles within a specific size range. One sizing method involves extruding an aqueous suspension of the particles through a series of polycarbonate membranes having a selected uniform pore size; the pore size of the membrane will correspond roughly with the largest size of particles produced by extrusion through that membrane. See, e.g., U.S. Pat. No. 4,737,323, incorporated herein by reference. Another method is serial ultracentrifugation at defined speeds (e.g., 8,000, 10,000, 12,000, 15,000, 20,000, 22,000, and 25,000 rpm) to isolate fractions of defined sizes. Another method is tangential flow filtration, wherein a solution containing the particles is pumped tangentially along the surface of a membrane. An applied pressure serves to force a portion of the fluid through the membrane to the filtrate side. Particles that are too large to pass through the membrane pores are retained on the upstream side. The retained components do not build up at the surface of the membrane as in normal flow filtration, but instead are swept along by the tangential flow. Tangential flow filtration may thus be used to remove excess surfactant present in the aqueous solution or to concentrate the solution via diafiltration.

After purification of the particles, they may be sterile filtered (e.g., using a 0.22 micron filter) while in solution.

In certain embodiments, the particles are prepared to be substantially homogeneous in size within a selected size range. The particles are preferably in the range from 30 nm to 300 nm in their greatest diameter, (e.g., from about 30 nm to about 250 nm). The particles may be analyzed by techniques known in the art such as dynamic light scattering and/or electron microscopy, (e.g., transmission electron microscopy or scanning electron microscopy) to determine the size of the particles. The particles may also be tested for agent loading and/or the presence or absence of impurities.

Lyophilization

A particle described herein may be prepared for dry storage via lyophilization, commonly known as freeze-drying. Lyophilization is a process which extracts water from a solution to form a granular solid or powder. The process is carried out by freezing the solution and subsequently extracting any water or moisture by sublimation under vacuum. Advantages of lyophilization include maintenance of substance quality and minimization of therapeutic compound degradation. Lyophilization may be particularly useful for developing pharmaceutical drug products that are reconstituted and administered to a patient by injection, for example parenteral drug products. Alternatively, lyophilization is useful for developing oral drug products, especially fast melts or flash dissolve formulations.

Lyophilization may take place in the presence of a lyoprotectant, e.g., a lyoprotectant described herein. In some embodiments, the lyoprotectant is a carbohydrate (e.g., a carbohydrate described herein, such as, e.g., sucrose, cyclodextrin or a derivative of cyclodextrin (e.g. 2-hydroxypropyl-β-cyclodextrin)), salt, PEG, PVP or crown ether.

Therapeutic Peptide or Protein-Polymer Conjugates

A therapeutic peptide or protein-polymer conjugate described herein includes a polymer (e.g., a hydrophobic polymer or a hydrophilic-hydrophobic polymer) and a therapeutic peptide or protein. A therapeutic peptide or protein described herein may be attached to a polymer described herein, e.g., directly or through a linker A therapeutic peptide or protein may be attached to a hydrophobic polymer (e.g., PLGA), or a polymer having a hydrophobic portion and a hydrophilic portion (e.g., PEG-PLGA). A therapeutic peptide or protein may be attached to a terminal end of a polymer, to both terminal ends of a polymer, or to a point along a polymer chain. In some embodiments, multiple therapeutic peptides or proteins may be attached to points along a polymer chain, or multiple therapeutic peptides or proteins may be attached to a terminal end of a polymer via a multifunctional linker

Polymers

A wide variety of polymers and methods for forming therapeutic peptide or protein-polymer conjugates and particles therefrom are known in the art of therapeutic peptide delivery. Any polymer may be used in accordance with the present invention. Polymers may be natural or unnatural (synthetic) polymers. Polymers may be homopolymers or copolymers containing two or more monomers. Polymers may be linear or branched.

If more than one type of repeat unit is present within the polymer, then the polymer is said to be a “copolymer.” It is to be understood that in any embodiment employing a polymer, the polymer being employed may be a copolymer. The repeat units forming the copolymer may be arranged in any fashion. For example, the repeat units may be arranged in a random order, in an alternating order, or as a “block” copolymer, i.e., containing one or more regions each containing a first repeat unit (e.g., a first block), and one or more regions each containing a second repeat unit (e.g., a second block), etc. Block copolymers may have two (a diblock copolymer), three (a triblock copolymer), or more numbers of distinct blocks. In terms of sequence, copolymers may be random, block, or contain a combination of random and block sequences.

Hydrophobic Moieties

Hydrophobic Polymers

A particle described herein may include a hydrophobic polymer. The hydrophobic polymer may be attached to a therapeutic peptide or protein and/or counterion to form a conjugate (e.g., a therapeutic peptide/protein-hydrophobic polymer conjugate or counterion-hydrophobic polymer conjugate).

In some embodiments, the hydrophobic polymer is not attached to another moiety. A particle can include a plurality of hydrophobic polymers, for example where some are attached to another moiety such as a therapeutic peptide and/or counterion, and some are free.

Exemplary hydrophobic polymers include the following: acrylates including methyl acrylate, ethyl acrylate, propyl acrylate, n-butyl acrylate (BA), isobutyl acrylate, 2-ethyl acrylate, and t-butyl acrylate; methacrylates including ethyl methacrylate, n-butyl methacrylate, and isobutyl methacrylate; acrylonitriles; methacrylonitrile; vinyls including vinyl acetate, vinylversatate, vinylpropionate, vinylformamide, vinylacetamide, vinylpyridines, and vinylimidazole; aminoalkyls including aminoalkylacrylates, aminoalkylmethacrylates, and aminoalkyl(meth)acrylamides; styrenes; cellulose acetate phthalate; cellulose acetate succinate; hydroxypropylmethylcellulose phthalate; poly(D,L-lactide); poly(D,L-lactide-co-glycolide); poly(glycolide); poly(hydroxybutyrate); poly(alkylcarbonate); poly(orthoesters); polyesters; poly(hydroxyvaleric acid); polydioxanone; poly(ethylene terephthalate); poly(malic acid); poly(tartronic acid); polyanhydrides; polyphosphazenes; poly(amino acids) and their copolymers (see generally, Svenson, S (ed.)., Polymeric Drug Delivery: Volume I: Particulate Drug Carriers. 2006; ACS Symposium Series; Amiji, M. M (ed.)., Nanotechnology for Cancer Therapy. 2007; Taylor & Francis Group, LLP; Nair et al. Prog. Polym. Sci. (2007) 32: 762-798); hydrophobic peptide-based polymers and copolymers based on poly(L-amino acids) (Lavas anifar, A., et al., Advanced Drug Delivery Reviews (2002) 54:169-190); poly(ethylene-vinyl acetate) (“EVA”) copolymers; silicone rubber; polyethylene; polypropylene; polydienes (polybutadiene, polyisoprene and hydrogenated forms of these polymers); maleic anhydride copolymers of vinyl methylether and other vinyl ethers; polyamides (nylon 6,6); polyurethane; poly(ester urethanes); poly(ether urethanes); and poly(ester-urea).

Hydrophobic polymers useful in preparing the polymer-agent conjugates or particles described herein also include biodegradable polymers. Examples of biodegradable polymers include polylactides, polyglycolides, caprolactone-based polymers, poly(caprolactone), polydioxanone, polyanhydrides, polyamines, polyesteramides, polyorthoesters, polydioxanones, polyacetals, polyketals, polycarbonates, polyphosphoesters, polyesters, polybutylene terephthalate, polyorthocarbonates, polyphosphazenes, succinates, poly(malic acid), poly(amino acids), poly(vinylpyrrolidone), polyethylene glycol, polyhydroxycellulose, polysaccharides, chitin, chitosan and hyaluronic acid, and copolymers, terpolymers and mixtures thereof. Biodegradable polymers also include copolymers, including caprolactone-based polymers, polycaprolactones and copolymers that include polybutylene terephthalate.

In some embodiments, the polymer is a polyester synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxy hexanoic acid, γ-butyrolactone, γ-hydroxy butyric acid, δ-valerolactone, δ-hydroxy valeric acid, hydroxybutyric acids, and malic acid.

A copolymer may also be used in a polymer-agent conjugate or particle described herein. In some embodiments, a polymer may be PLGA, which is a biodegradable random copolymer of lactic acid and glycolic acid. A PLGA polymer may have varying ratios of lactic acid:glycolic acid, e.g., ranging from about 0.1:99.9 to about 99.9:0.1 (e.g., from about 75:25 to about 25:75, from about 60:40 to 40:60, or about 55:45 to 45:55). In some embodiments, e.g., in PLGA, the ratio of lactic acid monomers to glycolic acid monomers is 50:50, 60:40 or 75:25.

In particular embodiments, by optimizing the ratio of lactic acid to glycolic acid monomers in the PLGA polymer of the polymer-agent conjugate or particle, parameters such as water uptake, agent release (e.g., “controlled release”) and polymer degradation kinetics may be optimized. Furthermore, tuning the ratio will also affect the hydrophobicity of the copolymer, which may in turn affect drug loading.

In certain embodiments wherein the biodegradable polymer also has a therapeutic peptide, protein or other material such as a counterion attached to it, the biodegradation rate of such polymer may be characterized by a release rate of such materials. In such circumstances, the biodegradation rate may depend on not only the chemical identity and physical characteristics of the polymer, but also on the identity of material(s) attached thereto. Degradation of the subject compositions includes not only the cleavage of intramolecular bonds, e.g., by oxidation and/or hydrolysis, but also the disruption of intermolecular bonds, such as dissociation of host/guest complexes by competitive complex formation with foreign inclusion hosts. In some embodiments, the release can be affected by an additional component in the particle, e.g., a compound having at least one acidic moiety (e.g., free-acid PLGA).

In certain embodiments, particles comprising one or more polymers, such as a hydrophobic polymer, biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 4 and 8 having a temperature of between 25° C. and 37° C. In other embodiments, the polymer degrades in a period of between about one hour and several weeks, depending on the desired application.

When polymers are used for delivery of therapeutic peptides in vivo, it is important that the polymers themselves be nontoxic and that they degrade into non-toxic degradation products as the polymer is eroded by the body fluids. Many synthetic biodegradable polymers, however, yield oligomers and monomers upon erosion in vivo that adversely interact with the surrounding tissue (D. F. Williams, J. Mater. Sci. 1233 (1982)). To minimize the toxicity of the intact polymer carrier and its degradation products, polymers have been designed based on naturally occurring metabolites. Exemplary polymers include polyesters derived from lactic and/or glycolic acid and polyamides derived from amino acids.

A number of biodegradable polymers are known and used for controlled release of pharmaceuticals. Such polymers are described in, for example, U.S. Pat. Nos. 4,291,013; 4,347,234; 4,525,495; 4,570,629; 4,572,832; 4,587,268; 4,638,045; 4,675,381; 4,745,160; and 5,219,980; and PCT publication WO2006/014626, each of which is hereby incorporated by reference in its entirety.

A hydrophobic polymer described herein may have a variety of end groups. In some embodiments, the end group of the polymer is not further modified, e.g., when the end group is a carboxylic acid, a hydroxy group or an amino group. In some embodiments, the end group may be further modified. For example, a polymer with a hydroxyl end group may be derivatized with an acyl group to yield an acyl-capped polymer (e.g., an acetyl-capped polymer or a benzoyl capped polymer), an alkyl group to yield an alkoxy-capped polymer (e.g., a methoxy-capped polymer), or a benzyl group to yield a benzyl-capped polymer. The end group can also be further reacted with a functional group, for example to provide a linkage to another moiety such as a nucliec acid agent, a counterion, or an insoluble substrate. In some embodiments a particle comprises a functionalized hydrophobic polymer, e.g., a hydrophobic polymer, such as PLGA (e.g., 50:50 PLGA), functionalized with a moiety, e.g., N-(2-aminoethyl)maleimide, 2-(2-(pyridine-2-yl)disulfanyl)ethylamino, or a succinimidyl-N-methyl ester, that has not reacted with another moiety, e.g., a therapeutic peptide.

A hydrophobic polymer may have a weight average molecular weight ranging from about 1 kDa to about 70 kDa (e.g., from about 4 kDa to about 66 kDa, from about 2 kDa to about 12 kDa, from about 6 kDa to about 20 kDa, from about 5 kDa to about 15 kDa, from about 6 kDa to about 13 kDa, from about 7 kDa to about 11 kDa, from about 5 kDa to about 10 kDa, from about 7 kDa to about 10 kDa, from about 5 kDa to about 7 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

A hydrophobic polymer described herein may have a polymer polydispersity index (PDI) of less than or equal to about 2.5 (e.g., less than or equal to about 2.2, less than or equal to about 2.0, or less than or equal to about 1.5). In some embodiments, a hydrophobic polymer described herein may have a polymer PDI of about 1.0 to about 2.5, about 1.0 to about 2.0, about 1.0 to about 1.7, or from about 1.0 to about 1.6.

A particle described herein may include varying amounts of a hydrophobic polymer, e.g., from about 10% to about 90% by weight of the particle (e.g., from about 20% to about 80%, from about 25% to about 75%, or from about 30% to about 70%).

A hydrophobic polymer described herein may be commercially available, e.g., from a commercial supplier such as BASF, Boehringer Ingelheim, Durcet Corporation, Purac America and SurModics Pharmaceuticals. A polymer described herein may also be synthesized. Methods of synthesizing polymers are known in the art (see, for example, Polymer Synthesis: Theory and Practice Fundamentals, Methods, Experiments. D. Braun et al., 4th edition, Springer, Berlin, 2005). Such methods include, for example, polycondensation, radical polymerization, ionic polymerization (e.g., cationic or anionic polymerization), or ring-opening metathesis polymerization.

A commercially available or synthesized polymer sample may be further purified prior to formation of a polymer-agent conjugate or incorporation into a particle or composition described herein. In some embodiments, purification may reduce the polydispersity of the polymer sample. A polymer may be purified by precipitation from solution, or precipitation onto a solid such as Celite. A polymer may also be further purified by size exclusion chromatography (SEC).

Other Hydrophobic Moieties

Other suitable hydrophobic moieties for the particles described herein include lipids e.g., a phospholipid. Exemplary lipids include lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, and dilinoleoylphosphatidylcholine.

Other exemplary hydrophobic moieties include cholesterol and Vitamin E TPGS.

In an embodiment, the hydrophobic moiety is not a lipid (e.g., not a phospholipid) or does not comprise a lipid.

Hydrophobic-Hydrophilic Polymers

A particle described herein may include a polymer containing a hydrophilic portion and a hydrophobic portion, e.g., a hydrophobic-hydrophilic polymer. The hydrophobic-hydrophilic polymer may be attached to another moiety such as a therapeutic peptide or protein (e.g., through the hydrophilic or hydrophobic portion). In some embodiments, the hydrophobic-hydrophilic polymer is free (i.e., not attached to another moiety). A particle can include a plurality of hydrophobic-hydrophilic polymers, for example where some are attached to another moiety such as a therapeutic peptide, protein and/or counterion and some are free.

A polymer containing a hydrophilic portion and a hydrophobic portion may be a copolymer of a hydrophilic block coupled with a hydrophobic block. These copolymers may have a weight average molecular weight between about 5 kDa and about 30 kDa (e.g., from about 5 kDa to about 25 kDa, from about 10 kDa to about 22 kDa, from about 10 kDa to about 15 kDa, from about 12 kDa to about 22 kDa, from about 7 kDa to about 15 kDa, from about 15 kDa to about 19 kDa, or from about 11 kDa to about 13 kDa, e.g., about 9 kDa, about 10 kDa, about 11 kDa, about 12 kDa, about 13 kDa, about 14 kDa about 15 kDa, about 16 kDa, about 17 kDa, about 18 kDa or about 19 kDa). The polymer containing a hydrophilic portion and a hydrophobic portion may be attached to an agent.

Examples of suitable hydrophobic portions of the polymers include those described above. The hydrophobic portion of the copolymer may have a weight average molecular weight of from about 1 kDa to about 20 kDa (e.g., from about 8 kDa to about 15, kDa from about 1 kDa to about 18 kDa, 17 kDa, 16 kDa, 15 kDa, 14 kDa or 13 kDa, from about 2 kDa to about 12 kDa, from about 6 kDa to about 20 kDa, from about 5 kDa to about 18 kDa, from about 7 kDa to about 17 kDa, from about 8 kDa to about 13 kDa, from about 9 kDa to about 11 kDa, from about 10 kDa to about 14 kDa, from about 6 kDa to about 8 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa or about 17 kDa).

Examples of suitable hydrophilic portions of the polymers include the following: carboxylic acids including acrylic acid, methacrylic acid, itaconic acid, and maleic acid; polyoxyethylenes or polyethylene oxide (PEG); polyacrylamides (e.g. polyhydroxylpropylmethacrylamide), and copolymers thereof with dimethylaminoethylmethacrylate, diallyldimethylammonium chloride, vinylbenzylthrimethylammonium chloride, acrylic acid, methacrylic acid, 2-acrylamido-2-methylpropane sulfonic acid and styrene sulfonate, poly(vinylpyrrolidone), polyoxazoline, polysialic acid, starches and starch derivatives, dextran and dextran derivatives; polypeptides, such as polylysines, polyarginines, polyglutamic acids; polyhyaluronic acids, alginic acids, polylactides, polyethyleneimines, polyionenes, polyacrylic acids, and polyiminocarboxylates, gelatin, and unsaturated ethylenic mono or dicarboxylic acids. A listing of suitable hydrophilic polymers can be found in Handbook of Water-Soluble Gums and Resins, R. Davidson, McGraw-Hill (1980). The hydrophilic portion of the copolymer may have a weight average molecular weight of from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is PEG, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is PVA, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is polyoxazoline, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is polyvinylpyrrolidine, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is polyhydroxylpropylmethacrylamide, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa). In one embodiment, the hydrophilic portion is polysialic acid, and the weight average molecular weight is from about 1 kDa to about 21 kDa (e.g., from about 1 kDa to about 8 kDa, from about 1 kDa to about 3 kDa, e.g., about 2 kDa, or from about 2 kDa to about 6 kDa, e.g., about 3.5 kDa, or from about 4 kDa to about 6 kDa, e.g., about 5 kDa).

A polymer containing a hydrophilic portion and a hydrophobic portion may be a block copolymer, e.g., a diblock or triblock copolymer. In some embodiments, the polymer may be a diblock copolymer containing a hydrophilic block and a hydrophobic block. In some embodiments, the polymer may be a triblock copolymer containing a hydrophobic block, a hydrophilic block and another hydrophobic block. The two hydrophobic blocks may be the same hydrophobic polymer or different hydrophobic polymers. The block copolymers used herein may have varying ratios of the hydrophilic portion to the hydrophobic portion, e.g., ranging from 1:1 to 1:40 by weight (e.g., about 1:1 to about 1:10 by weight, about 1:1 to about 1:2 by weight, or about 1:3 to about 1:6 by weight).

A polymer containing a hydrophilic portion and a hydrophobic portion may have a variety of end groups. In some embodiments, the end group may be a hydroxy group or an alkoxy group (e.g., methoxy). In some embodiments, the end group of the polymer is not further modified. In some embodiments, the end group may be further modified. For example, the end group may be capped with an alkyl group, to yield an alkoxy-capped polymer (e.g., a methoxy-capped polymer), may be derivatized with a targeting agent (e.g., folate) or a dye (e.g., rhodamine), or may be reacted with a functional group.

A polymer containing a hydrophilic portion and a hydrophobic portion may include a linker between the two blocks of the copolymer. Such a linker may be an amide, ester, ether, amino, carbamate or carbonate linkage, for example.

A polymer containing a hydrophilic portion and a hydrophobic portion described herein may have a polymer polydispersity index (PDI) of less than or equal to about 2.5 (e.g., less than or equal to about 2.2, or less than or equal to about 2.0, or less than or equal to about 1.5). In some embodiments, the polymer PDI is from about 1.0 to about 2.5, e.g., from about 1.0 to about 2.0, from about 1.0 to about 1.8, from about 1.0 to about 1.7, or from about 1.0 to about 1.6.

A particle described herein may include varying amounts of a polymer containing a hydrophilic portion and a hydrophobic portion, e.g., up to about 50% by weight of the particle (e.g., from about 4 to about 50%, about 5%, about 10%, about 15%, about 20%, about 25%, about 30%, about 35%, about 40%, about 45% or about 50% by weight). For example, the percent by weight of the second polymer within the particle is from about 3% to 30%, from about 5% to 25% or from about 8% to 23%.

A polymer containing a hydrophilic portion and a hydrophobic portion described herein may be commercially available, or may be synthesized. Methods of synthesizing polymers are known in the art (see, for example, Polymer Synthesis: Theory and Practice Fundamentals, Methods, Experiments. D. Braun et al., 4th edition, Springer, Berlin, 2005). Such methods include, for example, polycondensation, radical polymerization, ionic polymerization (e.g., cationic or anionic polymerization), or ring-opening metathesis polymerization. A block copolymer may be prepared by synthesizing the two polymer units separately and then conjugating the two portions using established methods. For example, the blocks may be linked using a coupling agent such as EDC (1-ethyl-3-(3-dimethylaminopropyl) carbodiimide hydrochloride). Following conjugation, the two blocks may be linked via an amide, ester, ether, amino, carbamate or carbonate linkage.

A commercially available or synthesized polymer sample may be further purified prior to formation of a polymer-agent conjugate or incorporation into a particle or composition described herein. In some embodiments, purification may remove lower molecular weight polymers that may lead to unfilterable polymer samples. A polymer may be purified by precipitation from solution, or precipitation onto a solid such as Celite. A polymer may also be further purified by size exclusion chromatography (SEC).

Peptide-Polymer Conjugates

In some embodiments a polymer such as a hydrophilic-hydrophobic polymer is attached to a charged peptide. A charged therapeutic peptide or protein can then form a non-covalent bond with the charged peptide. Charged peptides can form conjugates with the same polymers as described above (e.g., hydrophobic and hydrophilic-hydrophobic polymers) using the same methods as described above.

Therapeutic Peptides

Therapeutic peptides can be delivered to a subject using a therapeutic peptide-polymer conjugate, particle or composition described. In some embodiments, the therapeutic peptide is a compound with pharmaceutical activity. In another embodiment, the therapeutic peptide is a clinically used or investigated drug. In another embodiment, the therapeutic peptide has been approved by the U.S. Food and Drug Administration for use in humans or other animals. In some embodiments the therapeutic peptide is a charged peptide (e.g., having a positive or negative charge).

Metabolic Disorders

The disclosed therapeutic peptide-polymer conjugates, particles and compositions may be useful in the prevention and treatment of metabolic disorders.

In some embodiments, the therapeutic peptide is a hormone. Examples of hormones include enkephalin, GLP-1 (e.g., GLP-1 (7-37), GLP-1 (7-36)), GLP-2, insulin, insulin-like growth factor-1, insulin-like growth factor-2, orexin A, orexin B, neuropeptide Y, growth hormone-releasing hormone, thryotropin-releasing hormone, cholecystokinin, melanocyte-stimulating hormone, corticotrophin-releasing factor, melanin concentrating hormone, galanin, bombesin, calcitonin gene related peptide, neurotensin, endorphin, dynorpin, and the C-peptide of proinsulin.

Preferably, the therapeutic peptide is an anti-diabetogenic peptide. An anti-diabetogenic peptide includes a peptide having one or more of the following activities: 1) ability to increase insulin secretion; 2) ability to increase insulin biosynthesis; 3) ability to decrease glucagon secretion; 4) ability to delay gastric emptying; 5) reduce hepatic gluconeogenesis; 6) improve insulin sensitivity; 7) improve glucose sensing by the beta cell; 8) enhance glucose disposal; 9) reduce insulin resistance; and 10) promote beta cell function or viability. Examples of anti-diabetogenic peptides include glucagon-like peptide-1 (GLP-1), insulin, insulin-like growth factor-1, insulin-like growth factor-2, exedin-4 and gastric inhibitory polypeptide and variants and derivatives thereof. Variants of some of the small peptides listed above are known. For example, know variants of GLP-1 include, for example, GLP-1 (7-36), GLP-1 (7-37), Gln⁹-GLP-1 (7-37), Thr¹⁶-Lys¹⁸-GLP-1 (7-37), Lys¹⁸-GLP-1 (7-37) and Gly⁸-GLP-1. Derivatives include, for example, acid addition salts, carboxylate salts, lower alkyl esters, and amides such as those described in PCT Publication WO 91/11457.

Exemplary therapeutic peptides include:
A-71378 (Abbott Laboratories) which is a six amino acid peptide (and variants and derivatives thereof) that can be used in the particles, conjugates and compositions described herein to treat metabolic disorders such as obesity;

PYY 3-36 (Amylin Pharmaceuticals) a thirty-four amino acid peptide (and variants and derivatives thereof) that can be used in the particles, conjugates and compositions described herein to treat metabolic disorders such as obesity;

AC-253 (Antam, Amylin Pharmaceuticals), and variants and derivatives thereof, which can be used in the particles, conjugates and compositions described herein to treat metabolic disorders such as diabetes (e.g., type 1 diabetes, type 2 diabetes, and/or gestational diabetes) and obesity;

albiglutide (GSK-716155, Syncria, GlaxoSmithKline), and variants and derivatives thereof, which can be used in the particles, conjugates and compositions described herein to treat metabolic disorders such as diabetes (e.g., type 1 diabetes, type 2 diabetes, gestational diabetes);

AKL-0707 (LAB GHRH, Akela Pharma), a 29 amino acid peptide, and variants and derivatives thereof, which can be used in the particles, conjugates and compositions described herein to treat metabolic disorders such as lipid metabolism disorder and malnutrition;

AOD-9604 (Metabolic Pharmaceuticals, Ltd.), a cyclic 16 amino acid peptide, and variants and derivatives thereof, which can be used in the particles, conjugates and compositions described herein to treat metabolic disorders such as obesity;

BAY-73-7977 (Bayer AG), and variants and derivatives thereof, which can be used in the particles, conjugates and compositions described herein to treat a metabolic disorder such as diabetes (e.g., type 1 diabetes, type 2 diabetes, and gestational diabetes);

BMS-686117 (Bristol-Myers Squibb), an eleven amino acid peptide, and variants and derivatives thereof, which can be used in the particles, conjugates and compositions described herein to treat metabolic disorders such as diabetes (e.g., type 1 diabetes, type 2 diabetes, and gestational diabetes);

BIM-44002 (Ipsen), a twenty-eight amino acid peptide, and variants and derivatives thereof, which can be used in the particles, conjugates and compositions described herein to treat metabolic disorders such as hypercalcemia;

CVX-096 (Pfizer-Covx), and variants and derivatives thereof, which can be used in the particles, conjugates and compositions described herein to treat a metabolic disorder such as diabetes (e.g., type 1 diabetes, type 2 diabetes, and gestational diabetes);

davalintide (AC-2307, Amylin Pharmaceuticals), a cyclic thirty amino acid peptide, and variants and derivatives thereof, which can be used in the particles, conjugates and compositions described herein to treat a metabolic disorder such as obesity;

AC-2993 (LY-2148568, Byetta™, Amylin Pharmaceuticals) a thirty-eight amino acid peptide, and variants and derivatives thereof, which can be used in the particles, conjugates and compositions described herein to treat a metabolic disorder such as diabetes (e.g., type 1 diabetes, type 2 diabetes, gestational diabetes) and obesity;

exsulin (INGAP peptide, Exsulin), a fifteen amino acid peptide, and variants and derivatives thereof, which can be used in the particles, conjugates and compositions described herein to treat a metabolic disorder such as diabetes (e.g., type 1 diabetes, type 2 diabetes, gestational diabetes);

glucagon (Glucogen™, Novo Nordisk), a twenty-nine amino acid peptide, and variants and derivatives thereof, which can be used in the particles, conjugates and compositions described herein to treat a metabolic disorder such as diabetes (e.g., type 1 diabetes, type 2 diabetes, gestational diabetes);

ISF402 (Dia-B Tech), a four amino acid peptide, and variants and derivatives thereof, which can be used in the particles, conjugates and compositions described herein to treat a metabolic disorder such as diabetes (e.g., type 1 diabetes, type 2 diabetes, gestational diabetes);

larazotide (AT-1001, SPD-550, Alba Therapeutics Corp), an eight amino acid peptide, and variants and derivatives thereof, which can be used in the particles, conjugates and compositions described herein to treat a metabolic disorder such as diabetes (e.g., type 1 diabetes, type 2 diabetes, gestational diabetes);

liraglutide (Victoza™, Novo Nordisk), a thirty-one amino acid peptide, and variants and derivatives thereof, which can be used in the particles, conjugates and compositions described herein to treat a metabolic disorder such as diabetes (e.g., type 1 diabetes, type 2 diabetes, gestational diabetes) and obesity;

lixisenatide (AVE-0010, ZP-10, Sanofi Aventis), a forty-four amino acid peptide, and variants and derivatives thereof, which can be used in the particles, conjugates and compositions described herein to treat a metabolic disorder such as diabetes (e.g., type 1 diabetes, type 2 diabetes, gestational diabetes);

LY-2189265 (Eli Lilly & Co.), and variants and derivatives thereof, which can be used in the particles, conjugates and compositions described herein to treat a metabolic disorder such as diabetes (e.g., type 1 diabetes, type 2 diabetes, gestational diabetes);

LY-548805 (Eli Lilly & Co.), and variants and derivatives thereof, which can be used in the particles, conjugates and compositions described herein to treat a metabolic disorder such as diabetes (e.g., type 1 diabetes, type 2 diabetes, gestational diabetes);

NBI-6024 (Neurocrine Biosciences, Inc.), a fifteen amino acid peptide, and variants and derivatives thereof, which can be used in the particles, conjugates and compositions described herein to treat a metabolic disorder such as diabetes (e.g., type 1 diabetes, type 2 diabetes, gestational diabetes);

obinepitide (7™ Pharma), a thirty-six amino acid peptide, and variants and derivatives thereof, which can be used in the particles, conjugates and compositions described herein to treat a metabolic disorder such as obesity;

peptide YY (3-36) (MDRNA Inc.), a thirty-four amino acid peptide, and variants and derivatives thereof, which can be used in the particles, conjugates and compositions described herein to treat a metabolic disorder such as obesity;

pramlintide (Symlin™, Amylin Pharmaceuticals), a cyclic thirty four amino acid peptide, and variants and derivatives thereof, which can be used in the particles, conjugates and compositions described herein to treat a metabolic disorder such as diabetes (e.g., type 1 diabetes, type 2 diabetes, gestational diabetes) and obesity;

R-7089 (Roche), and variants and derivatives thereof, which can be used in the particles, conjugates and compositions described herein to treat a metabolic disorder such as diabetes (e.g., type 1 diabetes, type 2 diabetes, gestational diabetes);

semaglutide (NN-9535, Novo Nordisk), and variants and derivatives thereof, which can be used in the particles, conjugates and compositions described herein to treat a metabolic disorder such as diabetes (e.g., type 1 diabetes, type 2 diabetes, gestational diabetes);

SST analog (Merck & Co. Inc.), and variants and derivatives thereof, which can be used in the particles, conjugates and compositions described herein to treat a metabolic disorder such as diabetes (e.g., type 1 diabetes, type 2 diabetes, gestational diabetes);

SUN-E7001 (CS-872, Daiichi Sankyo), a thirty amino acid peptide, and variants and derivatives thereof, which can be used in the particles, conjugates and compositions described herein to treat a metabolic disorder such as diabetes (e.g., type 1 diabetes, type 2 diabetes, gestational diabetes);

taspoglutide (BIM-51077, Roche), a thirty amino acid peptide, and variants and derivatives thereof, which can be used in the particles, conjugates and compositions described herein to treat a metabolic disorder such as diabetes (e.g., type 1 diabetes, type 2 diabetes, gestational diabetes);

tesamorelin (TH-9507, Theratechnologies), a forty-four amino acid peptide, and variants and derivatives thereof, which can be used in the particles, conjugates and compositions described herein to treat a metabolic disorder such as somatotrophin deficiency, muscle wasting and lipodystrophy;

TH-0318 (OctoPlus NV), and variants and derivatives thereof, which can be used in the particles, conjugates and compositions described herein to treat a metabolic disorder such as diabetes (e.g., type 1 diabetes, type 2 diabetes, gestational diabetes);

TKS-1225 (oxyntomodulin, Wyeth), a thirty-seven amino acid peptide, and variants and derivatives thereof, which can be used in the particles, conjugates and compositions described herein to treat a metabolic disorder such as obesity;

TM-30339 (7™ Pharma), and variants and derivatives thereof, which can be used in the particles, conjugates and compositions described herein to treat a metabolic disorder such as obesity;

TT-223 (E1-INT, Eli Lilly & Co.), and variants and derivatives thereof, which can be used in the particles, conjugates and compositions described herein to treat a metabolic disorder such as diabetes (e.g., type 1 diabetes, type 2 diabetes, gestational diabetes);

Unacylated ghrelin (AZP-01, Alize Pharma), a twenty-eight amino acid peptide, and variants and derivatives thereof, which can be used in the particles, conjugates and compositions described herein to treat a metabolic disorder such as diabetes (e.g., type 1 diabetes, type 2 diabetes, gestational diabetes); and

Urocortin II (Neurocrine Biosciences Inc.), a thirty-eight amino acid peptide, and variants and derivatives thereof, which can be used in the particles, conjugates and compositions described herein to treat a metabolic disorder such as obesity.

Cancer

The disclosed therapeutic peptide-polymer conjugates, particles and compositions are useful in treating proliferative disorders, e.g., treating a tumor and metastases thereof wherein the tumor or metastases thereof is a cancer described herein.

The therapeutic peptide can be, e.g., a peptide inhibitor of proliferative signaling (e.g., an inhibitor of mitogenic signaling or a peptide that restores the activity of a tumor suppressor protein such as p53), a cell cycle inhibitor, or an inducer of apoptosis. For example, a peptide inhibitor of proliferative signaling includes peptide inhibitors of Ras activation, peptide inhibitors of MAP kinase, a peptide inhibitor of NF-κB activation, and a peptide inhibitor of c-Myc activation. See, e.g., Bidwell et al. (2009) Expert Opin. Drug Delivery 6(10):1033-1047, the contents of which is incorporated herein by reference.

Examples of therapeutic peptides that can be used in the claimed conjugates, particles and compositions include the following:

A-6 (Angstrom Pharmaceuticals Inc.) an eight amino acid peptide, and variants and derivatives thereof, which can be used in the particles, conjugates and compositions described herein to treat a proliferative disorder, e.g., cancer (e.g., ovarian cancer);

PPI-149 (abarelix, Plenaxis™), a ten amino acid peptide, and variants and derivatives thereof, which can be used in the particles, conjugates and compositions described herein to treat a proliferative disorder such as cancer (e.g., prostate cancer);

ABT-510 (Abbott Laboratories), a nine amino acid peptide, and variants and derivatives thereof, which can be used in the particles, conjugates and compositions described herein to treat a proliferative disorder such as cancer (e.g., lung cancer (e.g., small cell or non-small cell lung cancer), renal cell carcinoma, sarcoma, lymphoma, solid tumors, melanoma and malignant glioma);

ADH-1 (Exherin™, Adherex Technologies), a cyclic five amino acid peptide, and variants and derivatives thereof, which can be used in the particles, conjugates and compositions described herein to treat a proliferative disorder such as cancer (e.g., solid tumors and melanoma);

AEZS-108 (AN-152, ZEN-008, AEtherna Zentaris), a ten amino acid peptide, and variants and derivatives thereof, which can be used in the particles, conjugates and compositions described herein to treat proliferative disorders such as cancer (e.g., endometrial carcinoma, breast cancer, ovarian cancer, and prostate cancer);

afamelanotide (EP-1647, CUV-1647, Melanotan™, Clinuvel Pharmaceuticals, Ltd.) a thirteen amino acid peptide, and variants and derivatives thereof, which can be used in the particles, conjugates and compositions described herein to treat a proliferative disorder such as cancer (e.g., skin cancer);

ambamustine (PTT-119, Abbott Laboratories) a three amino acid peptide, and variants and derivatives thereof, which can be used in the particles, conjugates and compositions described herein to treat a proliferative disorder such as cancer (e.g., lymphoma (e.g., Non-Hodgkin lymphoma) and lung cancer (e.g., small cell or non-small cell lung cancer);

antagonist G (PTL-68001, Arana Therapeutics), a six amino acid peptide, and variants and derivatives thereof, which can be used in the particles, conjugates and compositions described herein to treat a proliferative disorder such as cancer (e.g., lung cancer (e.g., small cell or non-small cell lung cancer), pancreatic cancer and colorectal cancer);

ATN-161 (Attenuon LLC), a five amino acid peptide, and variants and derivatives thereof, which can be used in the particles, conjugates and compositions described herein to treat a proliferative disorder such as cancer (e.g., glioma);

avorelin (EP-23904, Meterelin™, Lutrelin™, Mediolanum Farmaceutici SpA), a nine amino acid peptide, and variants and derivatives thereof, which can be used in the particles, conjugates and compositions described herein to treat a proliferative disorder such as cancer (e.g., prostate cancer and breast cancer);

buserelin (Suprefact™, Suprecur™, Sanofi-Aventis), a ten amino acid peptide, and variants and derivatives thereof, which can be used in the particles, conjugates and compositions described herein to treat proliferative disorders such as cancer (e.g., prostate cancer);

carfilzomib (PR-171, Proteolix Inc.), a four amino acid peptide, and variants and derivatives thereof, which can be used in the particles, conjugates and compositions described herein to treat a proliferative disorder such as cancer (e.g., multiple myeloma, lymphoma, hematological neoplasms, and solid tumors);

CBP-501 (Takeda Pharmaceuticals), a twelve amino acid peptide, and variants and derivatives thereof, which can be used in the particles, conjugates and compositions described herein to treat proliferative disorders such as cancer (e.g., lung cancer (e.g., small cell or non-small cell lung cancer) and mesothelioma);

cemadotin (LU-103793, Abbott Laboratories), a five amino acid peptide, and variants and derivatives thereof, which can be used in the particles, conjugates and compositions described herein to treat proliferative disorders such as cancer;

cetrorelix (NS-75, Cetrotide™, AEterna Zentaris), a ten amino acid peptide, and variants and derivatives thereof, which can be used in the particles, conjugates and compositions described herein to treat proliferative disorders such as benign protastatic hyperplasia, fibroids (e.g., uterine fibroids), cancer (e.g., breast cancer, ovarian cancer, prostate cancer);

chlorotoxin (TM-601, TransMolecular Inc.), a thirty-six amino acid peptide, and variants and derivatives thereof, which can be used in the particles, conjugates and compositions described herein to treat proliferative disorders such as cancer (e.g., glioma);

cilengitide (EMD-121974, EMD-85189), a five amino acid peptide, and variants and derivatives thereof, which can be used in the particles, conjugates and compositions described herein to treat proliferative disorders such as cancer (e.g., lung cancer (e.g., small cell or non-small cell lung cancer), glioblastoma, pancreatic cancer and prostate cancer);

CTCE-9908 (Chemokine Therapeutics Corp.), a seventeen amino acid peptide, and variants and derivatives thereof, which can be used in the particles, conjugates and compositions described herein to treat a proliferative disorder such as cancer;

CVX-045 (Pfizer-Covx), and variants and derivatives thereof, which can be used in the particles, conjugates and compositions described herein to treat a proliferative disorder such as cancer (e.g., a solid tumor);

CVX-060 (Pfizer-Covx), and variants and derivatives thereof, which can be used in the particles, conjugates and compositions described herein to treat a proliferative disorder such as cancer;

degarelix (FE 200486, Ferring Pharmaceuticals), a ten amino acid peptide, and variants and derivatives thereof, which can be used in the particles, conjugates and compositions described herein to treat a proliferative disorder such as cancer (e.g., prostate cancer);

desolorelin (Somagard™, Shire), and variants and derivatives thereof, which can be used in the particles, conjugates and compositions described herein to treat a proliferative disorder such as cancer (e.g., lymphoma (e.g., Non-Hodgkin lymphoma), brain cancer, melanoma);

didemnin B (NSC-325319, PharmaMar), a six amino acid peptide, and variants and derivatives thereof, which can be used in the particles, conjugates and compositions described herein to treat a proliferative disorder such as cancer (e.g., lymphoma (e.g., Non-Hodgkin lymphoma), brain cancer, melanoma);

DRF-7295 (Dabur India Ltd.), and variants and derivatives thereof, which can be used in the particles, conjugates and compositions described herein to treat a proliferative disorder such as cancer (e.g., breast cancer and colorectal cancer);

edotreotide (SMT-487, OctreoTher™, Onaita™, Molecular Insight Pharmaceuticals), a cyclic seven amino acid peptide, and variants and derivatives thereof, which can be used in the particles, conjugates and compositions described herein to treat a proliferative disorder such as cancer;

elisidepsin (PM-02734, Irvalec™, PharmaMar), and variants and derivatives thereof, which can be used in the particles, conjugates and compositions described herein to treat a proliferative disorder such as cancer (e.g., lung cancer (e.g., small cell or non-small cell lung cancer));

EP-100 (Esperance Pharmaceuticals Inc.), a thirty-three amino acid peptide, and variants and derivatives thereof, which can be used in the particles, conjugates and compositions described herein to treat a proliferative disorder such as cancer (e.g., prostate cancer);

ganirelix (Org-37462, RS-26306, Orgalutran™, Antagon™, Schering-Plough Corp), and variants and derivatives thereof, which can be used in the particles, conjugates and compositions described herein to treat a proliferative disorder such as endometriosis and cancer (e.g., prostate cancer and breast cancer);

glutoxim (NOV-002, Pharma Vam), a six amino acid peptide, and variants and derivatives thereof, which can be used in the particles, conjugates and compositions described herein to treat a proliferative disorder such as cancer (e.g., lung cancer (e.g., small cell or non-small cell lung cancer) and ovarian cancer);

goralatide (BIM-32001, Ipsen), a four amino acid peptide, and variants and derivatives thereof, which can be used in the particles, conjugates and compositions described herein to treat a proliferative disorder such as cancer;

goserelin (ICI-118630, AstraZeneca), a ten amino acid peptide, and variants and derivatives thereof, which can be used in the particles, conjugates and compositions described herein to treat a proliferative disorder such as cancer (e.g., prostate cancer, breast cancer, and uterine cancer);

histrelin (Vantas™, Johnson & Johnson), a nine amino acid peptide, and variants and derivatives thereof, which can be used in the particles, conjugates and compositions described herein to treat a proliferative disorder such as cancer (e.g., prostate cancer);

labradimil (RMP-7, Cereport™, Johnson & Johnson), a nine amino acid peptide, and variants and derivatives thereof, which can be used in the particles, conjugates and compositions described herein to treat a proliferative disorder such as cancer (e.g., glioma and brain cancer);

leuprolide (Lupron™, Prostap™, Leuplin™, Enantone™, Takeda Pharmaceuticals), a nine amino acid peptide, and variants and derivatives thereof, which can be used in the particles, conjugates and compositions described herein to treat a proliferative disorder such as fibroids (e.g., uterine fibroids) and cancer (e.g., prostate cancer);

LY-2510924 (AVE-0010, Sanofi-Aventis), a cyclic amino acid peptide, and variants and derivatives thereof, which can be used in the particles, conjugates and compositions described herein to treat a proliferative disorder such as and cancer (e.g., breast cancer);

mifamurtide (Junovan™, Metpact™, Takeda Pharmaceuticals), a three amino acid peptide, and variants and derivatives thereof, which can be used in the particles, conjugates and compositions described herein to treat a proliferative disorder such as cancer (e.g., osteosarcoma);

met-enkephalin (INNO-105, Innovive Pharmaceuticals Inc.), a five amino acid peptide, and variants and derivatives thereof, which can be used in the particles, conjugates and compositions described herein to treat a proliferative disorder such as cancer (e.g., a solid tumor, pancreatic cancer);

muramyk tripeptide (Novartis), a three amino acid peptide, and variants and derivatives thereof, which can be used in the particles, conjugates and compositions described herein to treat a proliferative disorder such as cancer;

nafarelin (RS-94991, Samynarel™, Nasanyl™, Synarel™, Synareia™, Roche), and variants and derivatives thereof, which can be used in the particles, conjugates and compositions described herein to treat a proliferative disorder such as endometriosis and cancer (e.g., prostate cancer and breast cancer);

octreotide (SMS-201-995, Sandostatin™, Novartis), and variants and derivatives thereof, which can be used in the particles, conjugates and compositions described herein to treat a proliferative disorder such as benign prostatic hyperplasia and cancer (e.g., prostate cancer);

ozarelix (D-63153, SPI-153, Spectrum Pharmaceuticals) a ten amino acid peptide, and variants and derivatives thereof, which can be used in the particles, conjugates and compositions described herein to treat a proliferative disorder such as benign prostatic hyperplasia and cancer (e.g., prostate cancer);

POL-6326 (Polyphor), and variants and derivatives thereof, which can be used in the particles, conjugates and compositions described herein to treat a proliferative disorder such as cancer;

ramorelix (Hoe-013, Sanofi Aventis), a nine amino acid peptide, and variants and derivatives thereof, which can be used in the particles, conjugates and compositions described herein to treat a proliferative disorder such as fibroids (e.g., uterine fibroids) and cancer (e.g., prostate cancer);

RC-3095 (AEterna Zentaris), a six amino acid peptide, and variants and derivatives thereof, which can be used in the particles, conjugates and compositions described herein to treat a proliferative disorder such as cancer (e.g., a solid tumor);

Re-188-P-2045 (P2045, Neotide™, Bryan Oncor), an eleven amino acid peptide, and variants and derivatives thereof, which can be used in the particles, conjugates and compositions described herein to treat a proliferative disorder such as cancer (e.g., lung cancer (e.g., small cell or non-small cell lung cancer));

romurtide (DJ-7041, Nopia™, Muroctasin™, Daiichi Sankyo), a two amino acid peptide, and variants and derivatives thereof, which can be used in the particles, conjugates and compositions described herein to treat a proliferative disorder such as cancer;

YHI-501 (TZT-1027, Yakult Honsha KK), a two amino acid peptide, and variants and derivatives thereof, which can be used in the particles, conjugates and compositions described herein to treat a proliferative disorder such as cancer (e.g., solid tumors);

SPI-1620 (Spectrum Pharmaceuticals), a fourteen amino acid peptide, and variants and derivatives thereof, which can be used in the particles, conjugates and compositions described herein to treat a proliferative disorder such as cancer (e.g., solid tumors);

tabilautide (RP-56142, Sanofi Aventis), a three amino acid peptide, and variants and derivatives thereof, which can be used in the particles, conjugates and compositions described herein to treat a proliferative disorder such as cancer;

TAK-448 (Takeda Pharmaceuticals), and variants and derivatives thereof, which can be used in the particles, conjugates and compositions described herein to treat a proliferative disorder such as cancer (e.g., prostate cancer);

TAK-683 (Takeda Pharmaceuticals), and variants and derivatives thereof, which can be used in the particles, conjugates and compositions described herein to treat a proliferative disorder such as cancer (e.g., prostate cancer);

tasidotin (ILX-651, BSF-223651, Genzyme), a five amino acid peptide, and variants and derivatives thereof, which can be used in the particles, conjugates and compositions described herein to treat a proliferative disorder such as cancer (e.g., melanoma, prostate cancer and lung cancer (e.g., small cell or non-small cell lung cancer));

teverelix (EP-24332, Antarelix™, Ardana Biosciences), a ten amino acid peptide, and variants and derivatives thereof, which can be used in the particles, conjugates and compositions described herein to treat a proliferative disorder such as endometriosis, benign prostatic hyperplasia and cancer (e.g., prostate cancer);

tigapotide (PCK-3145, Kotinos Pharmaceuticals), a fifteen amino acid peptide, and variants and derivatives thereof, which can be used in the particles, conjugates and compositions described herein to treat a proliferative disorder such as endometriosis, benign prostatic hyperplasia and cancer (e.g., prostate cancer);

thymalfasin (Zadaxin™, Timosa™, Thymalfasin™, SciClone Pharmaceuticals), a twenty-eight amino acid peptide, and variants and derivatives thereof, which can be used in the particles, conjugates and compositions described herein to treat a proliferative disorder such as cancer (e.g., melanoma, lung cancer (e.g., small cell or non-small cell lung cancer) and carcinoma (e.g., hepatocellular carcinoma));

TLN-232 (CAP-232, TT-232, Thallion Pharmaceuticals), a seven amino acid peptide, and variants and derivatives thereof, which can be used in the particles, conjugates and compositions described herein to treat a proliferative disorder such as endometriosis, benign prostatic hyperplasia and cancer;

triptorelin (WY-42462, Debiopharma), a ten amino acid peptide, and variants and derivatives thereof, which can be used in the particles, conjugates and compositions described herein to treat a proliferative disorder such as endometriosis, fibroids (e.g., uterine fibroids), benign prostatic hyperplasia and cancer (e.g., prostate cancer and breast cancer);

tyroserleutide (CMS-024, China Medical System), a three amino acid peptide, and variants and derivatives thereof, which can be used in the particles, conjugates and compositions described herein to treat a proliferative disorder such as cancer (e.g., liver cancer (e.g., hepatocellular carcinoma); and

tyroservatide (CMS-024-02, China Medical Systems), a three amino acid peptide, and variants and derivatives thereof, which can be used in the particles, conjugates and compositions described herein to treat a proliferative disorder such as cancer (e.g., lung cancer (e.g., small cell or non-small cell lung cancer)).

Cardiovascular Disease

The disclosed therapeutic peptide-polymer conjugates, particles and compositions may be useful in the prevention and treatment of cardiovascular disease.

Exemplary therapeutic peptides that can be used in the disclosed conjugates, particles and compositions include the following:

AC-2592 (Betatropin™, Amylin Pharmaceuticals), a thirty amino acid peptide, and variants and derivatives thereof, which can be used in the particles, conjugates and compositions described herein to treat a cardiovascular disorder such as heart failure;

AC-625 (Amylin Pharmaceuticals), a peptide, and variants and derivatives thereof, which can be used in the particles, conjugates and compositions described herein to treat a cardiovascular disorder such as hypertension;

Anaritide (Auriculin™, Johnson & Johnson), a cyclic twenty-five amino acid peptide, and variants and derivatives thereof, which can be used in the particles, conjugates and compositions described herein to treat a cardiovascular disorder such as renal failure, heart failure, and hypertension;

APL-180 (Novartis), a peptide, and variants and derivatives thereof, which can be used in the particles, conjugates and compositions described herein to treat a cardiovascular disorder such as coronary disorder;

Atriopeptin (Astellas Pharma), a twenty-five amino acid peptide, and variants and derivatives thereof, which can be used in the particles, conjugates and compositions described herein to treat a cardiovascular disorder;

BGC-728 (BTG plc), a cyclic peptide, and variants and derivatives thereof, which can be used in the particles, conjugates and compositions described herein to treat a cardiovascular disorder such as myocardial infarction and cerebrovascular ischemia;

Carperitide (SUN-4936, HANP™, Daiichi Sankyo), a cyclic peptide, and variants and derivatives thereof, which can be used in the particles, conjugates and compositions described herein to treat a cardiovascular disorder such as heart failure;

CD-NP (Nile Therapeutics), a forty-one amino acid peptide, and variants and derivatives thereof, which can be used in the particles, conjugates and compositions described herein to treat a cardiovascular disorder such as heart failure;

CG-77×56 (Cardeva™, Teva Pharmaceuticals), a peptide, and variants and derivatives thereof, which can be used in the particles, conjugates and compositions described herein to treat a cardiovascular disorder such as heart failure;

D-4F (APP-018, Novartis), an eighteen amino acid peptide, and variants and derivatives thereof, which can be used in the particles, conjugates and compositions described herein to treat a cardiovascular disorder such as atherosclerosis;

Danegaptide (ZP-1609, WAY-261134, GAP-134, Zealand Pharma), a two amino acid peptide, and variants and derivatives thereof, which can be used in the particles, conjugates and compositions described herein to treat a cardiovascular disorder such as heart arrhythmia;

DMP-728 (DU-728, Bristol-Myers Squibb), a cyclic three amino acid peptide, and variants and derivatives thereof, which can be used in the particles, conjugates and compositions described herein to treat a cardiovascular disorder such as thrombosis (e.g., coronary thrombosis);

Efegatran (LY-294468, Eli Lilly and Co.), a three amino acid peptide, and variants and derivatives thereof, which can be used in the particles, conjugates and compositions described herein to treat a cardiovascular disorder such as myocardial infarction and thrombosis (e.g., coronary thrombosis);

EMD-73495 (Merck kGaA), a peptide, and variants and derivatives thereof, which can be used in the particles, conjugates and compositions described herein to treat a cardiovascular disorder;

Eptifibatide (C68-22, Integrelin™, Integrilin™, Takeda Pharmaceuticals), a cyclic six amino acid peptide, and variants and derivatives thereof, which can be used in the particles, conjugates and compositions described herein to treat a cardiovascular disorder such as acute coronary syndrome, myocardial infarction, and unstable angina pectoris;

ET-642 (RLT-peptide, Pfizer), a twenty-two amino acid peptide, and variants and derivatives thereof, which can be used in the particles, conjugates and compositions described herein to treat a cardiovascular disorder such as atherosclerosis;

FE 202158 (Ferring Pharmaceuticals), a cyclic nine amino acid peptide, and variants and derivatives thereof, which can be used in the particles, conjugates and compositions described herein to treat a cardiovascular disorder such as vasodilatory hypotension (e.g., sepsis and intradialytic hypotension);

FX-06 (Ikaria), a peptide, and variants and derivatives thereof, which can be used in the particles, conjugates and compositions described herein to treat a cardiovascular disorder such as reperfusion injury;

Icrocaptide (ITF-1697, Italfarmaco), a four amino acid peptide, and variants and derivatives thereof, which can be used in the particles, conjugates and compositions described herein to treat a cardiovascular disorder such as respiratory distress syndrome;

KAI-1455 (KAI Pharmaceuticals), a twenty amino acid peptide, and variants and derivatives thereof, which can be used in the particles, conjugates and compositions described herein to treat a cardiovascular disorder such as cardiovascular surgery cytoprotection;

KAI-9803 (Bristo-Myers Squibb), a twenty-three amino acid peptide, and variants and derivatives thereof, which can be used in the particles, conjugates and compositions described herein to treat a cardiovascular disorder such as myocardial infarction, reperfusion injury, and coronary artery disease;

L-346670 (Merck & Co. Inc.), a cyclic twenty-six amino acid peptide, and variants and derivatives thereof, which can be used in the particles, conjugates and compositions described herein to treat a cardiovascular disorder such as hypertension;

L-364343 (Merck & Co. Inc.), a cyclic twenty-nine amino acid peptide, and variants and derivatives thereof, which can be used in the particles, conjugates and compositions described herein to treat a cardiovascular disorder such as hypertension;

LSI-518P (Lipid Sciences Inc.), a peptide, and variants and derivatives thereof, which can be used in the particles, conjugates and compositions described herein to treat a cardiovascular disorder;

Nesiritide (Noratak™, Natrecor™, Johnson & Johnson), a thirty-two amino acid peptide, and variants and derivatives thereof, which can be used in the particles, conjugates and compositions described herein to treat a cardiovascular disorder such as heart failure;

Peptide rennin inhibitor (Pfizer), a peptide, and variants and derivatives thereof, which can be used in the particles, conjugates and compositions described herein to treat a cardiovascular disorder;

PL-3994 (Palatin Technologies), a fifteen amino acid peptide, and variants and derivatives thereof, which can be used in the particles, conjugates and compositions described herein to treat a cardiovascular disorder such as hypertension and heart failure;

Rotigaptide (ZP-123, GAP-486, Zealand Pharma), a six amino acid peptide, and variants and derivatives thereof, which can be used in the particles, conjugates and compositions described herein to treat a cardiovascular disorder such as ventricular arrhythmia and atrial fibrillation;

Saralasin (P-113, Sarenin™, Procter & Gamble), an eight amino acid peptide, and variants and derivatives thereof, which can be used in the particles, conjugates and compositions described herein to treat a cardiovascular disorder;

SKF-105494 (GlaxoSmithKline), a cyclic seven amino acid peptide, and variants and derivatives thereof, which can be used in the particles, conjugates and compositions described herein to treat a cardiovascular disorder such as hypertension;

Terlakiren (CP-80794, Pfizer), a two amino acid peptide, and variants and derivatives thereof, which can be used in the particles, conjugates and compositions described herein to treat a cardiovascular disorder such as hypertension;

Thymalfasin (Zadaxin™, Timosa™, Thymalfasin™, SciClone Pharmaceuticals), a twenty-eight amino acid peptide, and variants and derivatives thereof, which can be used in the particles, conjugates and compositions described herein to treat a cardiovascular disorder such as angiogenesis disorder;

Tridecactide (AP-214, Action Pharma), a ten amino acid peptide, and variants and derivatives thereof, which can be used in the particles, conjugates and compositions described herein to treat a cardiovascular disorder such as reperfusion injury and renal disease;

Ularitide (CDD-95-126, ESP-305, CardioBiss™, Nephrobiss™, EKR Therapeutics), a cyclic thirty-two amino acid peptide, and variants and derivatives thereof, which can be used in the particles, conjugates and compositions described herein to treat a cardiovascular disorder such as heart failure and renal failure;

Urocortin II (Neurocrine Biosciences Inc.), a thirty-eight amino acid peptide, and variants and derivatives thereof, which can be used in the particles, conjugates and compositions described herein to treat a cardiovascular disorder such as heart failure; and

ZP-120 (Zealand Pharma), a twelve amino acid peptide, and variants and derivatives thereof, which can be used in the particles, conjugates and compositions described herein to treat a cardiovascular disorder such as isolated systolic hypertension and heart failure.

Infectious Disease

The conjugates, particles and compositions described herein can include a peptide that treats or prevents infectious disease. Exemplary therapeutic peptides that can be used in the disclosed conjugates, particles and compositions include the following:

Albuvirtide (Frontier Biotechnologies), a peptide, and variants and derivatives thereof, which can be used in the particles, conjugates and compositions described herein to treat a microbial disorder or viral disorder such as HIV infection;

ALG-889 (Allergene Inc.), a sixteen amino acid peptide, and variants and derivatives thereof, which can be used in the particles, conjugates and compositions described herein to treat a microbial disorder or viral disorder such as HIV infection and immune disorder;

Alloferon (Allokine-alpha™, EntoPharm Co. Ltd.), a thirteen amino acid peptide, and variants and derivatives thereof, which can be used in the particles, conjugates and compositions described herein to treat a microbial disorder or viral disorder such as hepatitis B virus infection, hepatitis C virus infection, herpesvirus infection, and cancer;

ALX-40-AC (NPS Pharmaceuticals), a nine amino acid peptide, and variants and derivatives thereof, which can be used in the particles, conjugates and compositions described herein to treat a microbial disorder or viral disorder such as HIV infection;

CB-182804 (Cubist Pharmaceuticals), a peptide, and variants and derivatives thereof, which can be used in the particles, conjugates and compositions described herein to treat a microbial disorder or viral disorder such as multidrug-resistant Gram negative bacterial infection;

CB-183315 (Cubist Pharmaceuticals), a peptide, and variants and derivatives thereof, which can be used in the particles, conjugates and compositions described herein to treat a microbial disorder or viral disorder such as Clostridium difficile-associated diarrhea;

CZEN-002 (Migami), a polymeric eight amino acid peptide, and variants and derivatives thereof, which can be used in the particles, conjugates and compositions described herein to treat a microbial disorder or viral disorder such as vulvovaginal candidiasis;

Enfuvirtide (T-20, Fuzeon™, Roche), a thirty-six amino acid peptide, and variants and derivatives thereof, which can be used in the particles, conjugates and compositions described herein to treat a microbial disorder or viral disorder such as HIV infection;

Glucosamyl muramyl tripeptide (Theramide™, DOR BioPharma Inc.), a three amino acid peptide, and variants and derivatives thereof, which can be used in the particles, conjugates and compositions described herein to treat a microbial disorder or viral disorder such as herpesvirus infection, postoperative infections, psoriasis, respiratory tract disorders (e.g., lung disorders), and tuberculosis;

GMDP (Likopid™, Licopid™, Arana Therapeutics), a two amino acid peptide, and variants and derivatives thereof, which can be used in the particles, conjugates and compositions described herein to treat a microbial disorder or viral disorder such as herpesvirus infection, postoperative infections, psoriasis, respiratory tract disorders (e.g., lung disorders), and tuberculosis;

Golotimod (SCV-07, SciClone Pharmaceuticals), a two amino acid peptide, and variants and derivatives thereof, which can be used in the particles, conjugates and compositions described herein to treat a microbial disorder or viral disorder such as hepatitis C, viral infection, and tuberculosis;

GPG-NH2 (Tripep), a three amino acid peptide, and variants and derivatives thereof, which can be used in the particles, conjugates and compositions described herein to treat a microbial disorder or viral disorder such as HIV infection;

hLF(1-11) (AM-Pharma Holding BV), an eleven amino acid peptide, and variants and derivatives thereof, which can be used in the particles, conjugates and compositions described herein to treat a microbial disorder or viral disorder such as bacterial infection, mycoses, bacteremia, and candidemia;

IMX-942 (Inimex Pharmaceuticals), a peptide, and variants and derivatives thereof, which can be used in the particles, conjugates and compositions described herein to treat a microbial disorder or viral disorder such as hospital-acquired bacterial infections;

Iseganan (IB-367, Ardea Biosciences Inc.), a cyclic sixteen amino acid peptide, and variants and derivatives thereof, which can be used in the particles, conjugates and compositions described herein to treat a microbial disorder or viral disorder such as stomatitis and nosocomial pneumonia;

Murabutide (VA-101, CY-220, Sanofi-Aventis), a two amino acid peptide, and variants and derivatives thereof, which can be used in the particles, conjugates and compositions described herein to treat a microbial disorder or viral disorder such as hepatitis virus infection and HIV infection;

Neogen (Neogen™, Immunotech Developments), a peptide, and variants and derivatives thereof, which can be used in the particles, conjugates and compositions described herein to treat a microbial disorder or viral disorder such as viral infection, bacterial infection, and hemopoietic disorder;

NP-213 (Novexatin™, NovaBiotics), a cyclic amino acid peptide, and variants and derivatives thereof, which can be used in the particles, conjugates and compositions described herein to treat a microbial disorder or viral disorder such as onychomycosis;

Oglufanide (IM-862, Implicit Bioscience), a two amino acid peptide, and variants and derivatives thereof, which can be used in the particles, conjugates and compositions described herein to treat a microbial disorder or viral disorder such as hepatitis C virus infection;

Omiganan (CPI-226, Omigard™, Migenix Inc.), a twelve amino acid peptide, and variants and derivatives thereof, which can be used in the particles, conjugates and compositions described herein to treat a microbial disorder or viral disorder such as catheter infection and rosacea;

OP-145 (OctoPlus NV), a peptide, and variants and derivatives thereof, which can be used in the particles, conjugates and compositions described herein to treat a microbial disorder or viral disorder such as otitis;

p-1025 (Sinclair Pharma plc), a nineteen amino acid peptide, and variants and derivatives thereof, which can be used in the particles, conjugates and compositions described herein to treat a microbial disorder or viral disorder such as dental caries;

P-113 (PAC-113, HistaWash™, Histat gingivitis gel™, Histat periodontal Wafer™, Pacgen Biopharmaceuticals Corp.), a twelve amino acid peptide, and variants and derivatives thereof, which can be used in the particles, conjugates and compositions described herein to treat a microbial disorder or viral disorder such as Candida albicans infection and gingivitis;

Pep-F (5K, Milkhaus Laboratory Inc.), a peptide, and variants and derivatives thereof, which can be used in the particles, conjugates and compositions described herein to treat a microbial disorder or viral disorder such as herpesvirus infection;

R-15-K (BlockAide/CR™, Adventrx Pharmaceuticals Inc.), a fifteen amino acid peptide, and variants and derivatives thereof, which can be used in the particles, conjugates and compositions described herein to treat a microbial disorder or viral disorder such as HIV infection;

Sifuvirtide (FusoGen Pharmaceuticals Inc.), a thirty-six amino acid peptide, and variants and derivatives thereof, which can be used in the particles, conjugates and compositions described herein to treat a microbial disorder or viral disorder such as HIV infection;

SPC-3 (Columbia Laboratories), a polymeric fifty-six amino acid peptide, and variants and derivatives thereof, which can be used in the particles, conjugates and compositions described herein to treat a microbial disorder or viral disorder such as HIV infection;

Thymalfasin (Zadaxin™, Timosa™, Thymalfasin™, SciClone Pharmaceuticals), a twenty-eight amino acid peptide, and variants and derivatives thereof, which can be used in the particles, conjugates and compositions described herein to treat a microbial disorder or viral disorder such as cancer (e.g., heptocellular carcinoma), hepatitis B virus infection, hepatitis C virus infection, HIV infection, influenza virus infection, aspergillus infection, and wound healing;

Thymonoctan (FCE-25388, Pfizer), an eight amino acid peptide, and variants and derivatives thereof, which can be used in the particles, conjugates and compositions described herein to treat a microbial disorder or viral disorder such as hepatitis virus infection and HIV infection;

Thymopentin (TP-5, Timunox™, Johnson & Johnson), a five amino acid peptide, and variants and derivatives thereof, which can be used in the particles, conjugates and compositions described herein to treat a microbial disorder or viral disorder such as lung infection and HIV infection;

Tifuvirtide (R-724, T-1249, Roche), a thirty-nine amino acid peptide, and variants and derivatives thereof, which can be used in the particles, conjugates and compositions described herein to treat a microbial disorder or viral disorder such as HIV infection;

TRI-1144 (Trimeris Inc.), a thirty-eight amino acid peptide, and variants and derivatives thereof, which can be used in the particles, conjugates and compositions described herein to treat a microbial disorder or viral disorder such as HIV infection;

VIR-576 (Pharis Biotec), a forty amino acid peptide, and variants and derivatives thereof, which can be used in the particles, conjugates and compositions described herein to treat a microbial disorder or viral disorder such as HIV infection; and

XOMA-629 (XOMA Ltd.), a fifteen amino acid peptide, and variants and derivatives thereof, which can be used in the particles, conjugates and compositions described herein to treat a microbial disorder or viral disorder such as acne, Staphylococcus aureus infection, and impetigo.

Allergy, Inflammatory and Autoimmune Disorders

The conjugates, particles and compositions described herein can include a peptide that treats or prevents allergy, inflammatory and/or autoimuune disorders. Exemplary therapeutic peptides that can be used in the disclosed conjugates, particles and compositions include the following:

A-623 (AMG-623, Anthera Pharmaceuticals), a peptide, and variants and derivatives thereof, which can be used in the particles, conjugates and compositions described herein to treat an allergy, inflammatory disorder, or immune disorder such as lupus erythematosus and chronic lymphocytic leukemia;

AG-284 (AnergiX.MS™, GlaxoSmithKline), a nineteen amino acid peptide, and variants and derivatives thereof, which can be used in the particles, conjugates and compositions described herein to treat an allergy, inflammatory disorder, or immune disorder such as multiple sclerosis;

AI-502 (AutoImmune), a peptide, and variants and derivatives thereof, which can be used in the particles, conjugates and compositions described herein to treat an allergy, inflammatory disorder, or immune disorder such as transplant rejection;

Allotrap 2702 (B-2702, Allotrap 2702, Genzyme), a ten amino acid peptide, and variants and derivatives thereof, which can be used in the particles, conjugates and compositions described herein to treat an allergy, inflammatory disorder, or immune disorder such as transplant rejection;

AZD-2315 (AstraZeneca), an eight amino acid peptide, and variants and derivatives thereof, which can be used in the particles, conjugates and compositions described herein to treat an allergy, inflammatory disorder, or immune disorder such as rheumatoid arthritis;

Cnsnqic-Cyclic (802-2, Adeona Pharmaceuticals), a cyclic five amino acid peptide, and variants and derivatives thereof, which can be used in the particles, conjugates and compositions described herein to treat an allergy, inflammatory disorder, or immune disorder such as Factor VIII deficiency, multiple sclerosis, and graft versus host disease;

Delmitide (RDP-58, Genzyme), a ten amino acid peptide, and variants and derivatives thereof, which can be used in the particles, conjugates and compositions described herein to treat an allergy, inflammatory disorder, or immune disorder such as inflammatory bowel disease, ulcerative colitis, and Crohn's disease;

Dirucotide (MBP-8298, Eli Lilly and Co.), a seventeen amino acid peptide, and variants and derivatives thereof, which can be used in the particles, conjugates and compositions described herein to treat an allergy, inflammatory disorder, or immune disorder such as multiple sclerosis;

Disitertide (NAFB-001, P-144, ISDIN SA), a cyclic fourteen amino acid peptide, and variants and derivatives thereof, which can be used in the particles, conjugates and compositions described herein to treat an allergy, inflammatory disorder, or immune disorder such as scleroderma;

dnaJP1 (AT-001, Adeona Pharmaceuticals), a fifteen amino acid peptide, and variants and derivatives thereof, which can be used in the particles, conjugates and compositions described herein to treat an allergy, inflammatory disorder, or immune disorder such as rheumatoid arthritis;

Edratide (TV-4710, Teva Pharmaceuticals), a twenty amino acid peptide, and variants and derivatives thereof, which can be used in the particles, conjugates and compositions described herein to treat an allergy, inflammatory disorder, or immune disorder such as systemic lupus erythematosus;

F-991 (Clinquest Inc.), a nine amino acid peptide, and variants and derivatives thereof, which can be used in the particles, conjugates and compositions described herein to treat an allergy, inflammatory disorder, or immune disorder such as allergic asthma and skin disorder;

FAR-404 (Enkorten™, Farmacija doo), a peptide, and variants and derivatives thereof, which can be used in the particles, conjugates and compositions described herein to treat an allergy, inflammatory disorder, or immune disorder such as functional bowel disorder, multiple sclerosis, rheumatoid arthritis, asthma, and systemic lupus erythematosus;

Glaspimod (SKF-107647, GlaxoSmithKline), an eight amino acid peptide, and variants and derivatives thereof, which can be used in the particles, conjugates and compositions described herein to treat an allergy, inflammatory disorder, or immune disorder such as leucopenia drug induced fungal infection, immune disorder, viral infection, bacterial infection, and immune deficiency;

Glatiramer (COP-1, Copaxone™, Teva Pharmaceuticals), a peptide, and variants and derivatives thereof, which can be used in the particles, conjugates and compositions described herein to treat an allergy, inflammatory disorder, or immune disorder such as glaucoma, Huntington's chorea, motor neuron disease, multiple sclerosis, and neurodegenerative disease;

Glucosamyl muramyl tripeptide (Theramide™, DOR BioPharma Inc.), a three amino acid peptide, and variants and derivatives thereof, which can be used in the particles, conjugates and compositions described herein to treat an allergy, inflammatory disorder, or immune disorder such as herpesvirus infection, postoperative infections, psoriasis, respiratory tract disorders (e.g., lung disorders), and tuberculosis;

GMDP (Likopid™, Licopid™, Arana Therapeutics), a two amino acid peptide, and variants and derivatives thereof, which can be used in the particles, conjugates and compositions described herein to treat an allergy, inflammatory disorder, or immune disorder such as herpesvirus infection, postoperative infections, psoriasis, respiratory tract disorders (e.g., lung disorders), and tuberculosis;

Icatibant (JE-049, HOE-140, Firazyr™, Shire), an eight amino acid peptide, and variants and derivatives thereof, which can be used in the particles, conjugates and compositions described herein to treat an allergy, inflammatory disorder, or immune disorder such as hereditary angioedema, rhinitis, asthma, osteoarthritis, pain, and liver cirrhosis;

IPP-201101 (Lupuzor™, ImmuPharma Ltd.), a peptide, and variants and derivatives thereof, which can be used in the particles, conjugates and compositions described herein to treat an allergy, inflammatory disorder, or immune disorder such as systemic lupus erythematosus;

MS peptide (Briana Bio-Tech Inc.), a peptide, and variants and derivatives thereof, which can be used in the particles, conjugates and compositions described herein to treat an allergy, inflammatory disorder, or immune disorder such as multiple sclerosis;

Org-42982 (AG-4263, AnergiX.RA™, GlaxoSmithKline), a thirteen amino acid peptide, and variants and derivatives thereof, which can be used in the particles, conjugates and compositions described herein to treat an allergy, inflammatory disorder, or immune disorder such as rheumatoid arthritis;

Pentigetide (TA-521, Pentyde™, Bausch & Lomb), a five amino acid peptide, and variants and derivatives thereof, which can be used in the particles, conjugates and compositions described herein to treat an allergy, inflammatory disorder, or immune disorder such as allergic rhinitis and allergic conjunctivitis;

PI-0824 (Genzyme), a nineteen amino acid peptide, and variants and derivatives thereof, which can be used in the particles, conjugates and compositions described herein to treat an allergy, inflammatory disorder, or immune disorder such as pemphigus vulgaris;

PI-2301 (Peptimmune), a peptide, and variants and derivatives thereof, which can be used in the particles, conjugates and compositions described herein to treat an allergy, inflammatory disorder, or immune disorder such as multiple sclerosis;

PLD-116 (Barr Pharmaceuticals Inc.), a fifteen amino acid peptide, and variants and derivatives thereof, which can be used in the particles, conjugates and compositions described herein to treat an allergy, inflammatory disorder, or immune disorder such as ulcerative colitis;

PMX-53 (Arana Therapeutics), a cyclic six amino acid peptide, and variants and derivatives thereof, which can be used in the particles, conjugates and compositions described herein to treat an allergy, inflammatory disorder, or immune disorder such as inflammation, rheumatoid arthritis, and psoriasis;

PTL-0901 (Acambis plc), a nine amino acid peptide, and variants and derivatives thereof, which can be used in the particles, conjugates and compositions described herein to treat an allergy, inflammatory disorder, or immune disorder such as allergic rhinitis;

RA peptide (Acambis plc), a four amino acid peptide, and variants and derivatives thereof, which can be used in the particles, conjugates and compositions described herein to treat an allergy, inflammatory disorder, or immune disorder such as rheumatoid arthritis;

TCMP-80 (Elan Corp.), a two amino acid peptide, and variants and derivatives thereof, which can be used in the particles, conjugates and compositions described herein to treat an allergy, inflammatory disorder, or immune disorder;

Thymodepressin (Immunotech Developments), a two amino acid peptide, and variants and derivatives thereof, which can be used in the particles, conjugates and compositions described herein to treat an allergy, inflammatory disorder, or immune disorder such as recurring autoimmune cytopenia (1, 2, 3 lineage), hypoplastic anemia, rheumatoid arthritis, and psoriasis;

Thymopentin (TP-5, Timunox™, Johnson & Johnson), a five amino acid peptide, and variants and derivatives thereof, which can be used in the particles, conjugates and compositions described herein to treat an allergy, inflammatory disorder, or immune disorder such as lung infection, rheumatoid arthritis, HIV infection, and primary immunodeficiencies;

Tiplimotide (NBI-5788, Neurocrine Biosciences Inc.), a seventeen amino acid peptide, and variants and derivatives thereof, which can be used in the particles, conjugates and compositions described herein to treat an allergy, inflammatory disorder, or immune disorder such as multiple sclerosis;

Ularitide (CDD-95-126, ESP-305, CardioBiss™, Nephrobiss™, EKR Therapeutics), a cyclic thirty-two amino acid peptide, and variants and derivatives thereof, which can be used in the particles, conjugates and compositions described herein to treat an allergy, inflammatory disorder, or immune disorder such as asthma; and

ZP-1848 (Zealand Pharma), a peptide, and variants and derivatives thereof, which can be used in the particles, conjugates and compositions described herein to treat an allergy, inflammatory disorder, or immune disorder.

Nephrology

The disclosed therapeutic peptide-polymer conjugates, particles and compositions are useful in treating kidney disorders, e.g., a kidney disorder described herein.

The therapeutic peptide can be, e.g., a peptide agonist of GHRH receptor, a peptide agonist of ANP receptor, a peptide agonist of AVP receptora peptide agonist of CALC receptor, a peptide agonist of CRH receptor, a peptide agonist of SST receptor, a peptide agonist of IL-2 receptor, and a peptide agonist of MC receptor.

Examples of therapeutic peptides that can be used in the claimed conjugates, particles and compositions include the following:

AKL-0707 (Aleka Pharma) a twenty-nine amino acid peptide, and variants and derivatives thereof, which can be used in the particles, conjugates and compositions described herein to treat a kidney disorder, e.g., kidney dysfunction associated with a lipid metabolism disorder;

Aniritide (Johnson & Johnson) a twenty-five amino acid cyclic peptide, and variants and derivatives thereof, which can be used in the particles, conjugates and compositions described herein to treat a kidney disorder, e.g., renal failure;

BIM-44002 (Ipsen) a twenty-eight amino acid peptide, and variants and derivatives thereof, which can be used in the particles, conjugates and compositions described herein to treat a kidney disorder, e.g., renal failure, e.g., hypercalcemia associated with renal failure;

Human Calcitonin (also referred to as Cibacalcin®) (Novartis) a thirty-two amino acid peptide, and variants and derivatives thereof, which can be used in the particles, conjugates and compositions described herein to treat a kidney disorder, e.g., renal failure, e.g., hypercalcemia associated with renal failure;

Salmon Calcitonin (also referred to as Calcimar®) (Sanofi-Aventis) a thirty-two amino acid cyclic peptide, and variants and derivatives thereof, which can be used in the particles, conjugates and compositions described herein to treat a kidney disorder, e.g., renal failure, e.g., hypercalcemia associated with renal failure;

C-peptide (also referred to as SPM-933) (Cebix) a thirty-one amino acid linear peptide, and variants and derivatives thereof, which can be used in the particles, conjugates and compositions described herein to treat a kidney disorder, e.g., nephropathy, e.g., diabetic nephropathy;

Desmopressin (also referred to as Minirin®, DDAVP®, or Octostim®) (Ferring Pharmaceuticals) a nine amino acid cyclic peptide, and variants and derivatives thereof, which can be used in the particles, conjugates and compositions described herein to treat a kidney disorder, e.g., nephropathy, e.g., diabetic nephropathy;

DG-3173 (also referred to as PTR-3173 or Somatoprim®) (DeveloGen) an eight amino acid cyclic peptide, and variants and derivatives thereof, which can be used in the particles, conjugates and compositions described herein to treat a kidney disorder, e.g., nephropathy, e.g., diabetic nephropathy;

EA-230 (Exponential Biotherapies) a four amino acid linear peptide, and variants and derivatives thereof, which can be used in the particles, conjugates and compositions described herein to treat a kidney disorder, e.g., renal failure;

Elcatonin (also referred to as Sidinuo® or Elcitonin®) (Asahi Kasei Pharma) a thirty-one amino acid cyclic peptide, and variants and derivatives thereof, which can be used in the particles, conjugates and compositions described herein to treat a kidney disorder, e.g., renal failure, e.g., hypercalcemia associated with renal failure;

Lypressin (also referred to as Diapid®) (Novartis) a nine amino acid cyclic peptide, and variants and derivatives thereof, which can be used in the particles, conjugates and compositions described herein to treat a kidney disorder, e.g., diabetes insipidus;

Terlipressin (also referred to as Glypressin®) (Ferring Pharmaceuticals) a twelve amino acid cyclic peptide, and variants and derivatives thereof, which can be used in the particles, conjugates and compositions described herein to treat a kidney disorder, e.g., hepatorenal syndrome;

Tridecactide (also referred to as AP-214) (Action Pharma) a ten amino acid linear peptide, and variants and derivatives thereof, which can be used in the particles, conjugates and compositions described herein to treat a kidney disorder; and

Ularitide (also referred to as CDD-95-126, ESP-305, CardioBiss® or Nephrobiss®) (EKR Therapeutics) a thirty-two amino acid cyclic peptide, and variants and derivatives thereof, which can be used in the particles, conjugates and compositions described herein to treat a kidney disorder, e.g., renal failure.

Kidney Disorders

The disclosed polymer-agent conjugates, particles and compositions are useful in treating kidney disorders, e.g., treating a kidney disorder described herein. In some embodiments, wherein the agent is a diagnostic agent, the polymer-agent conjugates, particles and compositions described herein can be used to evaluate or diagnose a kidney disorder.

Exemplary kidney disorders include, e.g., acute kidney failure, acute nephritic syndrome, analgesic nephropathy, atheroembolic renal disease, chronic kidney failure, chronic nephritis, congenital nephrotic syndrome, end-stage renal disease, goodpasture syndrome, interstitial nephritis, kidney damage, kidney infection, kidney injury, kidney stones, lupus nephritis, membranoproliferative GN I, membranoproliferative GN II, membranous nephropathy, minimal change disease, necrotizing glomerulonephritis, nephroblastoma, nephrocalcinosis, nephrogenic diabetes insipidus, nephrosis (nephrotic syndrome), polycystic kidney disease, post-streptococcal GN, reflux nephropathy, renal artery embolism, renal artery stenosis, renal papillary necrosis, renal tubular acidosis type I, renal tubular acidosis type II, renal underperfusion, and renal vein thrombosis.

In some embodiments, the agent is a derivative of a therapeutic peptide with pharmaceutical activity, such as an acetylated derivative or a pharmaceutically acceptable salt. In some embodiments, the therapeutic peptide is a prodrug such as a hexanoate conjugate.

Therapeutic peptide may mean a combination of therapeutic peptides that have been combined and attached to a polymer and/or loaded into the particle. Any combination of therapeutic peptides may be used. In certain embodiments for treating cancer, at least two traditional chemotherapeutic therapeutic peptides are attached to a polymer and/or loaded into the particle.

In certain embodiments, the therapeutic peptide may be attached to a polymer to form a therapeutic peptide-polymer conjugate.

In certain embodiments, the therapeutic peptide in the particle is attached to a polymer of the particle. The therapeutic peptide may be attached to any polymer in the particle, e.g., a hydrophobic polymer or a polymer containing a hydrophilic and a hydrophobic portion.

In certain embodiments, a therapeutic peptide is embedded in the particle. The therapeutic peptide may be associated with a polymer or other component of the particle through one or more non-covalent interactions such as van der Waals interactions, hydrophobic interactions, hydrogen bonding, dipole-dipole interactions, ionic interactions, and pi stacking.

A therapeutic peptide may be present in varying amounts of a therapeutic peptide-polymer conjugate, particle or composition described herein. When present in a particle, the therapeutic peptide may be present in an amount, e.g., from about 1 to about 100% by weight (e.g., from about 2 to about 30% by weight, from about 4 to about 25% by weight, from about 50 to about 100% by weight, from about 70 to about 100% by weight, from about 50 to about 90% by weight, or from about 5 to about 13%, 14%, 15%, 16%, 17%, 18%, 19% 20%, 30%, 40%, 50%, 60%. 70%, or 80% by weight).

Conjugates

One or more of the components of the particle can be in the form of a conjugate, i.e., attached to another moiety. Exemplary conjugates include therapeutic peptide/protein-polymer conjugates (e.g., a therapeutic peptide or protein-hydrophobic polymer conjugate, a therapeutic peptide or protein-hydrophobic-hydrophilic polymer conjugate, or a therapeutic peptide or protein-hydrophilic polymer conjugate), counterion-polymer conjugates (e.g., a counterion-hydrophobic polymer conjugate or a counterion-hydrophobic-hydrophilic polymer conjugate), and therapeutic peptide or protein-hydrophobic moiety conjugates.

A therapeutic peptide or protein-polymer conjugate described herein includes a polymer (e.g., a hydrophobic polymer, a hydrophilic polymer, or a hydrophilic-hydrophobic polymer) and a therapeutic peptide or protein. A therapeutic peptide or protein described herein may be attached to a polymer described herein, e.g., directly (e.g., without the presence of atoms from an intervening spacer moiety), or through a linker A therapeutic peptide or protein may be attached to a hydrophobic polymer (e.g., PLGA), a hydrophilic polymer (e.g., PEG) or a hydrophilic-hydrophobic polymer (e.g., PEG-PLGA). A therapeutic peptide or protein may be attached to a terminal end of a polymer, to both terminal ends of a polymer, or to a point along a polymer chain. In some embodiments, multiple therapeutic peptides or proteins may be attached to points along a polymer chain, or multiple therapeutic peptides or proteins may be attached to a terminal end of a polymer via a multifunctional linker. A therapeutic peptide or protein may be attached to a polymer described herein through the amino terminal or the carboxy terminal of the therapeutic peptide or protein. A therapeutic peptide or protein may also be attached to a polymer described herein through a functional group of a side chain of an amino acid that is part of the therapeutic peptide or protein.

A counterion-polymer conjugate described herein includes a polymer (e.g., a hydrophobic polymer or a polymer containing a hydrophilic portion and a hydrophobic portion) and a counterion. A counterion described herein may be attached to a polymer described herein, e.g., directly (e.g., without the presence of atoms from an intervening spacer moiety), or through a linker. A counterion may be attached to a hydrophobic polymer (e.g., PLGA) or a polymer having a hydrophobic portion and a hydrophilic portion (e.g., PEG-PLGA). A counterion may be attached to a terminal end of a polymer, to both terminal ends of a polymer, or to a point along a polymer chain. In some embodiments, multiple counterions may be attached to points along a polymer chain, or multiple counterions may be attached to a terminal end of a polymer via a multifunctional linker

Modes of Attachment

A therapeutic peptide, protein or counterion described herein may be directly (e.g., without the presence of atoms from an intervening spacer moiety), attached to a polymer or hydrophobic moiety described herein (e.g., a polymer). The attachment may be at a terminus of the polymer or along the backbone of the polymer. In some embodiments, the therapeutic peptide or protein is modified at the point of attachment to the polymer; for example, a terminal amine or a terminal carboxylic acid moiety of the therapeutic peptide or protein is converted to a functional group that is reacted with the polymer (e.g., the carboxylic acid moiety is converted to a thioester moiety). A reactive functional group of a therapeutic peptide, protein or counterion may be directly attached (e.g., without the presence of atoms from an intervening spacer moiety), to a functional group on a polymer. A therapeutic peptide, protein or counterion may be attached to a polymer via a variety of linkages, e.g., an amide, ester, sulfide (e.g., a maleimide sulfide), disulfide, succinimide, oxime, silyl ether, carbonate or carbamate linkage. For example, in one embodiment, a carboxylic group of a therapeutic peptide, protein or counterion may be reacted with a hydroxy group of a polymer, forming a direct ester linkage between the therapeutic peptide, protein or counterion and the polymer. In another embodiment, an amine group of a therapeutic peptide, protein or counterion may be linked to a carboxylic acid group of a polymer, forming an amide bond. In an embodiment a thiol modified therapeutic peptide or protein may be reacted with a reactive moiety on the terminal end of the polymer (e.g., an acrylate PLGA, or a pyridinyl-SS-activated PLGA, or a maleimide activated PLGA) to form a sulfide or disulfide or thioether bond (i.e., sulfide bond). Exemplary modes of attachment include those resulting from click chemistry (e.g., an amide bond, an ester bond, a ketal, a succinate, or a triazole and those described in WO 2006/115547).

In certain embodiments, suitable protecting groups may be required on the other polymer terminus or on reactive side chains of the therapeutic peptide or protein, to facilitate formation of the specific desired conjugate. For example, a polymer having a hydroxy terminus may be protected, e.g., with a silyl group group (e.g., trimethylsilyl) or an acyl group (e.g., acetyl). A therapeutic peptide or protein having one or more reactive groups on a side chain may be protected, e.g., with an acetyl group, on a hydroxyl or amino group, such that the therapeutic peptide or protein may be selectively attached to a polymer, e.g., through the terminal end of the therapeutic peptide or protein.

In some embodiments, the process of attaching a therapeutic peptide, protein or counterion to a polymer may result in a composition comprising a mixture of conjugates having the same polymer and the same therapeutic peptides, proteins or counterions, but which differ in the nature of the linkage between the therapeutic peptide, protein or counterion and the polymer. For example, when a therapeutic peptide, protein or counterion has a plurality of reactive moieties that may react with a polymer, the product of a reaction of the therapeutic peptide, protein or counterion and the polymer may include a conjugate wherein the therapeutic peptide, protein or counterion is attached to the polymer via one reactive moiety, and a conjugate wherein the therapeutic peptide, protein or counterion is attached to the polymer via another reactive moiety. For example, when a therapeutic peptide or protein is attached to a polymer, the product of the reaction may include a conjugate where some of the therapeutic peptide or protein is attached to the polymer through the carboxy terminal of the therapeutic peptide or protein and some of the therapeutic peptide or protein is attached to the polymer through the amino terminal of the therapeutic peptide or protein. Likewise, where a counterion has multiple reactive groups such as a plurality of amines, the product of the reaction may include a conjugate where some of the counterion is attached to the polymer through a first reactive group and some of the counterion is attached to the polymer through a second reactive group.

In some embodiments, the process of attaching a therapeutic peptide, protein or counterion to a polymer may involve the use of protecting groups. For example, when a therapeutic peptide, protein or counterion has a plurality of reactive moieties that may react with a polymer, the therapeutic peptide, protein or counterion may be protected at certain reactive positions such that a polymer will be attached via a specified position. In one embodiment, the therapeutic peptide or protein may be protected on the carboxy terminal or the amino terminal of the therapeutic peptide or protein when attaching to a polymer. In one embodiment, a therapeutic peptide or protein may be protected on a side chain of the therapeutic peptide or protein when attaching to a polymer. In one embodiment, a therapeutic peptide or protein may be protected on a side chain and a terminal end (e.g., an amino terminal or a carboxy terminal) of the therapeutic peptide or protein.

In some embodiments, selectively-coupled products such as those described above may be combined to form mixtures of therapeutic peptide/protein-polymer conjugates. For example, PLGA attached to a therapeutic peptide or protein through the carboxy terminal of the therapeutic peptide or protein, and PLGA attached to a therapeutic peptide or protein through the amino terminal of the therapeutic peptide or protein, may be combined to form a mixture of the two conjugates, and the mixture may be used in the preparation of a particle.

A polymer-agent (e.g., a polymer-therapeutic peptide or polymer-protein) conjugate may comprise a single therapeutic peptide or protein or counterion attached to a polymer. The therapeutic peptide, protein or counterion may be attached to a terminal end of a polymer, or to a point along a polymer chain.

In some embodiments, the conjugate may comprise a plurality of therapeutic peptides, proteins or counterions attached to a polymer (e.g., 2, 3, 4, 5, 6 or more agents may be attached to a polymer). The therapeutic peptides, proteins or counterions may be the same or different. In some embodiments, a plurality of therapeutic peptides, proteins or counterions may be attached to a multifunctional linker (e.g., a polyglutamic acid linker). In some embodiments, a plurality of therapeutic peptides, proteins or counterions may be attached to points along the polymer chain.

Linkers

A therapeutic peptide, protein or counterion may be attached to a moiety such as a polymer or a hydrophobic moiety such as a lipid, or to each other, via a linker, such as a linker described herein. For example: a hydrophobic polymer may be attached to a counterion; a hydrophobic polymer may be attached to a therapeutic peptide or protein; a hydrophilic-hydrophobic polymer may be attached to a therapeutic peptide or protein; a hydrophilic polymer may be attached to a therapeutic peptide or protein; a hydrophilic polymer may be attached to a counterion; or a hydrophobic moiety may be attached to a counterion, or a therapeutic peptide or protein may be attached to a counterion. A therapeutic peptide or protein may be attached to a moiety such as a polymer described herein through the carboxylic acid position of the therapeutic peptide or protein, such as a terminal carboxylic acid position of the therapeutic peptide or protein (e.g., through a linker described herein). A therapeutic peptide or rpotein may be attached to a moiety such as a polymer described herein through the amine position of the therapeutic peptide or protein, such as a terminal amine position of the therapeutic peptide or protein (e.g., through a linker described herein). In some embodiments, the therapeutic peptide or protein is attached through a terminal end of a polymer (e.g., a PLGA polymer, where the attachment is at the hydroxyl terminal or carboxy terminal).

In certain embodiments, a plurality of the linker moieties is attached to a polymer, allowing attachment of a plurality of therapeutic peptides, proteins or counterions to the polymer through linkers, for example, where the linkers are attached at multiple places on the polymer such as along the polymer backbone. In some embodiments, a linker is configured to allow for a plurality of a first moiety to be linked to a second moiety through the linker, for example, a plurality of therapeutic peptides or proteins can be linked to a single polymer such as a PLGA polymer via a branched linker, wherein the branched linker comprises a plurality of functional groups through which the therapeutic peptides or proteins can be attached. In some embodiments, the therapeutic peptide or protein is released from the linker under biological conditions (i.e., cleavable under physiological conditions). In another embodiment a single linker is attached to a polymer, e.g., at a terminus of the polymer.

The linker may comprise, for example, an alkylene (divalent alkyl) group. In some embodiments, one or more carbon atoms of the alkylene linker may be replaced with one or more heteroatoms or functional groups (e.g., thioether, amino, ether, keto, amide, silyl ether, oxime, carbamate, carbonate, disulfide, or heterocyclic or heteroaromatic moieties). For example, an acrylate polymer (e.g., an acrylate PLGA) can be reacted with a thiol modified therapeutic peptide or protein to form a therapeutic peptide/protein-polymer conjugate attached through a sulfide bond. The acrylate can be attached to a terminal end of the polymer (e.g., a hydroxyl terminal end of a PLGA polymer such as a 50:50 PLGA polymer) by reacting an acrylacyl chloride with the hydroxyl terminal end of the polymer.

In some embodiments, a linker, in addition to the functional groups that allow for attachment of a first moiety to a second moiety, has an additional functional group. In some embodiments, the additional functional group can be cleaved under physiological conditions. Such a linker can be formed, for example, by reacting a first activated moiety such as a therapeutic peptide or protein, e.g., a therapeutic peptide or protein described herein, with a second activated moiety such as a polymer, e.g., a polymer described herein, to produce a linker that includes a functional group that is formed by joining the therapeutic peptide or protein to the polymer. Optionally, the additional functional group can provide a site for additional attachments or allow for cleavage under physiological conditions. For example, the additional functional group may include a sulfide, disulfide, ester, oxime, carbonate, carbamate, or amide bonds that are cleavable under physiological conditions. In some embodiments, one or both of the functional groups that attach the linker to the first or second moiety may be cleavable under physiological conditions such as esters, amides, or disulfides.

In some embodiments, the additional functional group is a heterocyclic or heteroaromatic moiety.

A therapeutic peptide or protein may be attached through a linker (e.g., a linker comprising two or three functional groups such as a linker described herein) to a moiety such as a polymer described herein through a carboxylic acid or amine group of the therapeutic peptide or protein, such as a terminal carboxylic acid or amine of the therapeutic peptide or protein, or through a reactive group on a side chain of an amino acid of the therapeutic peptide or protein. In some embodiments, the therapeutic peptide or protein is attached through a terminal end of a polymer (e.g., a PLGA polymer, where the attachment is at the hydroxyl terminal or carboxy terminal).

In some embodiments, the linker includes a moiety that can modulate the reactivity of a functional group in the linker (e.g., another functional group or atom that can increase or decrease the reactivity of a functional group, for example, under biological conditions).

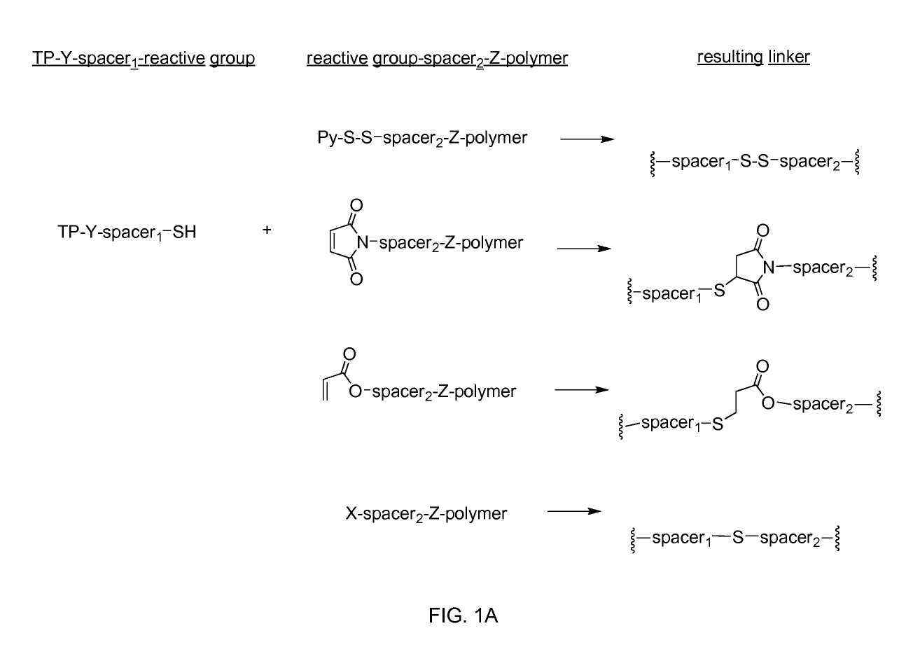

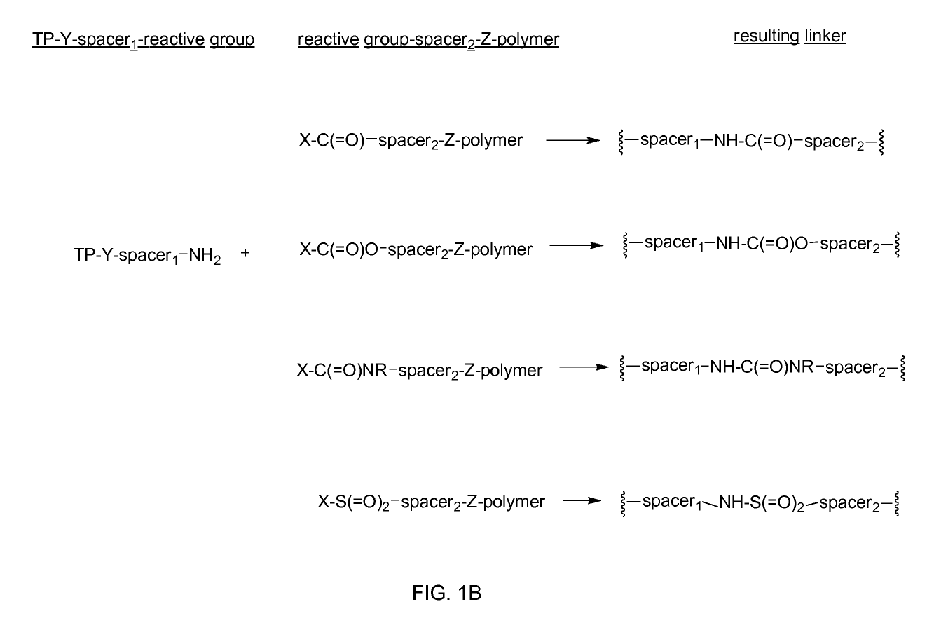

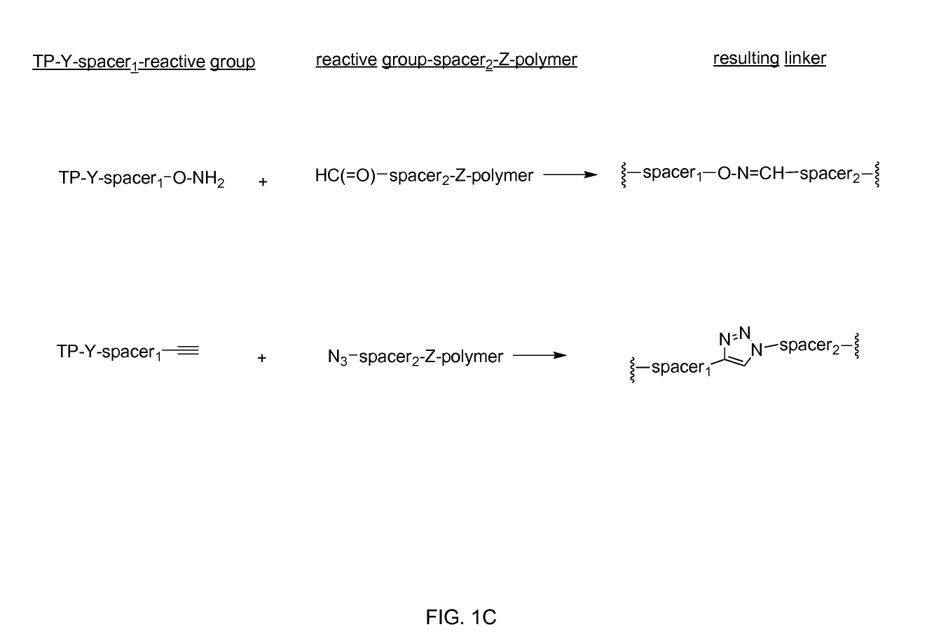

For example, as shown in FIGS. 1A-C, a therapeutic peptide (TP), having a first reactive group may be reacted with a polymer having a second reactive group to attach the therapeutic peptide to the polymer while providing a biocleavable functional group. The resulting linker includes a first spacer such as an alkylene spacer that attaches the therapeutic peptide to the functional group resulting from the attachment (i.e., by way of formation of a covalent bond), and a second spacer such as an alkylene spacer (e.g., from about C₁ to about C₆) that attaches the polymer to the functional group resulting from the attachment.

As shown in FIGS. 1A-C, the therapeutic peptide may be attached to the first spacer via a moiety Y, which may also be biocleavable. Y may be, for example, —O—, —S—, —NH—, —C(═O)NH—, or —C(═O)O—. In some embodiments, the second spacer may be attached to a leaving group X—, for example halo (e.g., chloro) or N-hydroxysuccinimidyl (NHS). The second spacer may be attached to the polymer via an additional functional group (Z) that links with the polymer terminus, e.g., a terminal —OH, —CO₂H, —NH₂, or —SH, of a polymer, e.g., a terminal —OH or —CO₂H of PLGA. The additional functional group (Z) may be, for example, —O—, —OC(═O)—, —OC(═O)O—, —OC(═O)NR—, —NR—, —NRC(═O)—, —NRC(═O)O—, —NRC(═O)NR′—, —NRS(═O)₂—, —S—, —S(═O)—, —S(═O)₂—, —C(═O)O—, or —C(═O)NR—, and provides an additional site for reactivity, e.g., attachment or cleavage. The therapeutic peptide may be attached through a carboxylic acid or amine group of the therapeutic peptide, such as a terminal carboxylic acid or amine of the therapeutic peptide, or through a reactive group on a side chain of an amino acid of the therapeutic peptide. In some embodiments, the therapeutic peptide is attached through a spacer to the terminal end of a polymer (e.g., a PLGA polymer, where the attachment is at the hydroxyl terminal or carboxy terminal).

In an embodiment, e.g., as shown in FIG. 1A, a thiol modified therapeutic peptide can be reacted with a pyridynyl-SS-activated polymer (e.g., a pyridynyl-SS-activated PLGA, e.g., pyridynyl-SS-activated 5050 PLGA) to form a therapeutic peptide-polymer conjugate attached through a disulfide bond. In an embodiment, a thiol modified therapeutic peptide can be reacted with a maleimide-activated polymer (e.g., a maleimide-activated PLGA, e.g., maleimide-activated 5050 PLGA) to form a therapeutic peptide-polymer conjugate attached through a maleimide sulfide bond. In an embodiment, a thiol modified therapeutic peptide can be reacted with an acrylate-activated polymer (e.g., an acrylate-activated PLGA, e.g., acrylate-activated 5050 PLGA) to form a therapeutic peptide-polymer conjugate through a mercaptoproponate bond. The therapeutic peptide may be attached through a carboxylic acid or amine group of the therapeutic peptide, such as a terminal carboxylic acid or amine of the therapeutic peptide, or through a reactive group on a side chain of an amino acid of the therapeutic peptide. In some embodiments, the therapeutic peptide is attached through a spacer to the terminal end of a polymer (e.g., a PLGA polymer, where the attachment is at the hydroxyl terminal or carboxy terminal).

In an embodiment, e.g., as shown in FIG. 1B, an amine modified therapeutic peptide can be reacted with an polymer having an activated carboxylic acid or ester (e.g., an activated carboxylic acid PLGA, e.g., activated carboxylic acid 5050 PLGA, e.g., an SPA activated carboxylic acid PLGA, e.g., an SPA activated carboxylic acid5050 PLGA) to form a therapeutic peptide-polymer conjugate attached through an amide bond. In an embodiment, an amine modified therapeutic peptide can be reacted with an activated polymer (e.g., an activated PLGA, e.g.,—activated 5050 PLGA) to form a therapeutic peptide-polymer conjugate attached through a carbamate bond. In an embodiment, an amine modified therapeutic peptide can be reacted with an activated polymer (e.g., an activated PLGA, e.g., activated 5050 PLGA) to form a therapeutic peptide-polymer conjugate attached through a carbamide bond (urea). In an embodiment, an amine modified therapeutic peptide can be reacted with an activated polymer (e.g., an activated PLGA, e.g., activated 5050 PLGA,) to form a therapeutic peptide-polymer conjugate attached through an aminoalkylsulfonamide bond. The therapeutic peptide may be attached through a carboxylic acid or amine group of the therapeutic peptide, such as a terminal carboxylic acid or amine of the therapeutic peptide, or through a reactive group on a side chain of an amino acid of the therapeutic peptide. In some embodiments, the therapeutic peptide is attached through a spacer to the terminal end of a polymer (e.g., a PLGA polymer, where the attachment is at the hydroxyl terminal or carboxy terminal).

In an embodiment, e.g., as shown in FIG. 1C, a hydroxylamine modified therapeutic peptide can be reacted with an aldehyde-activated polymer (e.g., an aldehyde-activated PLGA, e.g., aldehyde-activated 5050 PLGA, e.g., a formaldehyde-activated PLGA, e.g., formaldehyde-activated 5050 PLGA) to form a therapeutic peptide-polymer conjugate attached through an aldoxime bond. The therapeutic peptide may be attached through a carboxylic acid or amine group of the therapeutic peptide, such as a terminal carboxylic acid or amine of the therapeutic peptide, or through a reactive group on a side chain of an amino acid of the therapeutic peptide. In some embodiments, the therapeutic peptide is attached through a spacer to the terminal end of a polymer (e.g., a PLGA polymer, where the attachment is at the hydroxyl terminal or carboxy terminal).

In an embodiment, e.g., as shown in FIG. 1C, an alkylyne modified therapeutic peptide can be reacted with an azide-activated polymer (e.g., an azide-activated PLGA, e.g., azide-activated 5050 PLGA) to form a therapeutic peptide-polymer conjugate attached through a triazole bond. The therapeutic peptide may be attached through a carboxylic acid or amine group of the therapeutic peptide, such as a terminal carboxylic acid or amine of the therapeutic peptide, or through a reactive group on a side chain of an amino acid of the therapeutic peptide. In some embodiments, the therapeutic peptide is attached through a spacer to the terminal end of a polymer (e.g., a PLGA polymer, where the attachment is at the hydroxyl terminal or carboxy terminal).

In some embodiments, the linker, prior to attachment to the agent and the polymer, may have one or more of the following functional groups: amine, amide, hydroxyl, carboxylic acid, ester, halogen, thiol, maleimide, carbonate, or carbamate. In some embodiments, the functional group remains in the linker subsequent to the attachment of the first and second moiety through the linker. In some embodiments, the linker includes one or more atoms or groups that modulate the reactivity of the functional group (e.g., such that the functional group cleaves such as by hydrolysis or reduction under physiological conditions).

In some embodiments, the linker may comprise an amino acid or a peptide within the linker Frequently, in such embodiments, the peptide linker is cleavable by hydrolysis, under reducing conditions, or by a specific enzyme (e.g., under physiological conditions).

When the linker is the residue of a divalent organic molecule, the cleavage of the linker may be either within the linker itself, or it may be at one of the bonds that couples the linker to the remainder of the conjugate, e.g., either to the therapeutic peptide or the polymer.

In some embodiments, a linker may be selected from one of the following or a linker may comprise one of the following:

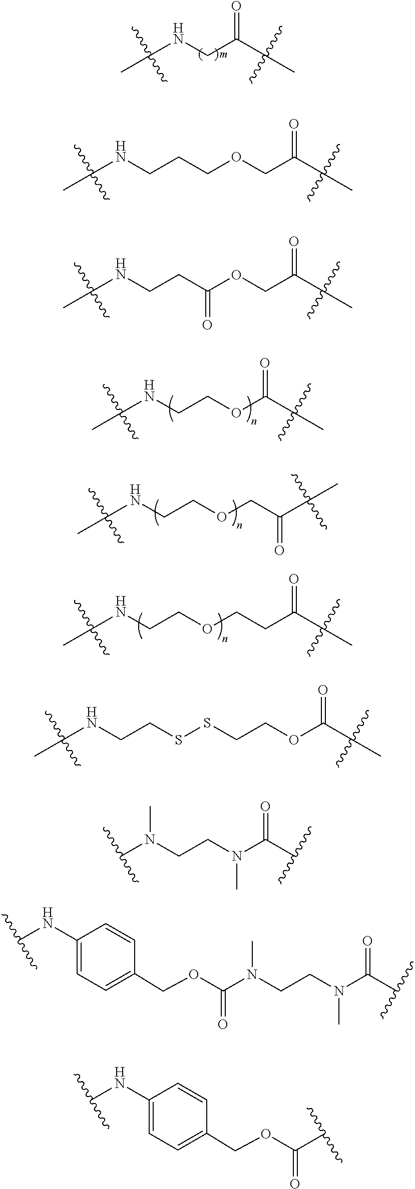

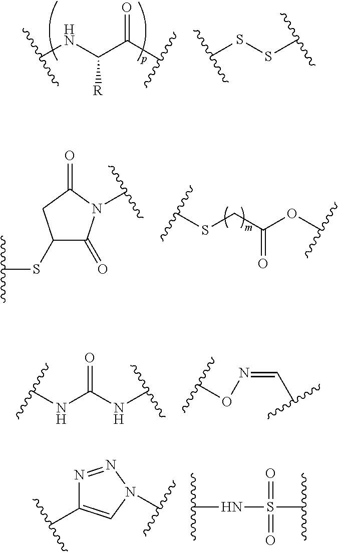

wherein m is 1-10, n is 1-10, p is 1-10, and R is an amino acid side chain.

A linker may include a bond resulting from click chemistry (e.g., an amide bond, an ester bond, a ketal, a succinate, or a triazole and those described in WO 2006/115547). A linker may be, for example, cleaved by hydrolysis, reduction reactions, oxidative reactions, pH shifts, photolysis, or combinations thereof; or by an enzyme reaction. The linker may also comprise a bond that is cleavable under oxidative or reducing conditions, or may be sensitive to acids.

In some embodiments, the linker is not cleaved under physiological conditions, for example, the linker is of a sufficient length that the therapeutic peptide does not need to be cleaved to be active, e.g., the length of the linker is at least about 20 angstroms (e.g., at least about 30 angstroms or at least about 50 angstroms).

Methods of Making Therapeutic Peptide-Polymer Conjugates and Protein-Polymer Conjugates

The therapeutic peptide-polymer conjugates and protein-polymer conjugates may be prepared using a variety of methods known in the art, including those described herein. In some embodiments, to covalently link the agent to a polymer, the polymer or agent may be chemically activated using any technique known in the art. The activated polymer is then mixed with the agent, or the activated agent is mixed with the polymer, under suitable conditions to allow a covalent bond to form between the polymer and the agent. In some embodiments, a nucleophile, such as a thiol, hydroxyl group, or amino group, on the agent attacks an electrophile (e.g., activated carbonyl group) to create a covalent bond. An agent may be attached to a polymer via a variety of linkages, e.g., an amide, ester, succinimide, carbonate or carbamate linkage.

The coupling reactions generally occur in a solvent system, and can include a mixture of solvents. Exemplary water miscible solvents include acetone, DMSO, aceotnitrile, DMF, dioxane, and THF. Exemplary water immiscible solvents include ethyl acetate, benzyl alcohol, chloroform, and dichloromethane. The solvent systems can vary based on the length and types of amino acids present in the peptide or protein. In some embodiments, an aqueous buffer solution can be used, for example, with a hydrophilic peptide. In some embodiments, minimal amounts or none of the following solvents are used: acetic acid, aceonitrile, DMF, DMSO, ethanol, or isopropyl alcohol.

In some embodiments, an agent may be attached to a polymer via a linker. In such embodiments, a linker may be first covalently attached to a polymer, and then attached to an agent. In other embodiments, a linker may be first attached to an agent, and then attached to a polymer.

Exemplary Therapeutic Peptide-Polymer Conjugates Therapeutic peptide-polymer conjugates can be made using many different combinations of components described herein. For example, various combinations of polymers (e.g., PLGA, PLA or PGA), linkers attaching the therapeutic peptide to the polymer, and therapeutic peptides are described herein.

Exemplary therapeutic peptide-polymer conjugates include the following.

1) PLGA-Ester Linker-Therapeutic Peptide

This conjugate will generally include the modification of carbonyl end group of peptide with amino group which can be conjugated to the PLGA polymer. This linker will have an ester bond to the therapeutic peptide which can be cleaved off at high pH or by an enzyme such as estearase. An exemplary scheme is shown below.

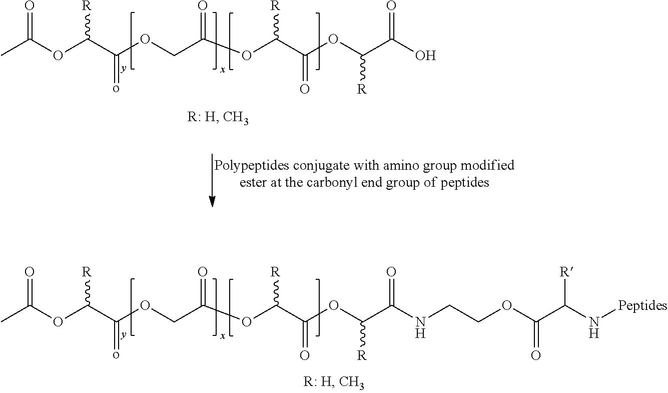

2) PLGA-Amide Linker-Therapeutic Peptide

This conjugate will generally include the modification of carbonyl end group of PLGA with an amine functional group. The amino group of PLGA derivatives then can react with carbonyl end group of therapeutic peptide or carbonyl groups on the side chains of amino acids such as glutamic acid or aspartic acid to form a stable amide bond. An exemplary scheme is shown below.

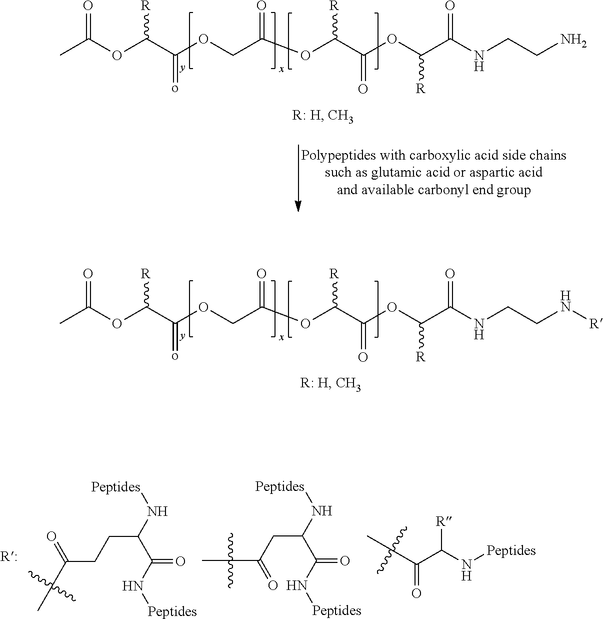

3) PLGA-Disulfide Linker-Therapeutic Peptide

This conjugate will generally include the modification of carbonyl end group of PLGA with a reactive sulfihydryl group. This group can react with therapeutic peptides containing cysteine groups which could be located at the end group or along the chain. It can also react with peptides that are derivatized with sulfihydryl group. The disulfide bond can be reduced internally to release peptide. An exemplary scheme is shown below.

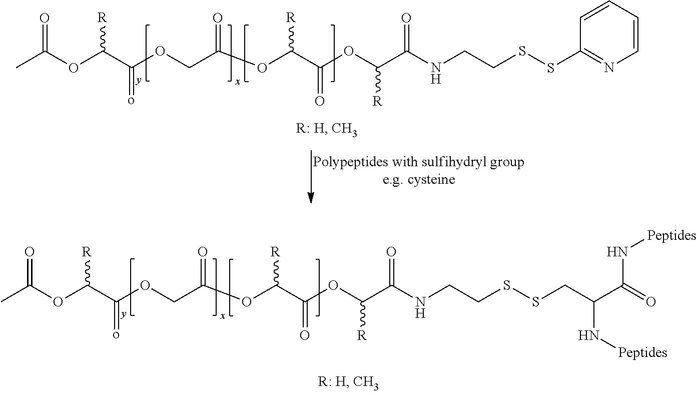

4) PLGA-Disulfide Linker-Therapeutic Peptide

This conjugate will generally include the modification of hydroxyl group on tyrosine with disulfide amino group which can be conjugated to PLGA. Upon reduction of disulfide bond, the linker will cyclize and kick out the polypeptides. Tyrosine or phenol group derivatized amino acids can be used. The disulfide bond can be reduced internally to release therapeutic peptide. An exemplary scheme is shown below.

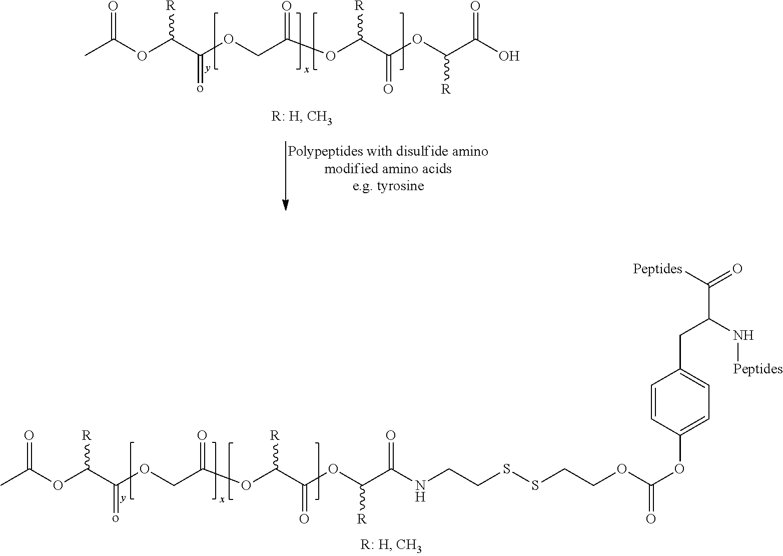

5) PLGA-Thioether Linker-Therapeutic Peptide

This conjugate will generally include the modification of the carbonyl end group of PLGA with a maleimide group. This group can react with therapeutic peptides containing cysteine located at the end group or along the peptide chain. It can also react with peptides that are derivatized with sulfihydryl group. This conjugate will have a non-releasing thioether bond. An exemplary scheme is shown below.

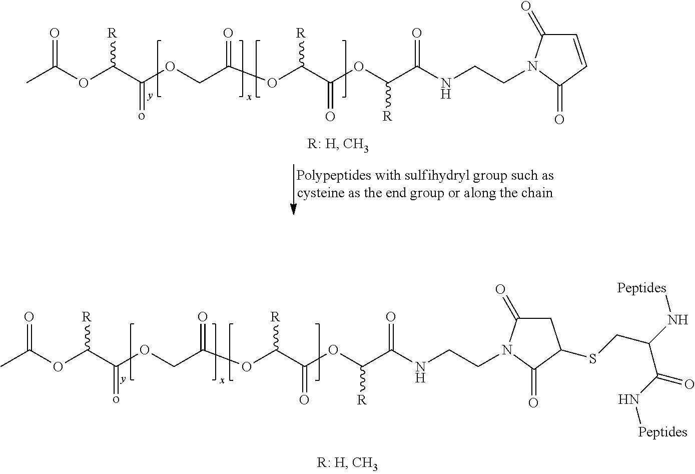

6) Alkyne Terminated PLGA/Azide Functional Therapeutic Peptide

A PLGA polymer terminated with an acetylene group (i.e., alkyne) can be conjugated to a therapeutic peptide. A terminal amino-functional group (e.g., glycine) can be converted to an alkyen moiety via a coupling reaction with 4-pentynoic acid in the presence of N,N′-dicyclohexylcarbodiimide. The reaction can also be done using click chemistr, for example, using a catalyst such as copper bromide to react an azide terminated polymer (e.g., an azide terminated PLGA polymer) and an alkyne functional therapeutic peptide. 2,2′-bipyridyl can also be dissolved in N-methyl pyrrolidone to complex copper bromide and 2,2′-bipyridyl, which could be dialyzed against water (e.g., pure water). The reaction can be performed on a solid support, e.g, to prepare an azide functionalized therapeutic peptide. An exemplary reaction scheme is shown below.

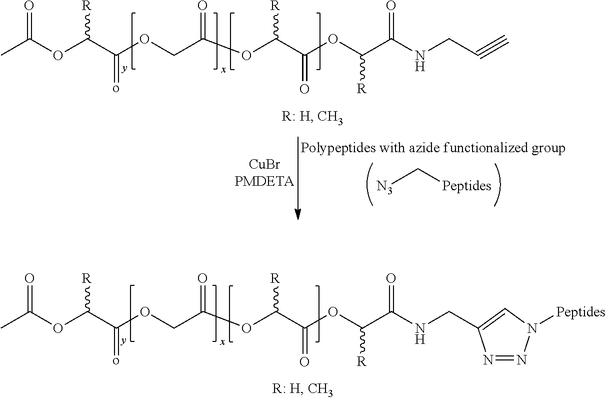

7) Linker Formed by Diels Alder Chemistry

A PLGA polymer terminated with a moiety that can be used in a reaction of a conjugated diene to an alkene group to form a cyclohexene group, linking the therapeutic peptide to the polymer. Exemplary Diels Alder reactions can be done using a Michael's Addition (1,4 addition), for example, in the presense of a base (NaOH or KOH) to form an enolate. The resulting enolate can then reacts with α,β-unsaturated ketones. Additional exemplary reactions include an epoxy ring opening, for example, with amine or hydroxyl groups (nucelophilic substitution-Sn2 reaction).

8) Linkers Used in Antibody Drug Conjugates

Exemplary linkers include acid labile hydrazone linkers: (6-maleimidocaproyl) hydrazone linker to cysteine residues (e.g., as used in BR96-doxorubicin, BMS); and 4-(4′-acetylphenoxy)butanoic acid (e.g., as used in Mylotarg, Pfizer).

Additional linkers include enzyme linked conjugates. Certain advantages to such linkers include improved stability in blood circulation relative to hydrazone linkers. Exemplary enzyme linked conjugates include Valine-citrulline, Valine-lysine (Seattle Genetics), and Phenylalanine-lysine.

9) Linkers Synthesized Using Click Chemistry

A PLGA polymer terminated with an alkyne group (e.g. acetylene) can be conjugated to a therapeutic peptide with an azide group, or a PLGA polymer terminated with an azide group can be conjugated to a therapeutic peptide with an alkyne group. In order to be able to release the therapeutic peptide more easily, a cleavable linker (e.g. ester or disulfide) can be introduced in between the azide or alkyne functional group and the therapeutic peptide.

A PLGA terminated with an acetylene group (alkyne) can be reacted, with an azide functional therapeutic peptide. The synthesis can include the use of an insoluble substrate, e.g., to functionalize the therapeutic peptide. In some embodiments, a terminal amino-functional group (e.g. glycine) can be converted into an alkyne moiety via a coupling reaction with 4-pentynoic acid in the presence of N,N′-dicyclohexylcarbodiimide

Other exemplary coupling reactions using click chemistry include a Michael Addition (1,4 addition) (e.g., addition of a base (NaOH or KOH) to form an enolate, and allowing the enolate to react with an α,β-unsaturated ketone); Diels Alder reaction (e.g., reaction of a conjugated diene to an alkene group to form a cyclohexene group); and an epoxy ring opening with amine or hydroxyl groups (e.g., a nucelophilic substitution-Sn2 reaction).

### Compositions of Therapeutic Peptide-Polymer Conjugates and Protein-Polymer Conjugates

Compositions of therapeutic peptide/protein-polymer conjugates described above may include mixtures of products. For example, the conjugation of a therapeutic peptide or protein to a polymer may proceed in less than 100% yield, and the composition comprising the therapeutic peptide/protein-polymer conjugate may thus also include unconjugated polymer.

Compositions of therapeutic peptide/protein-polymer conjugates may also include therapeutic peptide/protein-polymer conjugates that have the same polymer and the same agent, and differ in the nature of the linkage between the agent and the polymer. The therapeutic peptide/protein-polymer conjugates may be present in the composition in varying amounts. For example, when a therapeutic peptide or protein having a plurality of available attachment points is reacted with a polymer, the resulting composition may include more of a product conjugated via a more reactive carboxyl group, and less of a product attached via a less reactive carboxyl group.

Additionally, compositions of therapeutic peptide/protein-polymer conjugates may include therapeutic peptides or proteins that are attached to more than one polymer chain.

### Surfactants

In some embodiments, a particle described herein comprises a surfactant. Exemplary surfactants include PEG, poly(vinyl alcohol) (PVA), poly(vinylpyrrolidone) (PVP), poloxamer, a polysorbate, a polyoxyethylene ester, a PEG-lipid (e.g., PEG-ceramide, d-alpha-tocopheryl polyethylene glycol 1000 succinate), 1,2-distearoyl-sn-glycero-3-phosphoehanolamine or lecithin. In some embodiments, the surfactant is PVA and the PVA is from about 3 kDa to about 50 kDa (e.g., from about 5 kDa to about 45 kDa, about 7 kDa to about 42 kDa, from about 9 kDa to about 30 kDa, or from about 11 to about 28 kDa) and up to about 98% hydrolyzed (e.g., about 75-95%, about 80-90% hydrolyzed, or about 85% hydrolyzed). In some embodiments, the surfactant is polysorbate 80. In some embodiments, the surfactant is SOLUTOL® HS 15 (BASF, Florham Park, N.J.). In some embodiments, the surfactant is present in an amount of up to about 35% by weight of the system (e.g., up to about 20% by weight or up to about 25% by weight, from about 15% to about 35% by weight, from about 20% to about 30% by weight, or from about 23% to about 26% by weight).

Counterions

A particle described herein may also include one or more counterions, e.g., a charged moiety, a cationic moiety, an anionic moiety, or a zwitterionic moiety. The counterion may neutralize a charge associated with a therapeutic peptide or protein thereby allowing for improved formulations (e.g., improved stability, solubility, or transport). In some embodiments, the charged moiety is associated with a therapeutic peptide or protein (e.g., hydrogen bonded to the therapeutic peptide or protein, or part of a solvation layer around the therapeutic peptide or protein). In some embodiments, the charged moiety is covalently attached to a polymer of a particle described herein. In some embodiments, the charged moiety is covalently attached to a polymer that is covalently attached to a therapeutic peptide or protein. In some embodiments the charged moiety is a peptide.

In some embodiments, a charged moiety is covalently attached to a hydrophobic polymer via a linker (e.g., at the carboxy terminal or hydroxyl terminal of the hydrophobic polymers). In some embodiments, the linker comprises a bond formed using click chemistry (e.g., as described in WO 2006/115547). In some embodiments, the linker comprises an amide bond, an ester bond, a disulfide bond, a sulfide bond, a ketal, a succinate, or a triazole. In some embodiments, a single charged moiety is covalently attached to a single hydrophobic polymer (e.g., at the terminal end of the hydrophobic polymer). In some embodiments, a charged moiety is covalently attached to a hydrophilic-hydrophobic polymer through the hydrophobic portion via an amide, ester or ether bond. In some embodiments, a single hydrophobic polymer is covalently attached to a plurality of charged moieties. In some embodiments, at least a portion of the plurality of charged moieties are attached to the backbone of at least a portion of the hydrophobic polymers.

In some embodiments, a cationic moiety is a cationic polymer (e.g., PEI, cationic PVA, poly(histidine), poly(lysine), or poly(2-dmethylamino)ethyl methacrylate). In some embodiments, a cationic moiety is an amine (e.g., a primary, secondary, tertiary or quaternary amine). In some embodiments, at least a portion of the cationic moieties comprise a plurality of amines (e.g., a primary, secondary, tertiary or quaternary amines). In some embodiments, at least one amine in the cationic moiety is a secondary or tertiary amine. In some embodiments, at least a portion of the cationic moieties comprise a polymer, for example, polyethylene imine or polylysine Polymeric cationic moieties have a variety of molecular weights (e.g., ranging from about 500 to about 5000 Da, for example, from about 1 to about 2 kDa or about 2.5 kDa).

In some embodiments the cationic moiety is a polymer, for example, having one or more secondary or tertiary amines, for example cationic PVA (e.g., as provided by Kuraray, such as CM-318 or C-506), chitosan, and polyethyleneamine. Cationic PVA can be made, for example, by polymerizing a vinyl acetate/N-vinaylformamide co-polymer, e.g., as described in US 2002/0189774, the contents of which are incorporated herein by reference. Other examples of cationic PVA include those described in U.S. Pat. No. 6,368,456 and Fatehi (Carbohydrate Polymers 79 (2010) 423-428, the contents of which are incorporated herein by reference. In some embodiments, at least a portion of the cationic moieties of comprise a cationic PVA (e.g., as provided by Kuraray, such as CM-318 or C-506).

Other exemplary cationic moieties include poly(histidine) and poly(2-dmethylamino)ethyl methacrylate). In some embodiments, the amine is positively charged at acidic pH. In some embodiments, the amine is positively charged at physiological pH. In some embodiments, at least a portion of the cationic moieties are selected from the group consisting of protamine sulfate, hexademethrine bromide, cetyl trimethylammonium bromide, spermine, and spermidine. In some embodiments, at least a portion of the cationic moieties are selected from the group consisting of tetraalkyl ammonium moieties, trialkyl ammonium moieties, imidazolium moieties, aryl ammonium moieties, iminium moieties, amidinium moieties, guanadinium moieties, thiazolium moieties, pyrazolylium moieties, pyrazinium moieties, pyridinium moieties, and phosphonium moieties. In some embodiments, at least a portion of the cationic moieties are cationic lipids. In some embodiments, at least a portion of the cationic moieties are conjugated to a non-polymeric hydrophobic moiety (e.g., cholesterol or Vitamin E TPGS). In some embodiments, the plurality of cationic moieties are from about 1 to about 60 weight % of the particle. In some embodiments, the ratio of the charge of the plurality of cationic moieties to the charge from the plurality of therapeutic peptides is from about 1:1 to about 50:1 (e.g., 1:1 to about 10:1 or 1:1 to 5:1).

Exemplary cationic moieties for use in the particles and conjugates described herein include amines such as polyamines (e.g., polyethyleneimine (PEI) or derivatives thereof such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives), cationic lipids (e.g., DOTIM, dimethyldioctadecyl ammonium bromide, 1,2 dioleyloxypropyl-3-trimethyl ammonium bromide, DOTAP, 1,2-dimyristyloxypropyl-3-dimethyl-hydroxyethyl ammonium bromide, EDMPC, ethyl-PC, DODAP, DC-cholesterol, and MBOP, CLinDMA, pCLinDMA, eCLinDMA, DMOBA, and DMLBA), polyamino acids (e.g., poly(lysine), poly(histidine), and poly(arginine)) and polyvinyl pyrrolidone (PVP). The cationic moiety can be positively charged at physiological pH.

Additional exemplary cationic moieties include protamine sulfate, hexademethrine bromide, cetyl trimethylammonium bromide, spermine, spermidine, and those described for example in WO2005007854, U.S. Pat. No. 7,641,915, and WO2009055445, the contents of each of which are incorporated herein by reference. Cationic moieties may include N-methyl D-glucamine, choline, arginine, lysine, procaine, tromethamine (TRIS), spermine, N-methyl-morpholine, glucosamine, N,N-bis 2-hydroxyethyl glycine, diazabicycloundecene, creatine, arginine ethyl ester, amantadine, rimantadine, ornithine, taurine, and citrulline. Cationic moieties may additionally include sodium, potassium, calcium, magnesium, ammonium, monoethanolamine, diethanolamine, triethanolamine, tromethamine, lysine, histidine, arginine, morpholine, methylglucamine, and glucosamine.

Anionic moieties which may be suitable for formulation with net positively charged therapeutic peptides or proteins include, but are not limited to, acetate, propionate, butyrate, pentanoate, hexanoate, heptanoate, levulinate, chloride, bromide, iodide, citrate, succinate, maleate, glycolate gluconate, glucuronate, 3-hydroxyisobutyrate, 2-hydroxyisobutyrate, lactate, malate, pyruvate, fumarate, tartarate, tartronate, nitrate, phosphate, benzene sulfonate, methane sulfonate, sulfate, sulfonate, acetic acid, adamantoic acid, alpha keto glutaric acid, D- or L-aspartic acid, benzensulfonic acid, benzoic acid, 10-camphorsulfunic acid, citric acid, 1,2-ethanedisulfonic acid, fumaric acid, D-gluconic acid, D-glucuronic acid, glucaric acid, D- or L-glutamic acid, glutaric acid, glycolic acid, hippuric acid, hydrobromic acid, hydrochloric acid, 1-hydroxyl-2-napthoic acid, lactobioinic acid, maleic acid, L-malic acid, mandelic acid, methanesulfonic acid, mucic acid, 1,5 napthalenedisulfonic acid tetrahydrate, 2-napthalenesulfonic acid, nitric acid, oleic acid, pamoic acid, phosphoric acid, p-toluenesulfonic acid hydrate, D-saccharid acid monopotassium salt, salicyclic acid, stearic acid, succinic acid, sulfuric acid, tannic acid, D- or L-tartaric acid.

In some embodiments, pharmaceutical salts are formed by the inclusion of counterions (e.g., charged moieties described herein) with particles or conjugates described herein.

Methods of Storing

A therapeutic peptide/protein-polymer conjugate, particle or composition described herein may be stored in a container for at least about 1 hour (e.g., at least about 2 hours, 4 hours, 8 hours, 12 hours, 24 hours, 2 days, 1 week, 1 month, 2 months, 3 months, 4 months, 5 months, 6 months, 1 year, 2 years or 3 years). Accordingly, described herein are containers including a therapeutic peptide/protein-polymer conjugate, particle or composition described herein.

A therapeutic peptide/protein-polymer conjugate, particle or composition may be stored under a variety of conditions, including ambient conditions. A therapeutic peptide/protein-polymer conjugate, particle or composition may also be stored at low temperature, e.g., at a temperature less than or equal to about 5° C. (e.g., less than or equal to about 4° C. or less than or equal to about 0° C.). A polymer-agent conjugate, particle or composition may also be frozen and stored at a temperature of less than about 0° C. (e.g., between −80° C. and −20° C.). A polymer-agent conjugate, particle or composition may also be stored under an inert atmosphere, e.g., an atmosphere containing an inert gas such as nitrogen or argon. Such an atmosphere may be substantially free of atmospheric oxygen and/or other reactive gases, and/or substantially free of moisture.

A therapeutic peptide/protein-polymer conjugate, particle or composition described herein may be stored in a variety of containers, including a light-blocking container such as an amber vial. A container may be a vial, e.g., a sealed vial having a rubber or silicone enclosure (e.g., an enclosure made of polybutadiene or polyisoprene). A container may be substantially free of atmospheric oxygen and/or other reactive gases, and/or substantially free of moisture.

Methods of Evaluating Particles

A particle described herein may be subjected to a number of analytical methods. For example, a particle described herein may be subjected to a measurement to determine whether an impurity or residual solvent is present (e.g., via gas chromatography (GC)), to determine relative amounts of one or more components (e.g., via high performance liquid chromatography (HPLC)), to measure particle size (e.g., via dynamic light scattering and/or scanning electron microscopy), or determine the presence or absence of surface components.

In some embodiments, a particle described herein may be evaluated using dynamic light scattering. Particles may be illuminated with a laser, and the intensity of the scattered light fluctuates at a rate that is dependent upon the size of the particles as smaller particles are “kicked” further by the solvent molecules and move more rapidly. Analysis of these intensity fluctuations yields the velocity of the Brownian motion and hence the particle size using the Stokes-Einstein relationship. The diameter that is measured in Dynamic Light Scattering is called the hydrodynamic diameter and refers to how a particle diffuses within a fluid. The diameter obtained by this technique is that of a sphere that has the same translational diffusion coefficient as the particle being measured.

In some embodiments, a particle described herein may be evaluated using cryo scanning electron microscopy (Cryo-SEM). SEM is a type of electron microscopy in which the sample surface is imaged by scanning it with a high-energy beam of electrons in a raster scan pattern. The electrons interact with the atoms that make up the sample producing signals that contain information about the sample's surface topography, composition and other properties such as electrical conductivity. For Cryo-SEM, the SEM is equipped with a cold stage for cryo-microscopy. Cryofixation may be used and low-temperature scanning electron microscopy performed on the cryogenically fixed specimens. Cryo-fixed specimens may be cryo-fractured under vacuum in a special apparatus to reveal internal structure, sputter coated and transferred onto the SEM cryo-stage while still frozen.

In some embodiments, a particle described herein may be evaluated using transmission electron microscopy (TEM). In this technique, a beam of electrons is transmitted through an ultra thin specimen, interacting with the specimen as it passes through. An image is formed from the interaction of the electrons transmitted through the specimen; the image is magnified and focused onto an imaging device, such as a fluorescent screen, on a layer of photographic film, or to be detected by a sensor such as a charge-coupled device (CCD) camera.

Pharmaceutical Compositions

Provided herein is a composition, e.g., a pharmaceutical composition, comprising a plurality of particles described herein and a pharmaceutically acceptable carrier or adjuvant.

In some embodiments, a pharmaceutical composition may include a pharmaceutically acceptable salt of a compound described herein, e.g., a therapeutic peptide-polymer conjugate. Pharmaceutically acceptable salts of the compounds described herein include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds described herein. Water or oil-soluble or dispersible products may be obtained by such quaternization.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gailate, alpha-tocopherol, and the like; and (3) metal chelating agents, such as citric acid, ethylenediamine tetraacetic acid (EDTA), sorbitol, tartaric acid, phosphoric acid, and the like.

A composition may include a liquid used for suspending a polymer-agent conjugate, particle or composition, which may be any liquid solution compatible with the polymer-agent conjugate, particle or composition, which is also suitable to be used in pharmaceutical compositions, such as a pharmaceutically acceptable nontoxic liquid. Suitable suspending liquids including but are not limited to suspending liquids selected from the group consisting of water, aqueous sucrose syrups, corn syrups, sorbitol, polyethylene glycol, propylene glycol, D5W and mixtures thereof.

A composition described herein may also include another component, such as an antioxidant, antibacterial, buffer, bulking agent, chelating agent, an inert gas, a tonicity agent and/or a viscosity agent.

In one embodiment, the polymer-agent conjugate, particle or composition is provided in lyophilized form and is reconstituted prior to administration to a subject. The lyophilized polymer-agent conjugate, particle or composition can be reconstituted by a diluent solution, such as a salt or saline solution, e.g., a sodium chloride solution having a pH between 6 and 9, lactated Ringer's injection solution, or a commercially available diluent, such as PLASMA-LYTE A Injection pH 7.4® (Baxter, Deerfield, Ill.).

In one embodiment, a lyophilized formulation includes a lyoprotectant or stabilizer to maintain physical and chemical stability by protecting the particle and active from damage from crystal formation and the fusion process during freeze-drying. The lyoprotectant or stabilizer can be one or more of polyethylene glycol (PEG), a PEG lipid conjugate (e.g., PEG-ceramide or D-alpha-tocopheryl polyethylene glycol 1000 succinate), poly(vinyl alcohol) (PVA), poly(vinylpyrrolidone) (PVP), polyoxyethylene esters, poloxamers, polysorbates, polyoxyethylene esters, lecithins, saccharides, oligosaccharides, polysaccharides, carbohydrates, cyclodextrins (e.g. 2-hydroxypropyl-β-cyclodextrin) and polyols (e.g., trehalose, mannitol, sorbitol, lactose, sucrose, glucose and dextran), salts and crown ethers.

In some embodiments, the lyophilized polymer-agent conjugate, particle or composition is reconstituted with water, 5% Dextrose Injection, Lactated Ringer's and Dextrose Injection, or a mixture of equal parts by volume of Dehydrated Alcohol, USP and a nonionic surfactant, such as a polyoxyethylated castor oil surfactant available from GAF Corporation, Mount Olive, N.J., under the trademark, Cremophor EL. The lyophilized product and vehicle for reconstitution can be packaged separately in appropriately light-protected vials. To minimize the amount of surfactant in the reconstituted solution, only a sufficient amount of the vehicle may be provided to form a solution of the polymer-agent conjugate, particle or composition. Once dissolution of the drug is achieved, the resulting solution is further diluted prior to injection with a suitable parenteral diluent. Such diluents are well known to those of ordinary skill in the art. These diluents are generally available in clinical facilities. It is, however, within the scope of the present invention to package the subject polymer-agent conjugate, particle or composition with a third vial containing sufficient parenteral diluent to prepare the final concentration for administration. A typical diluent is Lactated Ringer's Injection.

The final dilution of the reconstituted polymer-agent conjugate, particle or composition may be carried out with other preparations having similar utility, for example, 5% Dextrose Injection, Lactated Ringer's and Dextrose Injection, Sterile Water for Injection, and the like. However, because of its narrow pH range, pH 6.0 to 7.5, Lactated Ringer's Injection is most typical. Per 100 mL, Lactated Ringer's Injection contains Sodium Chloride USP 0.6 g, Sodium Lactate 0.31 g, Potassium chloride USP 0.03 g and Calcium Chloride2H2O USP 0.02 g. The osmolarity is 275 mOsmol/L, which is very close to isotonicity.

The compositions may conveniently be presented in unit dosage form and may be prepared by any methods well known in the art of pharmacy. The amount of active agent which can be combined with a pharmaceutically acceptable carrier to produce a single dosage form will vary depending upon the host being treated, the particular mode of administration. The amount of active agent which can be combined with a pharmaceutically acceptable carrier to produce a single dosage form will generally be that amount of the compound which produces a therapeutic effect.

Routes of Administration

The pharmaceutical compositions described herein may be administered orally, parenterally (e.g., via intravenous, subcutaneous, intracutaneous, intramuscular, intraarticular, intraarterial, intrasynovial, intrasternal, intrathecal, intralesional, intraocular, or intracranial injection), topically, mucosally (e.g., rectally or vaginally), nasally, buccally, ophthalmically, via inhalation spray (e.g., delivered via nebulzation, propellant or a dry powder device) or via an implanted reservoir.

Pharmaceutical compositions suitable for parenteral administration comprise one or more polymer-agent conjugate(s), particle(s) or composition(s) in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the agent from subcutaneous or intramuscular injection. This may be accomplished by the use of a liquid suspension of crystalline or amorphous material having poor water solubility. The rate of absorption of the polymer-agent conjugate, particle or composition then depends upon its rate of dissolution which, in turn, may depend upon crystal size and crystalline form. Alternatively, delayed absorption of a parenterally administered drug form is accomplished by dissolving or suspending the polymer-agent conjugate, particle or composition in an oil vehicle.

Pharmaceutical compositions suitable for oral administration may be in the form of capsules, cachets, pills, tablets, gums, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of an agent as an active ingredient. A compound may also be administered as a bolus, electuary or paste.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the polymer-agent conjugate, particle or composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the polymer-agent conjugate, particle or composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for topical administration are useful when the desired treatment involves areas or organs readily accessible by topical application. For application topically to the skin, the pharmaceutical composition should be formulated with a suitable ointment containing the active components suspended or dissolved in a carrier. Carriers for topical administration of the a particle described herein include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active particle suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions described herein may also be topically applied to the lower intestinal tract by rectal suppository formulation or in a suitable enema formulation. Topically-transdermal patches are also included herein.

The pharmaceutical compositions described herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The pharmaceutical compositions described herein may also be administered in the form of suppositories for rectal or vaginal administration. Suppositories may be prepared by mixing one or more polymer-agent conjugate, particle or composition described herein with one or more suitable non-irritating excipients which is solid at room temperature, but liquid at body temperature. The composition will therefore melt in the rectum or vaginal cavity and release the polymer-agent conjugate, particle or composition. Such materials include, for example, cocoa butter, polyethylene glycol, a suppository wax or a salicylate. Compositions of the present invention which are suitable for vaginal administration also include pessaries, tampons, creams, gels, pastes, foams or spray formulations containing such carriers as are known in the art to be appropriate.

Ophthalmic formulations, eye ointments, powders, solutions and the like, are also contemplated as being within the scope of the invention. An ocular tissue (e.g., a deep cortical region, a supranuclear region, or an aqueous humor region of an eye) may be contacted with the ophthalmic formulation, which is allowed to distribute into the lens. Any suitable method(s) of administration or application of the ophthalmic formulations of the invention (e.g., topical, injection, parenteral, airborne, etc.) may be employed. For example, the contacting may occur via topical administration or via injection.

Dosages and Dosage Regimens

The therapeutic peptide/protein-polymer conjugates, particles or compositions can be formulated into pharmaceutically acceptable dosage forms by conventional methods known to those of skill in the art.

Actual dosage levels of the active ingredients in the pharmaceutical compositions of this invention may be varied so as to obtain an amount of the therapeutic peptide which is effective to achieve the desired therapeutic response for a particular subject, composition, and mode of administration, without being toxic to the subject.

In one embodiment, the therapeutic peptide/protein-polymer conjugate, particle or composition is administered to a subject at a dosage of, e.g., about 0.1 to 300 mg/m², about 5 to 275 mg/m², about 10 to 250 mg/m², e.g., about 15, 20, 25, 30, 35, 40, 45, 50, 55, 60, 65, 70, 80, 90, 100, 110, 120, 130, 140, 150, 160, 170, 180, 190, 200, 210, 220, 230, 240, 250, 260, 270, 280, 290 mg/m². Administration can be at regular intervals, such as every 1, 2, 3, 4, or 5 days, or weekly, or every 2, 3, 4, 5, 6, or 7 or 8 weeks. The administration can be over a period of from about 10 minutes to about 6 hours, e.g., from about 30 minutes to about 2 hours, from about 45 minutes to 90 minutes, e.g., about 30 minutes, 45 minutes, 1 hour, 2 hours, 3 hours, 4 hours, 5 hours or more. In one embodiment, the therapeutic peptide-polymer conjugate, particle or composition is administered as a bolus infusion or intravenous push, e.g., over a period of 15 minutes, 10 minutes, 5 minutes or less. In one embodiment, therapeutic peptide-polymer conjugate, particle or composition is administered in an amount such the desired dose of the agent is administered. Preferably the dose of the therapeutic peptide/protein-polymer conjugate, particle or composition is a dose described herein.

In one embodiment, the subject receives 1, 2, 3, up to 10, up to 12, up to 15 treatments, or more, or until the disorder or a symptom of the disorder is cured, healed, alleviated, relieved, altered, remedied, ameliorated, palliated, improved or affected. For example, the subject receive an infusion once every 1, 2, 3 or 4 weeks until the disorder or a symptom of the disorder are cured, healed, alleviated, relieved, altered, remedied, ameliorated, palliated, improved or affected. Preferably, the dosing schedule is a dosing schedule described herein.

The therapeutic peptide/protein-polymer, particle, or composition can be administered as a first line therapy, e.g., alone or in combination with an additional agent or agents. In other embodiments, a therapeutic peptide/protein-polymer conjugate, particle or composition is administered after a subject has developed resistance to, has failed to respond to or has relapsed after a first line therapy. The therapeutic peptide/protein-polymer conjugate, particle or composition may be administered in combination with a second agent. Preferably, the therapeutic peptide/protein-polymer conjugate, particle or composition is administered in combination with a second agent described herein. The second agent may be the same or different as the agent in the particle.

Kits

A therapeutic peptide/protein-polymer conjugate, particle or composition described herein may be provided in a kit. The kit includes a therapeutic peptide/protein-polymer conjugate, particle or composition described herein and, optionally, a container, a pharmaceutically acceptable carrier and/or informational material. The informational material can be descriptive, instructional, marketing or other material that relates to the methods described herein and/or the use of the particles for the methods described herein.

The informational material of the kits is not limited in its form. In one embodiment, the informational material can include information about production of the therapeutic peptide/protein-polymer conjugate, particle or composition, physical properties of the therapeutic peptide/protein-polymer conjugate, particle or composition, concentration, date of expiration, batch or production site information, and so forth. In one embodiment, the informational material relates to methods for administering the therapeutic peptide/protein-polymer conjugate, particle or composition.

In one embodiment, the informational material can include instructions to administer a therapeutic peptide/protein-polymer conjugate, particle or composition described herein in a suitable manner to perform the methods described herein, e.g., in a suitable dose, dosage form, or mode of administration (e.g., a dose, dosage form, or mode of administration described herein). In another embodiment, the informational material can include instructions to administer a therapeutic peptide/protein-polymer conjugate, particle or composition described herein to a suitable subject, e.g., a human, e.g., a human having or at risk for a disorder described herein. In another embodiment, the informational material can include instructions to reconstitute a therapeutic peptide/protein-polymer conjugate or particle described herein into a pharmaceutically acceptable composition.

In one embodiment, the kit includes instructions to use the therapeutic peptide/protein-polymer conjugate, particle or composition, such as for treatment of a subject. The instructions can include methods for reconstituting or diluting the therapeutic peptide-polymer conjugate, particle or composition for use with a particular subject or in combination with a particular chemotherapeutic agent. The instructions can also include methods for reconstituting or diluting the therapeutic peptide/protein-polymer composition for use with a particular means of administration, such as by intravenous infusion.

In another embodiment, the kit includes instructions for treating a subject with a particular indication, such as a particular cancer.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a particle described herein and/or its use in the methods described herein. The informational material can also be provided in any combination of formats.

In addition to a therapeutic peptide/protein-polymer conjugate, particle or composition described herein, the composition of the kit can include other ingredients, such as a surfactant, a lyoprotectant or stabilizer, an antioxidant, an antibacterial agent, a bulking agent, a chelating agent, an inert gas, a tonicity agent and/or a viscosity agent, a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, a pharmaceutically acceptable carrier and/or a second agent for treating a condition or disorder described herein. Alternatively, the other ingredients can be included in the kit, but in different compositions or containers than a particle described herein. In such embodiments, the kit can include instructions for admixing a polymer-agent conjugate, particle or composition described herein and the other ingredients, or for using a therapeutic peptide/protein-polymer conjugate, particle or composition described herein together with the other ingredients.

In another embodiment, the kit includes a second therapeutic agent, such as a second chemotherapeutic. In one embodiment, the second agent is in lyophilized or in liquid form. In one embodiment, the therapeutic peptide/protein-polymer conjugate, particle or composition and the second therapeutic agent are in separate containers, and in another embodiment, the therapeutic peptide/protein-polymer conjugate, particle or composition and the second therapeutic agent are packaged in the same container.

In some embodiments, a component of the kit is stored in a sealed vial, e.g., with a rubber or silicone enclosure (e.g., a polybutadiene or polyisoprene enclosure). In some embodiments, a component of the kit is stored under inert conditions (e.g., under Nitrogen or another inert gas such as Argon). In some embodiments, a component of the kit is stored under anhydrous conditions (e.g., with a desiccant). In some embodiments, a component of the kit is stored in a light blocking container such as an amber vial.

A therapeutic peptide/protein-polymer conjugate, particle or composition described herein can be provided in any form, e.g., liquid, frozen, dried or lyophilized form. It is preferred that a polymer-agent conjugate, particle or composition described herein be substantially pure and/or sterile. In an embodiment, the therapeutic peptide/protein-polymer conjugate, particle or composition is sterile. When a therapeutic peptide/protein-polymer conjugate, particle or composition described herein is provided in a liquid solution, the liquid solution preferably is an aqueous solution, with a sterile aqueous solution being preferred. In one embodiment, the therapeutic peptide/protein-polymer conjugate, particle or composition is provided in lyophilized form and, optionally, a diluent solution is provided for reconstituting the lyophilized agent. The diluent can include for example, a salt or saline solution, e.g., a sodium chloride solution having a pH between 6 and 9, lactated Ringer's injection solution, D5W, or PLASMA-LYTE A Injection pH 7.4® (Baxter, Deerfield, Ill.).

The kit can include one or more containers for the composition containing a therapeutic peptide/protein-polymer conjugate, particle or composition described herein. In some embodiments, the kit contains separate containers, dividers or compartments for the composition and informational material. For example, the composition can be contained in a bottle, vial, IV admixture bag, IV infusion set, piggyback set or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a polymer-agent conjugate, particle or composition described herein. For example, the kit includes a plurality of syringes, ampules, foil packets, or blister packs, each containing a single unit dose of a particle described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device.

In one embodiment, the device is a medical implant device, e.g., packaged for surgical insertion.

Methods of Using Particles and Compositions

The polymer-agent conjugates, particles and compositions described herein can be administered to cells in culture, e.g. in vitro or ex vivo, or to a subject, e.g., in vivo, to treat or prevent a variety of disorders, including those described herein below. The polymer-agent conjugates, particles and compositions can be used as part of a first line, second line, or adjunct therapy, and can also be used alone or in combination with one or more additional treatment regimes.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Purification and Characterization of 5050 PLGA

Step A: A 3-L round-bottom flask equipped with a mechanical stirrer was charged with 5050PLGA (300 g, Mw: 7.8 kDa; Mn: 2.7 kDa) and acetone (900 mL). The mixture was stirred for 1 h at ambient temperature to form a clear yellowish solution.

Step B: A 22-L jacket reactor with a bottom-outlet valve equipped with a mechanical stirrer was charged with MTBE (9.0 L, 30 vol. to the mass of 5050 PLGA). Celite® (795 g) was added to the solution with overhead stirring at ˜200 rpm to produce a suspension. To this suspension was slowly added the solution from Step A over 1 h. The mixture was agitated for an additional one hour after addition of the polymer solution and filtered through a polypropylene filter. The filter cake was washed with MTBE (3×300 mL), conditioned for 0.5 h, air-dried at ambient temperature (typically 12 h) until residual MTBE was ≦5 wt % (as determined by ¹H NMR analysis.

Step C: A 12-L jacket reactor with a bottom-outlet valve equipped with a mechanical stirrer was charged with acetone (2.1 L, 7 vol. to the mass of 5050 PLGA). The polymer/Celite® complex from Step B was charged into the reactor with overhead stirring at ˜200 rpm to produce a suspension. The suspension was stirred at ambient temperature for an additional 1 h and filtered through a polypropylene filter. The filter cake was washed with acetone (3×300 mL) and the combined filtrates were clarified through a 0.45 mM in-line filter to produce a clear solution. This solution was concentrated to ˜1000 mL.

Step D: A 22-L jacket reactor with a bottom-outlet valve equipped with a mechanical stirrer was charged with water (9.0 L, 30 vol.) and was cooled down to 0-5° C. using a chiller. The solution from Step C was slowly added over 2 h with overhead stirring at ˜200 rpm. The mixture was stirred for an additional one hour after addition of the solution and filtered through a polypropylene filter. The filter cake was conditioned for 1 h, air-dried for 1 day at ambient temperature, and then vacuum-dried for 3 days to produce the purified 5050 PLGA as a white powder [258 g, 86% yield]. The ¹H NMR analysis was consistent with that of the desired product and Karl Fisher analysis showed 0.52 wt % of water. The product was analyzed by HPLC (AUC, 230 nm) and GPC (AUC, 230 nm). The process produced a narrower polymer polydispersity, i.e. Mw: 8.8 kDa and Mn: 5.8 kDa.

### Example 2

### Purification and Characterization of 5050 PLGA Lauryl Ester

A 12-L round-bottom flask equipped with a mechanical stirrer was charged with MTBE (4 L) and heptanes (0.8 L). The mixture was agitated at ˜300 rpm, to which a solution of 5050 PLGA lauryl ester (65 g) in acetone (300 mL) was added dropwise. Gummy solids were formed over time and finally clumped up on the bottom of the flask. The supernatant was decanted off and the solid was dried under vacuum at 25° C. for 24 h to afford 40 g of purified 5050 PLGA lauryl ester as a white powder [yield: 61.5%]. ¹H NMR (CDCl₃, 300 MHz): δ 5.25-5.16 (m, 53H), 4.86-4.68 (m, 93H), 4.18 (m, 7H), 1.69-1.50 (m, 179H), 1.26 (bs, 37H), 0.88 (t, J=6.9 Hz, 6H). The ¹H NMR analysis was consistent with that of the desired product. GPC (AUC, 230 nm): 6.02-9.9 min, t_{R}=7.91 min

### Example 3

### Purification and Characterization of 7525 PLGA

A 22-L round-bottom flask equipped with a mechanical stirrer was charged with 12 L of MTBE, to which a solution of 7525 PLGA (150 g, approximately 6.6 kDa) in dichloromethane (DCM, 750 mL) was added dropwise over an hour with an agitation of ˜300 rpm, resulting in a gummy solid. The supernatant was decanted off and the gummy solid was dissolved in DCM (3 L). The solution was transferred to a round-bottom flask and concentrated to a residue, which was dried under vacuum at 25° C. for 40 h to afford 94 g of purified 7525 PLGA as a white foam [yield: 62.7%, 1. ¹H NMR (CDCl₃, 300 MHz): δ 5.24-5.15 (m, 68H), 4.91-4.68 (m, 56H), 3.22 (s, 2.3H, MTBE), 1.60-1.55 (m, 206H), 1.19 (s, 6.6H, MTBE). The ¹H NMR analysis was consistent with that of the desired product. GPC (AUC, 230 nm): 6.02-9.9 min, t_{R}=7.37 min.

### Example 4

### Synthesis, Purification and Characterization of O-acetyl-5050-PLGA

A 2000-mL, round-bottom flask equipped with an overhead stirrer was charged with purified 5050 PLGA [220 g, Mn of 57001 and DCM (660 mL). The mixture was stirred for 10 min to form a clear solution. Ac₂O (11.0 mL, 116 mmol) and pyridine (9.4 mL, 116 mmol) were added to the solution, resulting in a minor exotherm of ˜0.5° C. The reaction was stirred at ambient temperature for 3 h and concentrated to ˜600 mL. The solution was added to a suspension of Celite® (660 g) in MTBE (6.6 L, 30 vol.) over 1 h with overhead stirring at ˜200 rpm. The suspension was filtered through a polypropylene filter and the filter cake was air-dried at ambient temperature for 1 day. It was suspended in acetone (1.6 L, ˜8 vol) with overhead stirring for 1 h. The slurry was filtered though a fritted funnel (coarse) and the filter cake was washed with acetone (3×300 mL). The combined filtrates were clarified though a Celite® pad to afford a clear solution. It was concentrated to ˜700 mL and added to cold water (7.0 L, 0-5° C.) with overhead stirring at 200 rpm over 2 h. The suspension was filtered though a polypropylene filter. The filter cake was washed with water (3×500 mL), and conditioned for 1 h to afford 543 g of wet cake. It was transferred to two glass trays and air-dried at ambient temperature overnight to afford 338 g of wet product, which was then vacuum-dried at 25° C. for 2 days to constant weight to afford 201 g of product as a white powder [yield: 91%]. The ¹H NMR analysis was consistent with that of the desired product. The product was analyzed by HPLC (AUC, 230 nm) and GPC (Mw: 9.0 kDa and Mn: 6.3 kDa).

### Example 5

### Synthesis, Purification and Characterization of Folate-PEG-PLGA-Lauryl Ester

The synthesis of folate-PEG-PLGA-lauryl ester involves the direct coupling of folic acid to PEG bisamine (Sigma-Aldrich, n=75, MW 3350 Da). PEG bisamine was purified due to the possibility that small molecular weight amines were present in the product. 4.9 g of PEG bisamine was dissolved in DCM (25 mL, 5 vol) and then transferred into MTBE (250 mL, 50 vol) with vigorous agitation. The polymer precipitated as white powder. The mixture was then filtered and the solid was dried under vacuum to afford 4.5 g of the product [92%]. The ¹H NMR analysis of the solid gave a clean spectrum; however, not all alcohol groups were converted to amines based on the integration of α-methylene to the amine group (63% bisamine, 37% monoamine).

Folate-(γ)CO—NH-PEG-NH₂ was synthesized using the purified PEG bisamine. Folic acid (100 mg, 1.0 equiv.) was dissolved in hot DMSO (4.5 mL, 3 vol to PEG bisamine). The solution was cooled to ambient temperature and (2-(7-Aza-1H-benzotriazole-1-yl)-1,1,3,3-tetramethyluronium hexafluorophosphate) (HATU, 104 mg, 1.2 equiv.) and N,N-Diisopropylethylamine (DIEA, 80 μL, 2.0 equiv.) were added. The resulting yellow solution was stirred for 30 minutes and PEG bisamine (1.5 g, 2 equiv.) in DMSO (3 mL, 2 vol) was added. Excess PEG bisamine was used to avoid the possible formation of di-adduct of PEG bisamine and to improve the conversion of folic acid. The reaction was stirred at 20° C. for 16 h and directly purified by CombiFlash® using a C18 column (RediSep, 43 g, C18). The fractions containing the product were combined and the CH₃CN was removed under vacuum. The remaining water solution (−200 mL) was extracted with chloroform (200 mL×2). The combined chloroform phases were concentrated to approximately 10 mL and transferred into MTBE to precipitate the product as a yellow powder. In order to completely remove any unreacted PEG bisamine in the material, the yellow powder was washed with acetone (200 mL) three times. The remaining solid_was dried under vacuum to afford a yellow semi-solid product (120 mg). HPLC analysis indicated a purity of 97% and the ¹H NMR analysis showed that the product was clean.

Folate-(γ)CO—NH-PEG-NH₂ was reacted with p-nitrophenyl-COO-PLGA-CO₂-lauryl to provide folic acid-PEG-PLGA-lauryl ester. To prepare p-nitrophenyl-COO-PLGA-CO₂-lauryl, PLGA 5050 (lauryl ester) [10.0 g, 1.0 equiv.] and p-nitrophenyl chloroformate (0.79 g, 2.0 equiv.) were dissolved in DCM. To the dissolved polymer solution, one portion of TEA (3.0 equiv.) was added. The resulting solution was stirred at 20° C. for 2 h and the ¹H NMR analysis indicated complete conversion. The reaction solution was then transferred into a solvent mixture of 4:1 MTBE/heptanes (50 vol). The product precipitated and gummed up. The supernatant was decanted off and the solid was dissolved in acetone (20 vol). The resulting acetone suspension was filtered and the filtrate was concentrated to dryness to produce the product as a white foam [7.75 g, 78%, Mn=4648 based on GPC]. The ¹H NMR analysis indicated a clean product with no detectable p-nitrophenol.

Folate-(γ)CO—NH-PEG-NH₂ (120 mg, 1.0 equiv.) was dissolved in DMSO (5 mL) and TEA (3.0 equiv.) was added. The pH of the reaction mixture was 8-9. p-nitrophenyl-COO-PLGA-CO₂-lauryl (158 mg, 1.0 equiv.) in DMSO (1 mL) was added and the reaction was monitored by HPLC. A new peak at 16.1 min (−40%, AUC, 280 nm) was observed from the HPLC chromatogram in 1 h. A small sample of the reaction mixture was treated with excess 1,8-diazabicyclo[5.4.0]undec-7-ene (DBU) and the color instantly changed to dark yellow. HPLC analysis of this sample indicated complete disappearance of p-nitrophenyl-COO-PLGA-CO₂-lauryl and the 16.1 min peak. Instead, a peak on the right side of folate-(γ)CO—NH-PEG-NH₂ appeared. It can be concluded that the p-nitrophenyl-COO-PLGA-CO₂-lauryl and the possible product were not stable under strong basic conditions. In order to identify the new peak at 16.1 min, ˜1/3 of the reaction mixture was purified by CombiFlash®. The material was finally eluted with a solvent mixture of 1:4 DMSO/CH₃CN. It was observed that this material was yellow which could have indicated folate content. Due to the large amount of DMSO present, this material was not isolated from the solution. The fractions containing unreacted folate-(γ)CO—NH-PEG-NH₂ was combined and concentrated to a residue. A ninhydrin test of this residue gave a negative result, which may imply the lack of amine group at the end of the PEG. This observation can also explain the incomplete conversion of the reaction.

The rest of reaction solution was purified by CombiFlash®. Similarly to the previous purification, the suspected yellow product was retained by the column MeOH containing 0.5% TFA was used to elute the material. The fractions containing the possible product were combined and concentrated to dryness. The ¹H NMR analysis of this sample indicated the existence of folate, PEG and lauryl-PLGA and the integration of these segments was close to the desired value of 1:1:1 ratio of all three components. High purities were observed from both HPLC and GPC analyses. The Mn based on GPC was 8.7 kDa. The sample in DMSO was recovered by precipitation into MTBE.

### Example 6

### Synthesis of PLGA-PEG-PLGA Therapeutic Peptide Conjugate

The triblock copolymer PLGA-PEG-PLGA will be synthesized using a method developed by Zentner et al., Journal of Controlled Release, 72, 2001, 203-215. The molecular weight of PLGA obtained using this method will be ˜3 kDa. A similar method reported by Chen et al., International Journal of Pharmaceutics, 288, 2005, 207-218 will be used to synthesize PLGA molecular weights ranging from 1-7 kDa. The LA/GA ratio will typically be, but is not limited to, a ratio of 1:1. The minimum PEG molecular weight will be 2 kDa with an upper limit of 30 kDa. The preferred range of PEG will be 3-12 kDa. The PLGA molecular weight will be a minimum value of 4 kDa and a maximum of 30 kDa. The preferred range of PLGA will be 7-20 kDa. A therapeutic peptide (e.g., histrelin or thymopentin) could be conjugated to the PLGA through an appropriate linker (i.e., as listed in the examples) to form a polymer-therapeutic peptide conjugate. In addition, the same therapeutic peptide or a different therapeutic peptide could be attached to the other PLGA to form a dual therapeutic peptide polymer conjugate with two same therapeutic peptides or two different therapeutic peptides. Nanoparticles could be formed from either the PLGA-PEG-PLGA alone or from a single therapeutic peptide or dual therapeutic peptide polymer conjugate composed of this triblock copolymer.

### Example 7

### Synthesis of polycaprolactone-poly(ethylene glycol)-polycaprolactone (PCL-PEG-PCL) Therapeutic Peptide Conjugate

The triblock PCL-PEG-PCL will be synthesized using a ring open polymerization method in the presence of a catalyst (i.e., stannous octoate) as reported in Hu et al., Journal of Controlled Release, 118, 2007, 7-17. The molecular weights of PCL obtained from this synthesis will range from 2 to 22 kDa. A non-catalyst method shown in the article by Ge et al. Journal of Pharmaceutical Sciences, 91, 2002, 1463-1473 will also be used to synthesize PCL-PEG-PCL. The molecular weights of PCL that will be obtained from this particular synthesis range from 9 to 48 kDa. Similarly, another catalyst free method developed by Cerrai et al., Polymer, 30, 1989, 338-343 will be used to synthesize the triblock copolymer with molecular weights of PCL ranging from 1-9 kDa. The minimum PEG molecular weight will be 2 kDa with an upper limit of 30 kDa. The preferred range of PEG will be 3-12 kDa. The PCL molecular weight will be a minimum value of 4 kDa and a maximum of 30 kDa. The preferred range of PCL would be 7-20 kDa. A therapeutic peptide (e.g., histrelin or thymopentin) could be conjugated to the PCL through an appropriate linker (i.e., as listed in the examples) to form a polymer-therapeutic peptide conjugate. In addition, the same therapeutic peptide or a different therapeutic peptide could be attached to the other PCL to form a dual therapeutic peptide polymer conjugate with two same therapeutic peptides or two different therapeutic peptides. Nanoparticles could be formed from either the PCL-PEG-PCL alone or from a single therapeutic peptide or dual therapeutic peptide polymer conjugate composed of this triblock copolymer.

### Example 8

### Synthesis of polylactide-poly(ethylene glycol)-polylactide (PLA-PEG-PLA) Therapeutic Peptide Conjugate

The triblock PLA-PEG-PLA copolymer will be synthesized using a ring opening polymerization using a catalyst (i.e. stannous octoate) reported in Chen et al., Polymers for Advanced Technologies, 14, 2003, 245-253. The molecular weights of PLA that will be formed range from 6 to 46 kDa. A lower molecular weight range (i.e. 1-8 kDa) could be achieved by using the method shown by Zhu et al., Journal of Applied Polymer Science, 39, 1990, 1-9. The minimum PEG molecular weight will be 2 kDa with an upper limit of 30 kDa. The preferred range of PEG will be 3-12 kDa. The PLA molecular weight will be a minimum value of 4 kDa and a maximum of 30 kDa. The preferred range of PLA will be 7-20 kDa. A therapeutic peptide (e.g., histrelin or thymopentin) could be conjugated to the PLA through an appropriate linker (i.e., as listed in the examples) to form a polymer-therapeutic peptide conjugate. In addition, the same therapeutic peptide or a different therapeutic peptide could be attached to the other PLA to form a dual therapeutic peptide polymer conjugate with two same therapeutic peptides or two different therapeutic peptides. Nanoparticles could be formed from either the PLA-PEG-PLA alone or from a single therapeutic peptide or dual therapeutic peptide polymer conjugate composed of this triblock copolymer.

### Example 9

### Synthesis of p-dioxanone-co-lactide-poly(ethylene glycol)-p-dioxanone-co-lactide (PDO-PEG-PDO) Therapeutic Peptide Conjugate

The triblock PDO-PEG-PDO will be synthesized in the presence of a catalyst (stannous 2-ethylhexanoate) using a method developed by Bhattari et al., Polymer International, 52, 2003, 6-14. The molecular weight of PDO obtained from this method ranges from 2-19 kDa. The minimum PEG molecular weight will be 2 kDa with an upper limit of 30 kDa. The preferred range of PEG would be 3-12 kDa. The PDO molecular weight will be a minimum value of 4 kDa and a maximum of 30 kDa. The preferred range of PDO will be 7-20 kDa. A therapeutic peptide (e.g., histrelin or thymopentin) could be conjugated to the PDO through an appropriate linker (i.e., as listed in the examples) to form a polymer-therapeutic peptide conjugate. In addition, the same therapeutic peptide or a different therapeutic peptide could be attached to the other PDO to form a dual therapeutic peptide polymer conjugate with two same therapeutic peptides or two different therapeutic peptides. Nanoparticles could be formed from either the PDO-PEG-PDO alone or from a single therapeutic peptide or dual therapeutic peptide polymer conjugate composed of this triblock copolymer.

### Example 10

### Synthesis of polyfunctionalized PLGA/PLA Based Polymers

One could synthesize a PLGA/PLA related polymer with functional groups that are dispersed throughout the polymer chain that is readily biodegradable and whose components are all bioacceptable components (i.e. known to be safe in humans). Specifically, PLGA/PLA related polymers derived from 3-S-[benxyloxycarbonyl)methyl]-1,4-dioxane-2,5-dione (BMD) could be synthesized (see structures below). (The structures below are intended to represent random copolymers of the monomeric units shown in brackets.) Exemplary R groups include a negative charge, H, alkyl, and arylalkyl.

1. PLGA/PLA related polymer derived from BMD

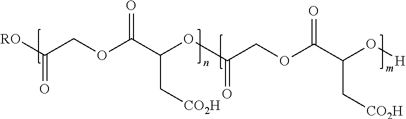

2. PLGA/PLA related polymer with BMD and 3,5-dimethyl-1,4-dioxane-2,5-dione (bis-DL-lactic acid cyclic diester)

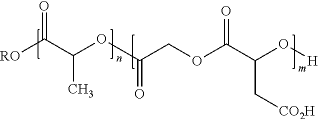

3. PLGA/PLA related polymer with BMD and 1,4-dioxane-2,5-dione (bis-glycolic acid cyclic diester

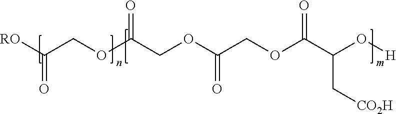

In a preferred embodiment, PLGA/PLA polymers derived from BMD and bis-DL-lactic acid cyclic diester will be prepared with a number of different pendent functional groups by varying the ratio of BMD and lactide. For reference, if it is assumed that each polymer has a number average molecular weight (Mn) of 8 kDa, then a polymer that is 100 wt % derived from BMD has approximately 46 pendant carboxylic acid groups (1 acid group per 0.174 kDa). Similarly, a polymer that is 25 wt % derived from BMD and 75 wt % derived from 3,5-dimethyl-1,4-dioxane-2,5-dione (bis-DL-lactic acid cyclic diester) has approximately 11 pendant carboxylic acid groups (1 acid group per 0.35 kDa). This compares to just 1 acid group for an 8 kDa PLGA polymer that is not functionalized and 1 acid group/2 kDa if there are 4 sites added during functionalization of the terminal groups of a linear PLGA/PLA polymer or 1 acid group/1 kDa if a 4 kDa molecule has four functional groups attached.

Specifically, the PLGA/PLA related polymers derived from BMD will be developed using a method by Kimura et al., Macromolecules, 21, 1988, 3338-3340. This polymer will have repeating units of glycolic and malic acid with a pendant carboxylic acid group on each unit [RO(COCH₂OCOCHR₁O)ₙH where R is H, or alkyl or PEG unit, etc., and R₁ is CO₂H]. There is one pendant carboxylic acid group for each 174 mass units. The molecular weight of the polymer and the polymer polydispersity can vary with different reaction conditions (i.e. type of initiator, temperature, processing condition). The Mn could range from 2 to 21 kDa. Also, there will be a pendant carboxylic acid group for every two monomer components in the polymer. Based on the reference previously sited, NMR analysis showed no detectable amount of the β-malate polymer was produced by ester exchange or other mechanisms.

Another type of PLGA/PLA related polymer derived from BMD and 3,5-dimethyl-1,4-dioxane-2,5-dione (bis-DL-lactic acid cyclic diester) will be synthesized using a method developed by Kimura et al., Polymer, 1993, 34, 1741-1748. They showed that the highest BMD ratio utilized was 15 mol % and this translated into a polymer containing 14 mol % (16.7 wt %) of BMD-derived units. This level of BMD incorporation represents approximately 8 carboxylic acid residues per 8 kDa polymer (1 carboxylic acid residue/kDa of polymer). Similarly to the use of BMD alone, no β-malate derived polymer was detected. Also, Kimura et al. reported that the glass transition temperatures (T_{g}) were in the low 20° C. despite the use of high polymer molecular weights (36-67 kDa). The T_{g}'s were in the 20-23° C. for these polymers whether the carboxylic acid was free or still a benzyl group. The inclusion of more rigidifying elements (i.e. carboxylic acids which can form strong hydrogen bonds) should increase the T_{g}. Possible prevention of aggregation of any particles formed from a polymer drug conjugate derived from this specific polymer will have to be evaluated due to possible lower T_{g} values.

Another method for synthesizing a PLA-PEG polymer that contains varying amounts of glycolic acid malic acid benzyl ester involves the polymerization of BMD in the presence of 3,5-dimethyl-1,4-dioxane-2,5-dione (bis-DL-lactic acid cyclic diester), reported by Lee et al., Journal of Controlled Release, 94, 2004, 323-335. They reported that the synthesized polymers contained 1.3-3.7 carboxylic acid units in a PLA chain of approximately 5-8 kDa (total polymer weight was approximately 11-13 kDa with PEG being 5 kDa) depending on the quantity of BMD used in the polymerization. In one polymer there were 3.7 carboxylic acid units/hydrophobic block in which the BMD represents approximately 19 wt % of the weight of the hydrophobic block. The ratio of BMD to lactide was similar to that observed by Kimura et al., Polymer, 1993, 34, 1741-1748 and the acid residues were similar in the resulting polymers (approximately 1 acid unit/kDa of hydrophobic polymer).

Polymers functionalized with BMD that are more readily hydrolysable will be prepared using the method developed by Kimura et al., International Journal of Biological Macromolecules, 25, 1999, 265-271. They reported that the rate of hydrolysis was related to the number of free acid groups present (with polymers with more acid groups hydrolyzing faster). The polymers had approximately 5 or 10 mol % BMD content. Also, in the reference by Lee et al., Journal of Controlled Release, 94, 2004, 323-335, the rate of hydrolysis of the polymer was fastest with the highest concentration of pendent acid groups (6 days for polymer containing 19.5 wt % of BMD and 20 days for polymer containing 0 wt % of BMD.

A therapeutic peptide (e.g., histrelin or thymopentin) could be conjugated to a PLGA/PLA related polymer with BMD (refer to previous examples above). Similarly, a particle could be prepared from such a polymer therapeutic peptide conjugate.

### Example 11

### Synthesis of Polymers Prepared Using β-Lactone of Malic Acid Benzyl Esters

One could prepare a polymer by polymerizing MePEGOH with RS-β-benzyl malolactonate (a β-lactone) with DL-lactide (cyclic diester of lactic acid) to afford a polymer containing MePEG (lactic acid) (malic acid) Me(OCH₂CH₂O)[OCCCH(CH₃)O]ₘ[COCH₂CH(CO₂H)O] as developed by Wang et al., Colloid Polymer Sci., 2006, 285, 273-281. These polymers will potentially degrade faster because they contain higher levels of acidic groups. It should be noted that the use of β-lactones generate a different polymer from that obtained using 3-[(benzyloxycarbonyl)methyl]-1,4-dioxane-2,5-dione. In these polymers, the carboxylic acid group is directly attached to the polymer chain without a methylene spacer.

Another polymer that could be prepared directly from a β-lactone was reported by Ouhib et al., Ch. Des. Monoeres. Polym, 2005, 1, 25. The resulting polymer (i.e. poly-3,3-dimethylmalic acid) is water soluble as the free acid, has pendant carboxylic acid groups on each unit of the polymer chain and as well it has been reported that 3,3-dimethylmalic acid is a nontoxic molecule.

One could polymerize 4-benzyloxycarbonyl-,3,3-dimethyl-2-oxetanone in the presence of 3,5-dimethyl-1,4-dioxane-2,5-dione (DDD) and 3-butyrolactone to generate a block copolymer with pendant carboxylic acid groups as shown by Coulembier et al., Macromolecules, 2006, 39, 4001-4008. This polymerization reaction was carried out with a carbene catalyst in the presence of ethylene glycol. The catalyst used was a triazole carbene catalyst which leads to polymers with narrow polydispersities.

### Example 12

### Synthesis of PLGA-Histrelin Conjugate

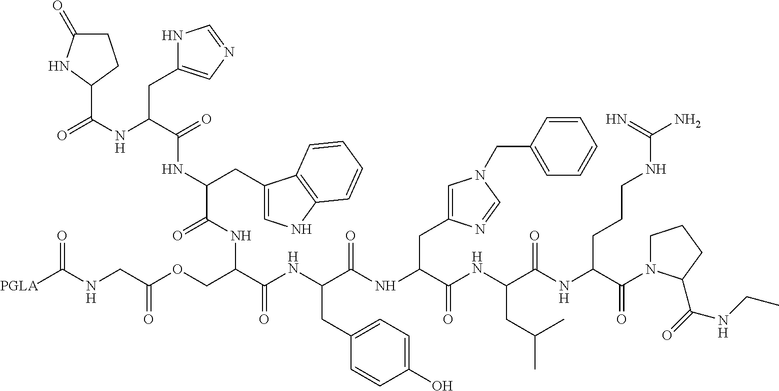

A PLGA5050, PLGA75/25 or PLGA85/15 polymer (recommended MW range from 10-100 kDa, but not exclusively limited to) will be conjugated to histrelin by using a glycine linker that is modified on the hydroxyl group on serine of histrelin. This ester linker between glycine and the therapeutic peptide can be cleaved off at high pH or by an enzyme such as estearase. ¹H NMR will be used to confirm consistency of the product. HPLC shall be used to analyze the purity of the product. GPC will be used to determine the purity, molecular weight and polydispersity of the product.

### Example 13

### Synthesis of PLGA-Nesiritide Conjugate

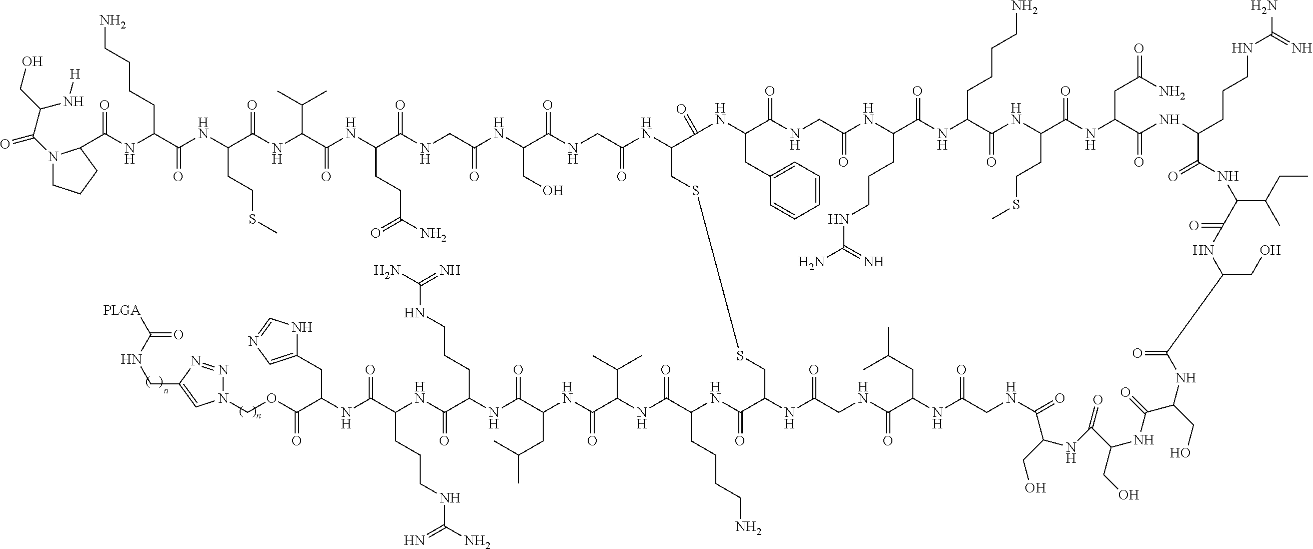

A PLGA5050, PLGA75/25 or PLGA85/15 polymer (recommended MW range from 10-100 kDa, but not exclusively limited to) will be modified at the carbonyl end group with an alkynyl functional group. Nesiritide will be functionalized with an azide group at the carbonyl end of histidine group. PLGA with an alkynyl group will then be conjugated to nesiritide with an azide group to form triazole by click chemistry. This ester linker between triazole and the therapeutic peptide can be cleaved off at high pH or by an enzyme such as estearase. ¹H NMR will be used to confirm consistency of the product. HPLC shall be used to analyze the purity of the product. GPC will be used to determine the purity, molecular weight and polydispersity of the product.

### Example 14

### Synthesis of PLGA-Thymopentin

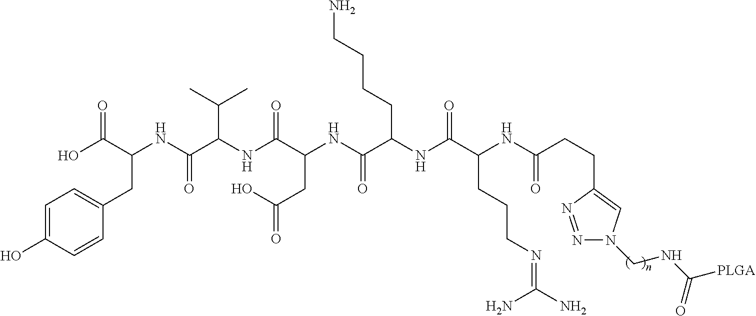

A PLGA5050, PLGA75/25 or PLGA85/15 polymer (recommended MW range from 10-100 kDa, but not exclusively limited to) will be modified at the carbonyl end group with an azide functional group. Thymopentin will be functionalized with an alkynyl group at the amino end of an arginine group. PLGA with an azide group will then be conjugated to thymopentin with an alknyl group to form triazole by click chemistry. ¹H NMR will be used to confirm consistency of the product. HPLC shall be used to analyze the purity of the product. GPC will be used to determine the purity, molecular weight and polydispersity of the product.

### Example 15

### Synthesis of PLGA-RWJ-800088

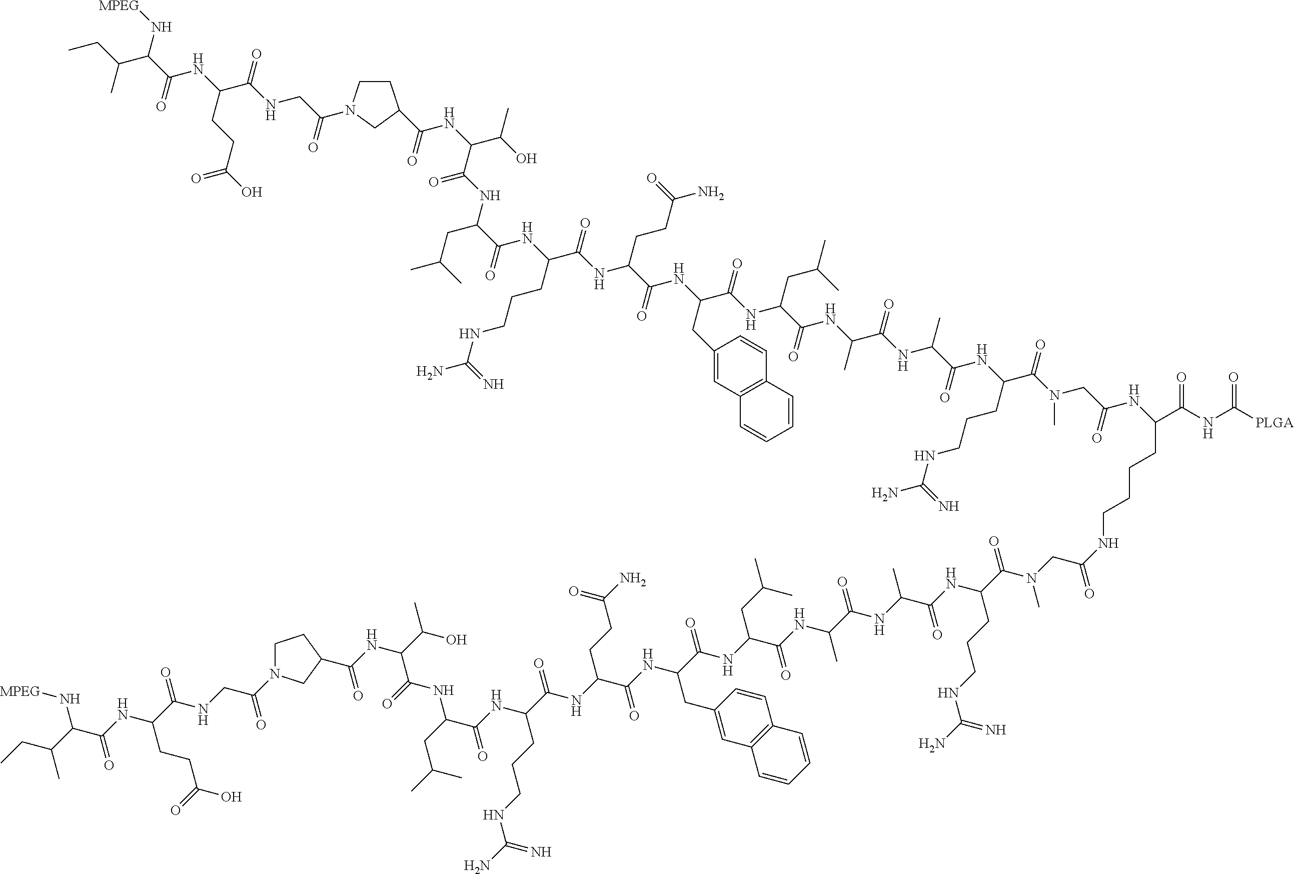

A PLGA5050, PLGA75/25 or PLGA85/15 polymer (recommended MW range from 10-100 kDa, but not exclusively limited to) will be conjugated to RWJ-800088 by formation of an amide bond between PLGA and the amino end group of lysine on RWJ-800088. ¹H NMR will be used to confirm consistency of the product. HPLC shall be used to analyze the purity of the product. GPC will be used to determine the purity, molecular weight and polydispersity of the product.

What is claimed is:

1. A particle comprising:
a) a plurality of hydrophobic polymers;
b) a plurality of hydrophilic-hydrophobic polymers; and
c) a plurality of therapeutic peptides or proteins, wherein at least a portion of the plurality of therapeutic peptides or proteins is covalently attached to either of a hydrophobic polymer of a) or a hydrophilic-hydrophobic polymer b).

2. The particle of claim 1, wherein at least a portion of the hydrophobic polymers of a) is not covalently attached to a therapeutic peptide or protein of c).

3. The particle of claim 1, wherein at least a portion of the hydrophobic polymers of a) is covalently attached to a therapeutic peptide or protein of c).

4. The particle of claim 3, wherein the at least a portion of the therapeutic peptides or proteins of c) is covalently attached to the hydrophobic polymer via a linker.

5. The particle of claim 3, wherein at least a portion of the hydrophobic polymers of a) is covalently attached to at least a portion of the therapeutic peptides or proteins of c) through an amino acid side chain of the therapeutic peptide or protein.

6. The particle of claim 1, wherein at least a portion of the hydrophilic-hydrophobic polymers of b) is covalently attached to a therapeutic peptide or protein of c).

7. The particle of claim 6, wherein at least a portion of the hydrophilic-hydrophobic polymers of b) is directly covalently attached to a therapeutic peptide or protein of c).

8. The particle of claim 6, wherein at least a portion of the therapeutic peptides or proteins of c) is covalently attached to a hydrophilic-hydrophobic polymer of b) via a linker.

9. The particle of claim 6, wherein at least a portion of the hydrophilic-hydrophobic polymers of b) is covalently attached to at least a portion of the therapeutic peptides or proteins of c) through an amino acid side chain of the therapeutic peptide or protein.

10. The particle of claim 1, wherein the particle further comprises a plurality of additional therapeutic peptides or proteins, wherein the additional therapeutic peptides or proteins differ from the therapeutic peptides or proteins of c).

11. The particle of claim 10, wherein at least a portion of the plurality of additional therapeutic peptides or proteins are attached to at least a portion of either the hydrophobic polymers of a) and/or the hydrophilic-hydrophobic polymers of b).

12. The particle of claim 1, further comprising a counterion.

13. A particle comprising:
a) optionally, a plurality of hydrophobic polymers;
b) a plurality of hydrophilic-hydrophobic polymer-conjugates, wherein the hydrophilic-hydrophobic polymer conjugate comprises a hydrophilic-hydrophobic polymer attached to a charged peptide or a charged protein; and
c) a plurality of charged therapeutic peptides or charged proteins, wherein the charge of the therapeutic peptide or protein is opposite the charge of the peptide or protein conjugated to the hydrophilic-hydrophobic polymer, and wherein the charged therapeutic peptide or protein forms a non-covalent bond (e.g., an ionic bond) with the charged peptide or the charged protein of the hydrophilic-hydrophobic polymer-conjugate.

14. The particle of claim 13, wherein the particle is substantially free of hydrophobic polymers.

15. The particle of claim 13, wherein the hydrophobic-hydrophilic polymer of the conjugate of b) is covalently attached to the charged peptide via a linker.

16. The particle of claim 1, wherein at least a portion of the hydrophobic polymers of a) are copolymers of lactic and glycolic acid (i.e., PLGA).

17. The particle of claim 16, wherein a portion of the hydrophobic polymers of a) are PLGA having a ratio of about 50:50 of lactic acid to glycolic acid.

18. The particle of claim 1, wherein the hydrophobic portion of the hydrophilic-hydrophobic polymers of b) comprises copolymers of lactic and glycolic acid (i.e., PLGA).

19. The particle of claim 18, wherein the hydrophobic portion of the hydrophilic-hydrophobic polymers of b) comprises PLGA having a ratio of about 50:50 of lactic acid to glycolic acid.

20. The particle of claim 1, wherein the hydrophilic portion of the hydrophilic-hydrophobic polymers of b) comprises PEG.

21. The particle of claim 1, wherein the therapeutic peptide comprises from about 2 to about 60 amino acid residues.

22. The particle of claim 1, wherein the therapeutic peptide or protein is selected from a therapeutic peptide or protein described herein.

23. The particle of claim 1, further comprising a surfactant.

24. The particle of claim 1, wherein the diameter of the particle is less than about 200 nm (e.g., less than about 150 nm).

25. The particle of claim 1, wherein the zeta potential of the particle is from about −20 to about +20 mV (e.g., from about −5 to about +5 mV).

26. A particle comprising:
a) a plurality of hydrophobic polymers;
b) a plurality of hydrophilic-hydrophobic polymers; and
c) a protein, wherein the protein is covalently attached to either a hydrophobic polymer of a) or a hydrophilic-hydrophobic polymer of b).

27. A composition comprising a plurality of particles of claim 1.

28. A composition comprising a plurality of particles of claim 26.

29. A kit comprising a plurality of particles of claim 1.

30. A single dosage unit comprising a plurality of particles of claim 1.

31. A method of treating a subject having a disorder comprising administering to said subject an effective amount of particles of claim 1.

32. A therapeutic peptide-hydrophobic polymer conjugate comprising a therapeutic peptide covalently attached to a hydrophobic polymer or a protein-hydrophobic polymer conjugate comprising a protein covalently attached to a hydrophobic polymer.

33. The therapeutic peptide-hydrophobic polymer conjugate or protein-hydrophobic polymer conjugate of claim 32, wherein the therapeutic peptide or protein is covalently attached to the hydrophobic polymer via the carboxy terminal of the therapeutic peptide or protein.

34. The therapeutic peptide-hydrophobic polymer conjugate or protein-hydrophobic polymer conjugate of claim 32, wherein the therapeutic peptide or protein is covalently attached to the hydrophobic polymer via the amino terminal of the therapeutic peptide or protein.

35. The therapeutic peptide-hydrophobic polymer conjugate or protein-hydrophobic polymer conjugate of claim 32, wherein the therapeutic peptide or protein is covalently attached to the hydrophobic polymer via an amino acid side chain of the therapeutic peptide or protein.

36. The therapeutic peptide-hydrophobic polymer conjugate or protein-hydrophobic polymer conjugate of claim 32, wherein the therapeutic peptide or protein is covalently attached to the hydrophobic polymer at a terminal end of the polymer.

37. The therapeutic peptide-hydrophobic polymer conjugate or protein-hydrophobic polymer conjugate of claim 32, wherein the therapeutic peptide or protein is covalently attached to the polymer along the backbone of the hydrophobic polymer.

38. The therapeutic peptide-hydrophobic polymer conjugate or protein-hydrophobic polymer conjugate of claim 32, wherein the therapeutic peptide or protein is covalently attached to the hydrophobic polymer via a linker.

39. A composition comprising a plurality of therapeutic peptide-hydrophobic polymer conjugates or protein-hydrophobic polymer conjugates of claim 32.

40. A method of making a therapeutic peptide-hydrophobic polymer conjugate or protein-hydrophobic polymer conjugate of claim 32, the method comprising:
providing a therapeutic peptide or protein and a polymer; and
subjecting the therapeutic peptide or protein and polymer to conditions that effect the covalent attachment of the therapeutic peptide or protein to the polymer.

41. A therapeutic peptide-hydrophilic-hydrophobic polymer conjugate or a protein-hydrophilic-hydrophobic polymer conjugate comprising a therapeutic peptide or protein covalently attached to a hydrophilic-hydrophobic polymer, wherein the hydrophilic-hydrophobic polymer comprises a hydrophilic portion attached to a hydrophobic portion.

42. The therapeutic peptide-hydrophilic-hydrophobic polymer conjugate or protein-hydrophilic-hydrophobic polymer conjugate of claim 41, wherein the therapeutic peptide or protein is attached to the hydrophilic portion of the hydrophilic-hydrophobic polymer.

43. The therapeutic peptide-hydrophilic-hydrophobic polymer conjugate or protein-hydrophilic-hydrophobic polymer conjugate of claim 41, wherein the therapeutic peptide or protein is attached to the hydrophobic portion of the hydrophilic-hydrophobic polymer.

44. The therapeutic peptide-hydrophilic-hydrophobic polymer conjugate or protein-hydrophilic-hydrophobic polymer conjugate of claim 41, wherein the hydrophilic-hydrophobic polymer is covalently attached to the therapeutic peptide or protein through the amino terminal of the therapeutic peptide or protein.

45. The therapeutic peptide-hydrophilic-hydrophobic polymer conjugate or protein-hydrophilic-hydrophobic polymer conjugate of claim 41, wherein the hydrophilic-hydrophobic polymer is covalently attached to the therapeutic peptide or protein through the carboxy terminal of the therapeutic peptide or protein.

46. The therapeutic peptide-hydrophilic-hydrophobic polymer conjugate or protein-hydrophilic-hydrophobic polymer conjugate of claim 41, wherein the hydrophilic-hydrophobic polymer is covalently attached to the therapeutic peptide or protein through an amino acid side chain of the therapeutic peptide or protein.

47. The therapeutic peptide-hydrophilic-hydrophobic polymer conjugate or protein-hydrophilic-hydrophobic polymer conjugate of claim 41, wherein the therapeutic peptide or protein is attached to the hydrophilic-hydrophobic polymer via a linker.

48. A composition comprising a plurality of therapeutic peptide-hydrophilic-hydrophobic polymer conjugates or protein-hydrophilic-hydrophobic polymer conjugates of claim 41.

49. A method of making a therapeutic peptide-hydrophilic-hydrophobic polymer conjugate or a protein-hydrophilic-hydrophobic polymer conjugate of claim 41, the method comprising:
providing a therapeutic peptide or protein and a hydrophilic-hydrophobic polymer; and
subjecting the therapeutic peptide or protein and hydrophilic-hydrophobic polymer to conditions that effect the covalent attachment of the therapeutic peptide or protein to the polymer.

50. A method of storing a conjugate of claim 1, the method comprising:
(a) providing said conjugate, particle or composition disposed in a container;
(b) storing said conjugate, particle or composition; and
(c) moving said container to a second location or removing all or an aliquot of said conjugate, particle or composition, from said container.

51. The method of claim 50, wherein the conjugate, particle or composition stored is a re-constituted formulation.
